SEQUENCE LISTING (152 sequences)

SEQ ID NO: 1 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 2 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 4 - protein, 427 aa, Homo sapiens
MEWPARLCGLWALLLCAGGGGGGGGAAPTETQPPVTNLSVSVENLCTVIWTWNPPEGASSNCSLWYFSHFGDKQDKKIAPETRRSIEVPLNERICLQVGSQCSTNESEKPSILVEKCISPPEGDPESAVTELQCIWHNLSYMKCSWLPGRNTSPDTNYTLYYWHRSLEKIHQCENIFREGQYFGCSFDLTKVKDSSFEQHSVQIMVKDNAGKIKPSFNIVPLTSRVKPDPPHIKNLSFHNDDLYVQWENPQNFISRCLFYEVEVNNSQTETHNVFYVQEAKCENPEFERNVENTSCFMVPGVLPDTLNTVRIRVKTNKLCYEDDKLWSNWSQEMSIGKKRNSTLYITMLLIVPVIVAGAIIVLLLYLKRLKIIIFPPIPDPGKIFKEMFGDQNDDTLHWKKYDIYEKQTKEETDSVVLIENLKKASQ

SEQ ID NO: 5 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - protein, 107 aa, Homo sapiens [sequence omitted]

SEQ ID NO: 7 - protein, 380 aa, Homo sapiens
MAFVCLAIGCLYTFLISTTFGCTSSSDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLRFWLPFGFILILVIFVTGLLLRKPNTYPKMIPEFFCDT

SEQ ID NO: 8 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 9 - protein, 1367 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 10 - protein, 628 aa, Homo sapiens
MEPPDAPAQARGAPRLLLLAVLLAAHPDAQAEVRLSVPPLVEVMRGKSVILDCTPTGTHDHYMLEWFLTDRSGARPRLASAEMQGSELQVTMHDTRGRSPPYQLDSQGRLVLAEAQVGDERDYVCVVRAGAAGTAEATARLNVFAKPEATEVSPNKGTLSVMEDSAQEIATCNSRNGNPAPKITWYRNGQRLEVPVEMNPEGYMTSRTVREASGLLSLTSTLYLRLRKDDRDASFHCAAHYSLPEGRHGRLDSPTFHLTLHYPTEHVQFWVGSPSTPAGWVREGDTVQLLCRGDGSPSPEYTLFRLQDEQEEVLNVNLEGNLTLEGVTRGQSGTYGCRVEDYDAADDVQLSKTLELRVAYLDPLELSEGKVLSLPLNSSAVVNCSVHGLPTPALRWTKDSTPLGDGPMLSLSSITFDSNGTYVCEASLPTVPVLSRTQNFTLLVQGSPELKTAEIEPKADGSWREGDEVTLICSARGHPDPKLSWSQLGGSPAEPIPGRQGWVSSSLTLKVTSALSRDGISCEASNPHGNKRHVFHFGTVSPQTSQAGVAVMAVAVSVGLLLLVVAVFYCVRRKGGPCCRQRREKGAPPPGEPGLSHSGSEQPEQTGLLMGGASGGARGGSGGFGDEC

SEQ ID NO: 11 - protein, 583 aa, Homo sapiens
MESKGASSCRLLFCLLISATVFRPGLGWYTVNSAYGDTIIIPCRLDVPQNLMFGKWKYEKPDGSPVFIAFRSSTKKSVQYDDVPEYKDRLNLSENYTLSISNARISDEKRFVCMLVTEDNVFEAPTIVKVFKQPSKPEIVSKALFLETEQLKKLGDCISEDSYPDGNITWYRNGKVLHPLEGAVVIIFKKEMDPVTQLYTMTSTLEYKTTKADIQMPFTCSVTYYGPSGQKTIHSEQAVFDIYYPTEQVTIQVLPPKNAIKEGDNITLKCLGNGNPPPEEFLFYLPGQPEGIRSSNTYTLTDVRRNATGDYKCSLIDKKSMIASTAITVHYLDLSLNPSGEVTRQIGDALPVSCTISASRNATVVWMKDNIRLRSSPSFSSLHYQDAGNYVCETALQEVEGLKKRESLTLIVEGKPQIKMTKKTDPSGLSKTIICHVEGFPKPAIQWTITGSGSVINQTEESPYINGRYYSKIIISPEENVTLTCTAENQLERTVNSLNVSAISIPEHDEADEISDENREKVNDQAKLIVGIVVGLLLAALVAGVVYWLYMKKSKTASKHVNKDLGNMEENKKLEENNHKTEA

SEQ ID NO: 12 - protein, 278 aa, Homo sapiens
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGYWFSVPLLLSIVSLVILLVLISILLYWKRHRNQDRGELSQGVQKMT

SEQ ID NO: 13 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 14 - protein, 427 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 15 - protein, 372 aa, Homo sapiens
MNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 16 - protein, 98 aa, Homo sapiens
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 17 - protein, 400 aa, Homo sapiens
MATLLLLLGVLVVSPDALGSTTAVQTPTSGEPLVSTSEPLSSKMYTTSITSDPKADSTGDQTSALPPSTSINEGSPLWTSIGASTGSPLPEPTTYQEVSIKMSSVPQETPHATSHPAVPITANSLGSHTVTGGTITTNSPETSSRTSGAPVTTAASSLETSRGTSGPPLTMATVSLETSKGTSGPPVTMATDSLETSTGTTGPPVTMTTGSLEPSSGASGPQVSSVKLSTMMSPTTSTNASTVPFRNPDENSRGMLPVAVLVALLAVIVLVALLLLWRRRQKRRTGALVLSRGGKRNGVVDAWAGPAQVPEEGAVTVTVGGSGGDKGSGFPDGEGSSRRPTLTTFFGRRKSRQGSLAMEELKSGSGPSLKGEEEPLVASEDGAVDAPAPDEPEGGDGAAP

SEQ ID NO: 18 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 19 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 20 - protein, 91 aa, Homo sapiens
MYGKIIFVLLLSEIVSISALSTTEVAMHTSTSSSVTKSYISSQTNGETGQLVHRFTVPAPVVIILIILCVMAGIIGTILLISYSIRRLIKA

SEQ ID NO: 21 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 22 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 23 - protein, 112 aa, Homo sapiens
MKGPPTFCSLLLLSLLLSPDPTAAFLLPPSTACCTQLYRKPLSDKLLRKVIQVELQEADGDCHLQAFVLHLAQRSICIHPQNPSLSQWFEHQERKLHGTLPKLNFGMLRKMG

SEQ ID NO: 24 - protein, 152 aa, Homo sapiens
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 25 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 26 - protein, 609 aa, Homo sapiens [sequence omitted]

SEQ ID NO: 27 - protein, 335 aa, Homo sapiens
MDPKGLLSLTFVLFLSLAFGASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKPWAVYAGLLGGVIMILIMVVILQLRRRGKTNHYQTTVEKKSLTIYAQVQKPGPLQKKLDSFPAQDPCTTIYVAATEPVPESVQETNSITVYASVTLPES

SEQ ID NO: 28 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 29 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 30 - protein, 904 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 31 - protein, 450 aa, Homo sapiens
MREIVHIQAGQCGNQIGAKFWEVISDEHGIDPSGNYVGDSDLQLERISVYYNEASSHKYVPRAILVDLEPGTMDSVRSGAFGHLFRPDNFIFGQSGAGNNWAKGHYTEGAELVDSVLDVVRKECENCDCLQGFQLTHSLGGGTGSGMGTLLISKVREEYPDRIMNTFSVVPSPKVSDTVVEPYNATLSIHQLVENTDETYCIDNEALYDICFRTLKLATPTYGDLNHLVSATMSGVTTSLRFPGQLNADLRKLAVNMVPFPRLHFFMPGFAPLTARGSQQYRALTVPELTQQMFDAKNMMAACDPRHGRYLTVATVFRGRMSMKEVDEQMLAIQSKNSSYFVEWIPNNVKVAVCDIPPRGLKMSSTFIGNSTAIQELFKRISEQFTAMFRRKAFLHWYTGEGMDEMEFTEAESNMNDLVSEYQQYQDATAEEEGEMYEDDEEESEAQGPK

SEQ ID NO: 32 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 33 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 34 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 35 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 36 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 37 - protein, 526 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 38 - protein, 252 aa, Homo sapiens
MGPPSASPHRECIPWQGLLLTASLLNFWNPPTTAKLTIESMPLSVAEGKEVLLLVHNLPQHLFGYSWYKGERVDGNSLIVGYVIGTQQATPGAAYSGRETIYTNASLLIQNVTQNDIGFYTLQVIKSDLVNEEATGQFHVYQENAPGLPVGAVAGIVTGVLVGVALVAALVCFLLLAKTGRTSIQRDLKEQQPQALAPGRGPSHSSAFSMSPLSTAQAPLPNPRTAASIYEELLKHDTNIYCRMDHKAEVAS

SEQ ID NO: 39 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 40 - protein, 344 aa, Homo sapiens
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDGPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI

SEQ ID NO: 41 - protein, 349 aa, Homo sapiens
MGPISAPSCRWRIPWQGLLLTASLFTFWNPPTTAQLTIEAVPSNAAEGKEVLLLVHNLPQDPRGYNWYKGETVDANRRIIGYVISNQQITPGPAYSNRETIYPNASLLMRNVTRNDTGSYTLQVIKLNLMSEEVTGQFSVHPETPKPSISSNNSNPVEDKDAVAFTCEPETQNTTYLWWVNGQSLPVSPRLQLSNGNRTLTLLSVTRNDVGPYECEIQNPASANFSDPVTLNVLYGPDAPTISPSDTYYHAGVNLNLSCHAASNPPSQYSWSVNGTFQQYTQKLFIPNITTKNSGSYACHTTNSATGRNRTTVRMITVSDALVQGSSPGLSARATVSIMIGVLARVALI

SEQ ID NO: 42 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 43 - protein, 362 aa, Homo sapiens
MLVMAPRTVLLLLSAALALTETWAGSHSMRYFYTSVSRPGRGEPRFISVGYVDDTQFVRFDSDAASPREEPRAPWIEQEGPEYWDRNTQIYKAQAQTDRESLRNLRGYYNQSEAGSHTLQSMYGCDVGPDGRLLRGHDQYAYDGKDYIALNEDLRSWTAADTAAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEPSSQSTVPIVGIVAGLAVLAVVVIGAVVAAVMCRRKSSGGKGGSYSQAACSDSAQGSDVSLTA

SEQ ID NO: 44 - protein, 236 aa, Homo sapiens
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 45 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 46 - protein, 99 aa, Homo sapiens
MKVSAALLCLLLMAATFSPQGLAQPDSVSIPITCCFNVINRKIPIQRLESYTRITNIQCPKEAVIFKTKRGKEVCADPKERWVRDSMKHLDQIFQNLKP

SEQ ID NO: 47 - protein, 164 aa, Homo sapiens
MASHSGPSTSVLFLFCCLGGWLASHTLPVRLLRPSDDVQKIVEELQSLSKMLLKDVEEEKGVLVSQNYTLPCLSPDAQPPNNIHSPAIRAYLKTIRQLDNKSVIDEIIEHLDKLIFQDAPETNISVPTDTHECKRFILTISQQFSECMDLALKSLTSGAQQATT

SEQ ID NO: 48 - protein, 430 aa, Homo sapiens
MSFTTRSTFSTNYRSLGSVQAPSYGARPVSSAASVYAGAGGSGSRISVSRSTSFRGGMGSGGLATGIAGGLAGMGGIQNEKETMQSLNDRLASYLDRVRSLETENRRLESKIREHLEKKGPQVRDWSHYFKIIEDLRAQIFANTVDNARIVLQIDNARLAADDFRVKYETELAMRQSVENDIHGLRKVIDDTNITRLQLETEIEALKEELLFMKKNHEEEVKGLQAQIASSGLTVEVDAPKSQDLAKIMADIRAQYDELARKNREELDKYWSQQIEESTTVVTTQSAEVGAAETTLTELRRTVQSLEIDLDSMRNLKASLENSLREVEARYALQMEQLNGILLHLESELAQTRAEGQRQAQEYEALLNIKVKLEAEIATYRRLLEDGEDFNLGDALDSSNSMQTIQKTTTRRIVDGKVVSETNDTKVLRH

SEQ ID NO: 49 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 50 - protein, 1255 aa, Homo sapiens [sequence omitted]

SEQ ID NO: 51 - protein, 766 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 52 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 53 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 54 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 55 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 56 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 57 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 58 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 59 - protein, 244 aa, Homo sapiens
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 60 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 61 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 62 - protein, 176 aa, Homo sapiens
MKASSLAFSLLSAAFYLLWTPSTGLKTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 63 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 64 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 65 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 66 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 67 - protein, 293 aa, Homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 68 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 69 - protein, 127 aa, Homo sapiens
MQQRGLAIVALAVCAALHASEAILPIASSCCTEVSHHISRRLLERVNMCRIQRADGDCDLAAVILHVKRRRICVSPHNHTVKQWMKVQAAKKNGKGNVCHRKKHHGKRNSNRAHQGKHETYGHKTPY

SEQ ID NO: 70 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 71 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 72 - protein, 205 aa, Homo sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 73 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 74 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 75 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 76 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 77 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 78 - protein, 285 aa, Homo sapiens
MVMRPLWSLLLWEALLPITVTGAQVLSKVGGSVLLVAARPPGFQVREAIWRSLWPSEELLATFFRGSLETLYHSRFLGRAQLHSNLSLELGPLESGDSGNFSVLMVDTRGQPWTQTLQLKVYDAVPRPVVQVFIAVERDAQPSKTCQVFLSCWAPNISEITYSWRRETTMDFGMEPHSLFTDGQVLSISLGPGDRDVAYSCIVSNPVSWDLATVTPWDSCHHEAAPGKASYKDVLLVVVPVSLLLMLVTLFSAWHWCPCSGKKKKDVHADRVGPETENPLVQDLP

SEQ ID NO: 79 - protein, 858 aa, Homo sapiens
MLQTKDLIWTLFFLGTAVSLQVDIVPSQGEISVGESKFFLCQVAGDAKDKDISWFSPNGEKLTPNQQRISVVWNDDSSSTLTIYNANIDDAGIYKCVVTGEDGSESEATVNVKIFQKLMFKNAPTPQEFREGEDAVIVCDVVSSLPPTIIWKHKGRDVILKKDVRFIVLSNNYLQIRGIKKTDEGTYRCEGRILARGEINFKDIQVIVNVPPTIQARQNIVNATANLGQSVTLVCDAEGFPEPTMSWTKDGEQIEQEEDDEKYIFSDDSSQLTIKKVDKNDEAEYICIAENKAGEQDATIHLKVFAKPKITYVENQTAMELEEQVTLTCEASGDPIPSITWRTSTRNISSEEKASWTRPEKQETLDGHMVVRSHARVSSLTLKSIQYTDAGEYICTASNTIGQDSQSMYLEVQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDQVEPYSSTAQVQFDEPEATGGVPILKYKAEWRAVGEEVWHSKWYDAKEASMEGIVTIVGLKPETTYAVRLAALNGKGLGEISAASEFKTQPVQGEPSAPKLEGQMGEDGNSIKVNLIKQDDGGSPIRHYLVRYRALSSEWKPEIRLPSGSDHVMLKSLDWNAEYEVYVVAENQQGKSKAAHFVFRTSAQPTAIPANGSPTSGLSTGAIVGILIVIFVLLLVVVDITCYFLNKCGLFMCIAVNLCGKAGPGAKGKDMEEGKAAFSKDESKEPIVEVRTEEERTPNHDGGKHTEPNETTPLTEPEKGPVEAKPECQETETKPAPAEVKTVPNDATQTKENESKA

SEQ ID NO: 80 - protein, 200 aa, Homo sapiens
MKLDMTGDCTPVLVLMAAVLTVTGAVPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 81 - protein, 503 aa, Homo sapiens
MLSQLAMLQGSLLLVVATMSVAQQTRQEADRGCETLVVQHGHCSYTFLLPKSEPCPPGPEVSRDSNTLQRESLANPLHLGKLPTQQVKQLEQALQNNTQWLKKLERAIKTILRSKLEQVQQQMAQNQTAPMLELGTSLLNQTTAQIRKLTDMEAQLLNQTSRMDAQMPETFLSTNKLENQLLLQRQKLQQLQGQNSALEKRLQALETKQQEELASILSKKAKLLNTLSRQSAALTNIERGLRGVRHNSSLLQDQQHSLRQLLVLLRHLVQERANASAPAFIMAGEQVFQDCAEIQRSGASASGVYTIQVSNATKPRKVFCDLQSSGGRWTLIQRRENGTVNFQRNWKDYKQGFGDPAGEHWLGNEVVHQLTRRAAYSLRVELQDWEGHEAYAQYEHFHLGSENQLYRLSVVGYSGSAGRQSSLVLQNTSFSTLDSDNDHCLCKCAQVMSGGWWFDACGLSNLNGVYYHAPDNKYKMDGIRWHYFKGPSYSLRASRMMIRPLDI

SEQ ID NO: 82 - protein, 99 aa, Homo sapiens
MKASAALLCLLLTAAAFSPQGLAQPVGINTSTTCCYRFINKKIPKQRLESYRRTTSSHCPREAVIFKTKLDKEICADPTQKWVQDFMKHLDKKTQTPKL

SEQ ID NO: 83 - protein, 260 aa, Homo sapiens
MRPEDRMFHIRAVILRALSLAFLLSLRGAGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 84 - protein, 967 aa, Homo sapiens
MAKGFYISKSLGILGILLGVAAVCTIIALSVVYSQEKNKNANSSPVASTTPSASATTNPASATTLDQSKAWNRYRLPNTLKPDSYRVTLRPYLTPNDRGLYVFKGSSTVRFTCKEATDVIIIHSKKLNYTLSQGHRVVLRGVGGSQPPDIDKTELVEPTEYLVVHLKGSLVKDSQYEMDSEFEGELADDLAGFYRSEYMEGNVRKVVATTQMQAADARKSFPCFDEPAMKAEFNITLIHPKDLTALSNMLPKGPSTPLPEDPNWNVTEFHTTPKMSTYLLAFIVSEFDYVEKQASNGVLIRIWARPSAIAAGHGDYALNVTGPILNFFAGHYDTPYPLPKSDQIGLPDFNAGAMENWGLVTYRENSLLFDPLSSSSSNKERVVTVIAHELAHQWFGNLVTIEWWNDLWLNEGFASYVEYLGADYAEPTWNLKDLMVLNDVYRVMAVDALASSHPLSTPASEINTPAQISELFDAISYSKGASVLRMLSSFLSEDVFKQGLASYLHTFAYQNTIYLNLWDHLQEAVNNRSIQLPTTVRDIMNRWTLQMGFPVITVDTSTGTLSQEHFLLDPDSNVTRPSEFNYVWIVPITSIRDGRQQQDYWLIDVRAQNDLFSTSGNEWVLLNLNVTGYYRVNYDEENWRKIQTQLQRDHSAIPVINRAQIINDAFNLASAHKVPVTLALNNTLFLIEERQYMPWEAALSSLSYFKLMFDRSEVYGPMKNYLKKQVTPLFIHFRNNTNNWREIPENLMDQYSEVNAISTACSNGVPECEEMVSGLFKQWMENPNNNPIHPNLRSTVYCNAIAQGGEEEWDFAWEQFRNATLVNEADKLRAALACSKELWILNRYLSYTLNPDLIRKQDATSTIISITNNVIGQGLVWDFVQSNWKKLFNDYGGGSFSFSNLIQAVTRRFSTEYELQQLEQFKKDNEETGFGSGTRALEQALEKTKANIKWVKENKEVVLQWFTENSK

SEQ ID NO: 85 - protein, 159 aa, Homo sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 86 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 87 - protein, 101 aa, Homo sapiens
MSSAAGFCASRPGLLFLGLLLLPLVVAFASAEAEEDGDLQCLCVKTTSQVRPRHITSLEVIKAGPHCPTAQLIATLKNGRKICLDLQAPLYKKIIKKLLES

SEQ ID NO: 88 - protein, 401 aa, Homo sapiens
MNNLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 89 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 90 - protein, 1039 aa, Homo sapiens
MARALCPLQALWLLEWVLLLLGPCAAPPAWALNLDPVQLTFYAGPNGSQFGFSLDFHKDSHGRVAIVVGAPRTLGPSQEETGGVFLCPWRAEGGQCPSLLFDLRDETRNVGSQTLQTFKARQGLGASVVSWSDVIVACAPWQHWNVLEKTEEAEKTPVGSCFLAQPESGRRAEYSPCRGNTLSRIYVENDFSWDKRYCEAGFSSVVTQAGELVLGAPGGYYFLGLLAQAPVADIFSSYRPGILLWHVSSQSLSFDSSNPEYFDGYWGYSVAVGEFDGDLNTTEYVVGAPTWSWTLGAVEILDSYYQRLHRLRGEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLVQDSLNPAVKSCVLPQTKTPVSCFNIQMCVGATGHNIPQKLSLNAELQLDRQKPRQGRRVLLLGSQQAGTTLNLDLGGKHSPICHTTMAFLRDEADFRDKLSPIVLSLNVSLPPTEAGMAPAVVLHGDTHVQEQTRIVLDCGEDDVCVPQLQLTASVTGSPLLVGADNVLELQMDAANEGEGAYEAELAVHLPQGAHYMRALSNVEGFERLICNQKKENETRVVLCELGNPMKKNAQIGIAMLVSVGNLEEAGESVSFQLQIRSKNSQNPNSKIVLLDVPVRAEAQVELRGNSFPASLVVAAEEGEREQNSLDSWGPKVEHTYELHNNGPGTVNGLHLSIHLPGQSQPSDLLYILDIQPQGGLQCFPQPPVNPLKVDWGLPIPSPSPIHPAHHKRDRRQIFLPEPEQPSRLQDPVLVSCDSAPCTVVQCDLQEMARGQRAMVTVLAFLWLPSLYQRPLDQFVLQSHAWFNVSSLPYAVPPLSLPRGEAQVWTQLLRALEERAIPIWWVLVGVLGGLLLLTILVLAMWKVGFFKRNRPPLEEDDEEGE

SEQ ID NO: 91 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 92 - protein, 993 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 93 - protein, 202 aa, Homo sapiens
MKVLAAGVVPLLLVLHWKHGAGSPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 94 - protein, 253 aa, Homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 95 - protein, 359 aa, Homo sapiens
MAEAITYADLRFVKAPLKKSISSRLGQDPGADDDGEITYENVQVPAVLGVPSSLASSVLGDKAAVKSEQPTASWRAVTSPAVGRILPCRTTCLRYLLLGLLLTCLLLGVTAICLGVRYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 96 - protein, 365 aa, Homo sapiens
MAVMAPRTLLLLLSGALALTQTWAGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 97 - protein, 366 aa, Homo sapiens
MRVMAPRALLLLLSGGLALTETWACSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKA

SEQ ID NO: 98 - protein, 1152 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 99 - protein, 252 aa, Homo sapiens
MGVLLTQRTLLSLVLALLFPSMASMAAIGSCSKEYRVLLGQLQKQTDLMQDTSRLLDPYIRIQGLDVPKLREHCRERPGAFPSEETLRGLGRRGFLQTLNATLGCVLHRLADLEQRLPKAQDLERSGLNIEDLEKLQMARPNILGLRNNIYCMAQLLDNSDTAEPTKAGRGASQPPTPTPASDAFQRKLEGCRFLHGYHRFMHSVGRVFSKWGESPNRSRRHSPHQALRKGVRRTRPSRKGKRLMTRGQLPR

SEQ ID NO: 100 - protein, 375 aa, Homo sapiens
MADHSFSDGVPSDSVEAAKNASNTEKLTDQVMQNPRVLAALQERLDNVPHTPSSYIETLPKAVKRRINALKQLQVRCAHIEAKFYEEVHDLERKYAALYQPLFDKRREFITGDVEPTDAESEWHSENEEEEKLAGDMKSKVVVTEKAAATAEEPDPKGIPEFWFTIFRNVDMLSELVQEYDEPILKHLQDIKVKFSDPGQPMSFVLEFHFEPNDYFTNSVLTKTYKMKSEPDKADPFSFEGPEIVDCDGCTIDWKKGKNVTVKTIKKKQKHKGRGTVRTITKQVPNESFFNFFNPLKASGDGESLDEDSEFTLASDFEIGHFFRERIVPRAVLYFTGEAIEDDDNFEEGEEGEEEELEGDEEGEDEDDAEINPKV

SEQ ID NO: 101 - protein, 835 aa, Homo sapiens
MGGRVFLAFCVWLTLPGAETQDSRGCARWCPQNSSCVNATACRCNPGFSSFSEIITTPTETCDDINECATPSKVSCGKFSDCWNTEGSYDCVCSPGYEPVSGAKTFKNESENTCQDVDECQQNPRLCKSYGTCVNTLGSYTCQCLPGFKFIPEDPKVCTDVNECTSGQNPCHSSTHCLNNVGSYQCRCRPGWQPIPGSPNGPNNTVCEDVDECSSGQHQCDSSTVCFNTVGSYSCRCRPGWKPRHGIPNNQKDTVCEDMTFSTWTPPPGVHSQTLSRFFDKVQDLGRDSKTSSAEVTIQNVIKLVDELMEAPGDVEALAPPVRHLIATQLLSNLEDIMRILAKSLPKGPFTYISPSNTELTLMIQERGDKNVTMGQSSARMKLNWAVAAGAEDPGPAVAGILSIQNMTTLLANASLNLHSKKQAELEEIYESSIRGVQLRRLSAVNSIFLSHNNTKELNSPILFAFSHLESSDGEAGRDPPAKDVMPGPRQELLCAFWKSDSDRGGHWATEGCQVLGSKNGSTTCQCSHLSSFAILMAHYDVEDWKLTLITRVGLALSLFCLLLCILTFLLVRPIQGSRTTIHLHLCICLFVGSTIFLAGIENEGGQVGLRCRLVAGLLHYCFLAAFCWMSLEGLELYFLVVRVFQGQGLSTRWLCLIGYGVPLLIVGVSAAIYSKGYGRPRYCWLDFEQGFLWSFLGPVTFIILCNAVIFVTTVWKLTQKFSEINPDMKKLKKARALTITAIAQLFLLGCTWVFGLFIFDDRSLVLTYVFTILNCLQGAFLYLLHCLLNKKVREEYRKWACLVAGGSKYSEFTSTTSGTGHNQTRALRASESGI

SEQ ID NO: 102 - protein, 658 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA

SEQ ID NO: 103 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 104 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 105 - protein, 478 aa, Homo sapiens
MGRCCFYTAGTLSLLLLVTSVTLLVARVFQKAVDQSIEKKIVLRNGTEAFDSWEKPPLPVYTQFYFFNVTNPEEILRGETPRVEEVGPYTYRELRNKANIQFGDNGTTISAVSNKAYVFERDQSVGDPKIDLIRTLNIPVLTVIEWSQVHFLREIIEAMLKAYQQKLFVTHTVDELLWGYKDEILSLIHVFRPDISPYFGLFYEKNGTNDGDYVFLTGEDSYLNFTKIVEWNGKTSLDWWITDKCNMINGTDGDSFHPLITKDEVLYVFPSDFCRSVYITFSDYESVQGLPAFRYKVPAEILANTSDNAGFCIPEGNCLGSGVLNVSICKNGAPIIMSFPHFYQADERFVSAIEGMHPNQEDHETFVDINPLTGIILKAAKRFQINIYVKKLDDFVETGDIRTMVFPVMYLNESVHIDKETASRLKSMINTTLIITNIPYIIMALGVFFGLVFTWLACKGQGSMDEGTADERAPLIRT

SEQ ID NO: 106 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 107 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 108 - protein, 261 aa, Homo sapiens
MAESHLQSSLITASQFFEIWLHFDADGSGYLEGKELQNLIQELQQARKKAGLELSPEMKTFVDQYGQRDDGKIGIVELAHVLPTEENFLLLFRCQQLKSCEEFMKTWRKYDTDHSGFIETEELKNFLKDLLEKANKTVDDTKLAEYTDLMLKLFDSNNDGKLELTEMARLLPVQENFLLKFQGIKMCGKEFNKAFELYDQDGNGYIDENELDALLKDLCEKNKQDLDINNITTYKKNIMALSDGGKLYRTDLALILCAGDN

SEQ ID NO: 109 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 110 - protein, 339 aa, Homo sapiens
MWQLWASLCCLLVLANARSRPSFHPLSDELVNYVNKRNTTWQAGHNFYNVDMSYLKRLCGTFLGGPKPPQRVMFTEDLKLPASFDAREQWPQCPTIKEIRDQGSCGSCWAFGAVEAISDRICIHTNAHVSVEVSAEDLLTCCGSMCGDGCNGGYPAEAWNFWTRKGLVSGGLYESHVGCRPYSIPPCEHHVNGSRPPCTGEGDTPKCSKICEPGYSPTYKQDKHYGYNSYSVSNSEKDIMAEIYKNGPVEGAFSVYSDFLLYKSGVYQHVTGEMMGGHAIRILGWGVENGTPYWLVANSWNTDWGDNGFFKILRGQDHCGIESEVVAGIPRTDQYWEKI

SEQ ID NO: 111 - protein, 503 aa, Homo sapiens
MTLGSPRKGLLMLLMALVTQGDPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLALILGPVLALLALVALGVLGLWHVRRRQEKQRGLHSELGESSLILKASEQGDSMLGDLLDSDCTTGSGSGLPFLVQRTVARQVALVECVGKGRYGEVWRGLWHGESVAVKIFSSRDEQSWFRETEIYNTVLLRHDNILGFIASDMTSRNSSTQLWLITHYHEHGSLYDFLQRQTLEPHLALRLAVSAACGLAHLHVEIFGTQGKPAIAHRDFKSRNVLVKSNLQCCIADLGLAVMHSQGSDYLDIGNNPRVGTKRYMAPEVLDEQIRTDCFESYKWTDIWAFGLVLWEIARRTIVNGIVEDYRPPFYDVVPNDPSFEDMKKVVCVDQQTPTIPNRLAADPVLSGLAQMMRECWYPNPSARLTALRIKKTLQKISNSPEKPKVIQ

SEQ ID NO: 112 - protein, 580 aa, Homo sapiens
MEVPQPEPAPGSALSPAGVCGGAQRPGHLPGLLLGSHGLLGSPVRAAASSPVTTLTQTMHDLAGLGSETPKSQVGTLLFRSRSRLTHLSLSRRASESSLSSESSESSDAGLCMDSPSPMDPHMAEQTFEQAIQAASRIIRNEQFAIRRFQSMPVRLLGHSPVLRNITNSQAPDGRRKSEAGSGAASSSGEDKENDGFVFKMPWKPTHPSSTHALAEWASRREAFAQRPSSAPDLMCLSPDRKMEVEELSPLALGRFSLTPAEGDTEEDDGFVDILESDLKDDDAVPPGMESLISAPLVKTLEKEEEKDLVMYSKCQRLFRSPSMPCSVIRPILKRLERPQDRDTPVQNKRRRSVTPPEEQQEAEEPKARVLRSKSLCHDEIENLLDSDHRELIGDYSKAFLLQTVDGKHQDLKYISPETMVALLTGKFSNIVDKFVIVDCRYPYEYEGGHIKTAVNLPLERDAESFLLKSPIAPCSLDKRVILIFHCEFSSERGPRMCRFIRERDRAVNDYPSLYYPEMYILKGGYKEFFPQHPNFCEPQDYRPMNHEAFKDELKTFRLKTRSWAGERSRRELCSRLQDQ

SEQ ID NO: 113 - protein, 317 aa, Homo sapiens
MRAAAAGGGVRTAALALLLGALHWAPARCEEYDYYGWQAEPLHGRSYSKPPQCLDIPADLPLCHTVGYKRMRLPNLLEHESLAEVKQQASSWLPLLAKRCHSDTQVFLCSLFAPVCLDRPIYPCRSLCEAVRAGCAPLMEAYGFPWPEMLHCHKFPLDNDLCIAVQFGHLPATAPPVTKICAQCEMEHSADGLMEQMCSSDFVVKMRIKEIKIENGDRKLIGAQKKKKLLKPGPLKRKDTKRLVLHMKNGAGCPCPQLDSLAGSFLVMGRKVDGQLLLMAVYRWDKKNKEMKFAVKFMFSYPCSLYYPFFYGAAEPH

SEQ ID NO: 114 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 115 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 116 - protein, 650 aa, Homo sapiens
MASNMDREMILADFQACTGIENIDEAITLLEQNNWDLVAAINGVIPQENGILQSEYGGETIPGPAFNPASHPASAPTSSSSSAFRPVMPSRQIVERQPRMLDFRVEYRDRNVDVVLEDTCTVGEIKQILENELQIPVSKMLLKGWKTGDVEDSTVLKSLHLPKNNSLYVLTPDLPPPSSSSHAGALQESLNQNFMLIITHREVQREYNLNFSGSSTIQEVKRNVYDLTSIPVRHQLWEGWPTSATDDSMCLAESGLSYPCHRLTVGRRSSPAQTREQSEEQITDVHMVSDSDGDDFEDATEFGVDDGEVFGMASSALRKSPMMPENAENEGDALLQFTAEFSSRYGDCHPVFFIGSLEAAFQEAFYVKARDRKLLAIYLHHDESVLTNVFCSQMLCAESIVSYLSQNFITWAWDLTKDSNRARFLTMCNRHFGSVVAQTIRTQKTDQFPLFLIIMGKRSSNEVLNVIQGNTTVDELMMRLMAAMEIFTAQQQEDIKDEDEREARENVKREQDEAYRLSLEADRAKREAHEREMAEQFRLEQIRKEQEEEREAIRLSLEQALPPEPKEENAEPVSKLRIRTPSGEFLERRFLASNKLQIVFDFVASKGFPWDEYKLLSTFPRRDVTQLDPNKSLLEVKLFPQETLFLEAKE

SEQ ID NO: 117 - protein, 796 aa, Homo sapiens
MAETKDVFGQEPHPVEDDLYKERTRKRRKSDRDQRFRAFPSMEQSALKEYEKLESRTRRVLSNTYQKLIQSVFLDDSIPNGVKYLINRLLALIEKPTVDPIYIALFGSTGAGKSSLINAIIQQAMFLPVSGESICTSCIVQVSSGCCVQYEAKIHLLSDQEWREELKNLTKLLHRTEELSREEADAWNRDEAVEEATWKLQMIYGNGAESKNYEELLRAKPKRKIPTSRVITLKAEEAEELSIKLDPYIRTQRRDWDGEAAEMRIWPLIKHVEVTLPKSDLIPEGVVLVDIPGTGDFNSKRDEMWKKTIDKCSVIWVISDIERVSGGQAHEDLLNESIKACQRGFCRDVALVVTKMDKLHLPEYLRERKAGNQAIQSQREAVLERNEMIKLQRTRILKEKLKRKLPADFKVLEASDLVYTVSAQEYWQQALLTEEETEIPKLREYIRKSLLDKKKRTVTKYVTEAFGLLLLTDSFNSTQNLPNEHLHMSVLRRFAEEKVELLEKAIAQCFACMEQPLQEGVRTARTSYRCILRACLVRSKGNQGFHQTLKAVCLKNGIYASRTLARIDLNEALTQPVYDQIDPVFGSIFRTGKPTGSALMPHIDAFKQSLQEKMTEIGIRSGWKYDSCKKNFLIQEISAILGGLEDHILRRKRRIYESLTASVQSDLKLCYEEAAQITGKKACERMKDAIRRGVDRQVAEGMFERAQERMQHQFQQLKTGIVEKVKGSITTMLALASSQGDGLYKELADVGSEYKEMEKLHRSLREVAENARLRKGMQEFLLRASPSKAGPPGTSL

SEQ ID NO: 118 - protein, 256 aa, Homo sapiens
MAHRPPSPALASVLLALLLSGAARAAEIVGGHEAQPHSRPYMASLQMRGNPGSHFCGGTLIHPSFVLTAAHCLRDIPQRLVNVVLGAHNVRTQEPTQQHFSVAQVFLNNYDAENKLNDVLLIQLSSPANLSASVATVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGRP

SEQ ID NO: 119 - protein, 890 aa, Homo sapiens
MALRRSMGRPGLPPLPLPPPPRLGLLLAALASLLLPESAAAGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDRAGQQGPPHSRTSWVPVVLGVLTALVTAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 120 - protein, 784 aa, Homo sapiens
MQRLMMLLATSGACLGLLAVAAVAAAGANPAQRDTHSLLPTHRRQKRDWIWNQMHIDEEKNTSLPHHVGKIKSSVSRKNAKYLLKGEYVGKVFRVDAETGDVFAIERLDRENISEYHLTAVIVDKDTGENLETPSSFTIKVHDVNDNWPVFTHRLFNASVPESSAVGTSVISVTAVDADDPTVGDHASVMYQILKGKEYFAIDNSGRIITITKSLDREKQARYEIVVEARDAQGLRGDSGTATVLVTLQDINDNFPFFTQTKYTFVVPEDTRVGTSVGSLFVEDPDEPQNRMTKYSILRGDYQDAFTIETNPAHNEGIIKPMKPLDYEYIQQYSFIVEATDPTIDLRYMSPPAGNRAQVIINITDVDEPPIFQQPFYHFQLKENQKKPLIGTVLAMDPDAARHSIGYSIRRTSDKGQFFRVTKKGDIYNEKELDREVYPWYNLTVEAKELDSTGTPTGKESIVQVHIEVLDENDNAPEFAKPYQPKVCENAVHGQLVLQISAIDKDITPRNVKFKFILNTENNFTLTDNHDNTANITVKYGQFDREHTKVHFLPVVISDNGMPSRTGTSTLTVAVCKCNEQGEFTFCEDMAAQVGVSIQAVVAILLCILTITVITLLIFLRRRLRKQARAHGKSVPEIHEQLVTYDEEGGGEMDTTSYDVSVLNSVRRGGAKPPRPALDARPSLYAQVQKPPRHAPGAHGGPGEMAAMIEVKKDEADHDGDGPPYDTLHIYGYEGSESIAESLSSLGTDSSDSDVDYDFLNDWGPRFKMLAELYGSDPREELLY

SEQ ID NO: 121 - protein, 92 aa, Homo sapiens
MTKLEEHLEGIVNIFHQYSVRKGHFDTLSKGELKQLLTKELANTIKNIKDKAVIDEIFQGLDANQDEQVDFQEFISLVAIALKAAHYHTHKE

SEQ ID NO: 122 - protein, 529 aa, Homo sapiens
MAPARTMARARLAPAGIPAVALWLLCTLGLQGTQAGPPPAPPGLPAGADCLNSFTAGVPGFVLDTNASVSNGATFLESPTVRRGWDCVRACCTTQNCNLALVELQPDRGEDAIAACFLINCLYEQNFVCKFAPREGFINYLTREVYRSYRQLRTQGFGGSGIPKAWAGIDLKVQPQEPLVLKDVENTDWRLLRGDTDVRVERKDPNQVELWGLKEGTYLFQLTVTSSDHPEDTANVTVTVLSTKQTEDYCLASNKVGRCRGSFPRWYYDPTEQICKSFVYGGCLGNKNNYLREEECILACRGVQGGPLRGSSGAQATFPQGPSMERRHPVCSGTCQPTQFRCSNGCCIDSFLECDDTPNCPDASDEAACEKYTSGFDELQRIHFPSDKGHCVDLPDTGLCKESIPRWYYNPFSEHCARFTYGGCYGNKNNFEEEQQCLESCRGISKKDVFGLRREIPIPSTGSVEMAVAVFLVICIVVVVAILGYCFFKNQRKDFHGHHHHPPPTPASSTVSTTEDTEHLVYNHTTRPL

SEQ ID NO: 123 - protein, 92 aa, Homo sapiens
MSELEKAMVALIDVFHQYSGREGDKHKLKKSELKELINNELSHFLEEIKEQEVVDKVMETLDNDGDGECDFQEFMAFVAMVTTACHEFFEHE

SEQ ID NO: 124 - protein, 882 aa, Homo sapiens
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 125 - protein, 323 aa, Homo sapiens
MAYVPAPGYQPTYNPTLPYYQPIPGGLNVGMSVYIQGVASEHMKRFFVNFVVGQDPGSDVAFHFNPRFDGWDKVVFNTLQGGKWGSEERKRSMPFKKGAAFELVFIVLAEHYKVVVNGNPFYEYGHRLPLQMVTHLQVDGDLQLQSINFIGGQPLRPQGPPMMPPYPGPGHCHQQLNSLPTMEGPPTFNPPVPYFGRLQGGLTARRTIIIKGYVPPTGKSFAINFKVGSSGDIALHINPRMGNGTVVRNSLLNGSWGSEEKKITHNPFGPGQFFDLSIRCGLDRFKVYANGQHLFDFAHRLSAFQRVDTLEIQGDVTLSYVQI

SEQ ID NO: 126 - protein, 263 aa, Homo sapiens
MITFLPLLLGLSLGCTGAGGFVAHVESTCLLDDAGTPKDFTYCISFNKDLLTCWDPEENKMAPCEFGVLNSLANVLSQHLNQKDTLMQRLRNGLQNCATHTQPFWGSLTNRTRPPSVQVAKTTPFNTREPVMLACYVWGFYPAEVTITWRKNGKLVMPHSSAHKTAQPNGDWTYQTLSHLALTPSYGDTYTCVVEHIGAPEPILRDWTPGLSPMQTLKVSVSAVTLGLGLIIFSLGVISWRRAGHSSYTPLPGSNYSEGWHIS

SEQ ID NO: 127 - protein, 163 aa, Homo sapiens
MRRLLIPLALWLGAVGVGVAELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQFAFSKALPRS

SEQ ID NO: 128 - protein, 602 aa, Homo sapiens
MPYKLKKEKEPPKVAKCTAKPSSSGKDGGGENTEEAQPQPQPQPQPQAQSQPPSSNKRPSNSTPPPTQLSKIKYSGGPQIVKKERRQSSSRFNLSKNRELQKLPALKDSPTQEREELFIQKLRQCCVLFDFVSDPLSDLKFKEVKRAGLNEMVEYITHSRDVVTEAIYPEAVTMFSVNLFRTLPPSSNPTGAEFDPEEDEPTLEAAWPHLQLVYEFFLRFLESPDFQPNIAKKYIDQKFVLALLDLFDSEDPRERDFLKTILHRIYGKFLGLRAYIRRQINHIFYRFIYETEHHNGIAELLEILGSIINGFALPLKEEHKMFLIRVLLPLHKVKSLSVYHPQLAYCVVQFLEKESSLTEPVIVGLLKFWPKTHSPKEVMFLNELEEILDVIEPSEFSKVMEPLFRQLAKCVSSPHFQVAERALYYWNNEYIMSLISDNAARVLPIMFPALYRNSKSHWNKTIHGLIYNALKLFMEMNQKLFDDCTQQYKAEKQKGRFRMKEREEMWQKIEELARLNPQYPMFRAPPPLPPVYSMETETPTAEDIQLLKRTVETEAVQMLKDIKKEKVLLRRKSELPQDVYTIKALEAHKRAEEFLTASQEAL

SEQ ID NO: 129 - protein, 263 aa, Homo sapiens
MMPKHCFLGFLISFFLTGVAGTQSTHESLKPQRVQFQSRNFHNILQWQPGRALTGNSSVYFVQYKIMFSCSMKSSHQKPSGCWQHISCNFPGCRTLAKYGQRQWKNKEDCWGTQELSCDLTSETSDIQEPYYGRVRAASAGSYSEWSMTPRFTPWWETKIDPPVMNITQVNGSLLVILHAPNLPYRYQKEKNVSIEDYYELLYRVFIINNSLEKEQKVYEGAHRAVEIEALTPHSSYCVVAEIYQPMLDRRSQRSEERCVEIP

SEQ ID NO: 130 - protein, 208 aa, Homo sapiens
MAPLGEVGNYFGVQDAVPFGNVPVLPVDSPVLLSDHLGQSEAGGLPRGPAVTDLDHLKGILRRRQLYCRTGFHLEIFPNGTIQGTRKDHSRFGILEFISIAVGLVSIRGVDSGLYLGMNEKGELYGSEKLTQECVFREQFEENWYNTYSSNLYKHVDTGRRYYVALNKDGTPREGTRTKRHQKFTHFLPRPVDPDKVPELYKDILSQS

SEQ ID NO: 131 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 132 - protein, 79 aa, Homo sapiens
MKVTGIFLLSALALLSLSGNTGADSLGREAKCYNELNGCTKIYDPVCGTDGNTYPNECVLCFENRKRQTSILIQKSGPC

SEQ ID NO: 133 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 134 - protein, 303 aa, Homo sapiens
MRAWIFFLLCLAGRALAAPQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 135 - protein, 388 aa, Homo sapiens
MKWMVVVLVCLQLLEAAVVKVPLKKFKSIRETMKEKGLLGEFLRTHKYDPAWKYRFGDLSVTYEPMAYMDAAYFGEISIGTPPQNFLVLFDTGSSNLWVPSVYCQSQACTSHSRFNPSESSTYSTNGQTFSLQYGSGSLTGFFGYDTLTVQSIQVPNQEFGLSENEPGTNFVYAQFDGIMGLAYPALSVDEATTAMQGMVQEGALTSPVFSVYLSNQQGSSGGAVVFGGVDSSLYTGQIYWAPVTQELYWQIGIEEFLIGGQASGWCSEGCQAIVDTGTSLLTVPQQYMSALLQATGAQEDEYGQFLVNCNSIQNLPSLTFIINGVEFPLPPSSYILSNNGYCTVGVEPTYLSSQNGQPLWILGDVFLRSYYSVYDLGNNRVGFATAA

SEQ ID NO: 136 - protein, 460 aa, Homo sapiens
MFTIKLLLFIVPLVISSRIDQDNSSFDSLSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPETPEHPEVTSLKTFVEKQDNSIKDLLQTVEDQYKQLNQQHSQIKEIENQLRRTSIQEPTEISLSSKPRAPRTTPFLQLNEIRNVKHDGIPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPENKDLVFSTWDHKAKGHFNCPEGYSGGWWWHDECGENNLNGKYNKPRAKSKPERRRGLSWKSQNGRLYSIKSTKMLIHPTDSESFE

SEQ ID NO: 137 - protein, 259 aa, Homo sapiens
MSEVPVARVWLVLLLLTVQVGVTAGAPWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSKALHVTNIKKWKEPCRIELYRVVESLAKAQETSGEEISKFYLPNCNKNGFYHSRQCETSMDGEAGLCWCVYPWNGKRIPGSPEIRGDPNCQIYFNVQN

SEQ ID NO: 138 - protein, 171 aa, Homo sapiens
MLVNFILRCGLLLVTLSLAIAKHKQSSFTKSCYPRGTLSQAVDALYIKAAWLKATIPEDRIKNIRLLKKKTKKQFMKNCQFQEQLLSFFMEDVFGQLQLQGCKKIRFVEDFHSLRQKLSHCISCASSAREMKSITRMKRIFYRIGNKGIYKAISELDILLSWIKKLLESSQ

SEQ ID NO: 139 - protein, 385 aa, Homo sapiens
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 140 - protein, 1042 aa, Homo sapiens
MKQSPALAPEERCRRAGSPKPVLRADDNNMGNGCSQKLATANLLRFLLLVLIPCICALVLLLVILLSYVGTLQKVYFKSNGSEPLVTDGEIQGSDVILTNTIYNQSTVVSTAHPDQHVPAWTTDASLPGDQSHRNTSACMNITHSQCQMLPYHATLTPLLSVVRNMEMEKFLKFFTYLHRLSCYQHIMLFGCTLAFPECIIDGDDSHGLLPCRSFCEAAKEGCESVLGMVNYSWPDFLRCSQFRNQTESSNVSRICFSPQQENGKQLLCGRGENFLCASGICIPGKLQCNGYNDCDDWSDEAHCNCSENLFHCHTGKCLNYSLVCDGYDDCGDLSDEQNCDCNPTTEHRCGDGRCIAMEWVCDGDHDCVDKSDEVNCSCHSQGLVECRNGQCIPSTFQCDGDEDCKDGSDEENCSVIQTSCQEGDQRCLYNPCLDSCGGSSLCDPNNSLNNCSQCEPITLELCMNLPYNSTSYPNYFGHRTQKEASISWESSLFPALVQTNCYKYLMFFSCTILVPKCDVNTGEHIPPCRALCEHSKERCESVLGIVGLQWPEDTDCSQFPEENSDNQTCLMPDEYVEECSPSHFKCRSGQCVLASRRCDGQADCDDDSDEENCGCKERDLWECPSNKQCLKHTVICDGFPDCPDYMDEKNCSFCQDDELECANHACVSRDLWCDGEADCSDSSDEWDCVTLSINVNSSSFLMVHRAATEHHVCADGWQEILSQLACKQMGLGEPSVTKLIQEQEKEPRWLTLHSNWESLNGTTLHELLVNGQSCESRSKISLLCTKQDCGRRPAARMNKRILGGRTSRPGRWPWQCSLQSEPSGHICGCVLIAKKWVLTVAHCFEGRENAAVWKVVLGINNLDHPSVFMQTRFVKTIILHPRYSRAVVDYDISIVELSEDISETGYVRPVCLPNPEQWLEPDTYCYITGWGHMGNKMPFKLQEGEVRIISLEHCQSYFDMKTITTRMICAGYESGTVDSCMGDSGGPLVCEKPGGRWTLFGLTSWGSVCFSKVLGPGVYSNVSYFVEWIKRQIYIQTFLLN

SEQ ID NO: 141 - protein, 122 aa, Homo sapiens
MEASALTSSAVTSVAKVVRVASGSAVVLPLARIATVVIGGVVAMAAVPMVLSAMGFTAAGIASSSIAAKMMSAAAIANGGGVASGSLVATLQSLGATGLSGLTKFILGSIGSAIAAVIARFY

SEQ ID NO: 142 - protein, 132 aa, Homo sapiens
MAFDSTWKVDRSENYDKFMEKMGVNIVKRKLAAHDNLKLTITQEGNKFTVKESSAFRNIEVVFELGVTFNYNLADGTELRGTWSLEGNKLIGKFKRTDNGNELNTVREIIGDELVQTYVYEGVEAKRIFKKD

SEQ ID NO: 143 - protein, 325 aa, Homo sapiens
MLPRVGCPALPLPPPPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 144 - protein, 352 aa, Homo sapiens
MRSLGALLLLLSACLAVSAGPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPRVRRAVLPQEEEGSGGGQLVTEVTKKEDSCQLGYSAGPCMGMTSRYFYNGTSMACETFQYGGCMGNGNNFVTEKECLQTCRTVAACNLPIVRGPCRAFIQLWAFDAVKGKCVLFPYGGCQGNGNKFYSEKECREYCGVPGDGDEELLRFSN

SEQ ID NO: 145 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 146 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 147 - protein, 98 aa, Homo sapiens
MAAEPLTELEESIETVVTTFFTFARQEGRKDSLSVNEFKELVTQQLPHLLKDVGSLDEKMKSLDVNQDSELKFNEYWRLIGELAKEIRKKKDLKIRKK

SEQ ID NO: 148 - protein, 765 aa, Homo sapiens
MSGDHLHNDSQIEADFRLNDSHKHKDKHKDREHRHKEHKKEKDREKSKHSNSEHKDSEKKHKEKEKTKHKDGSSEKHKDKHKDRDKEKRKEEKVRASGDAKIKKEKENGFSSPPQIKDEPEDDGYFVPPKEDIKPLKRPRDEDDADYKPKKIKTEDTKKEKKRKLEEEEDGKLKKPKNKDKDKKVPEPDNKKKKPKKEEEQKWKWWEEERYPEGIKWKFLEHKGPVFAPPYEPLPENVKFYYDGKVMKLSPKAEEVATFFAKMLDHEYTTKEIFRKNFFKDWRKEMTNEEKNIITNLSKCDFTQMSQYFKAQTEARKQMSKEEKLKIKEENEKLLKEYGFCIMDNHKERIANFKIEPPGLFRGRGNHPKMGMLKRRIMPEDIIINCSKDAKVPSPPPGHKWKEVRHDNKVTWLVSWTENIQGSIKYIMLNPSSRIKGEKDWQKYETARRLKKCVDKIRNQYREDWKSKEMKVRQRAVALYFIDKLALRAGNEKEEGETADTVGCCSLRVEHINLHPELDGQEYVVEFDFLGKDSIRYYNKVPVEKRVFKNLQLFMENKQPEDDLFDRLNTGILNKHLQDLMEGLTAKVFRTYNASITLQQQLKELTAPDENIPAKILSYNRANRAVAILCNHQRAPPKTFEKSMMNLQTKIDAKKEQLADARRDLKSAKADAKVMKDAKTKKVVESKKKAVQRLEEQLMKLEVQATDREENKQIALGTSKLNYLDPRITVAWCKKWGVPIEKIYNKTQREKFAWAIDMADEDYEF

SEQ ID NO: 149 - protein, 2477 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 150 - protein, 91 aa, Homo sapiens
MKLAVTLTLVTLALCCSSASAEICPSFQRVIETLLMDTPSSYEAAMELFSPDQDMREAGAQLKKLVDTLPQKPRESIIKLMEKIAQSSLCN

SEQ ID NO: 151 - protein, 375 aa, Homo sapiens
MERASCLLLLLLPLVHVSATTPEPCELDDEDFRCVCNFSEPQPDWSEAFQCVSAVEVEIHAGGLNLEPFLKRVDADADPRQYADTVKALRVRRLTVGAAQVPAQLLVGALRVLAYSRLKELTLEDLKITGTMPPLPLEATGLALSSLRLRNVSWATGRSWLAELQQWLKPGLKVLSIAQAHSPAFSCEQVRAFPALTSLDLSDNPGLGERGLMAALCPHKFPAIQNLALRNTGMETPTGVCAALAAAGVQPHSLDLSHNSLRATVNPSAPRCMWSSALNSLNLSFAGLEQVPKGLPAKLRVLDLSCNRLNRAPQPDELPEVDNLTLDGNPFLVPGTALPHEGSMNSGVVPACARSTLSVGVSGTLVLLQGARGFA

SEQ ID NO: 152 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKD

(19) United States
(12) Patent Application Publication
(10) Pub. No.: US 2024/0219402 A1
(43) Pub. Date: Jul. 4, 2024

(54) METHODS FOR THE DETERMINATION OF THE PREDISPOSITION FOR A SEVERE OR CRITICAL COURSE OF A COVID-19-DISEASE FROM A MILD OR MODERATE COURSE OF A COVID-19-DISEASE IN A SUBJECT

(71) Applicant: SCIOMICS GMBH, Neckargemünd (DE)

(72) Inventors: Katrin Hufnagel, Heidelberg (DE); Nadine Stroh, Eppelheim (DE); Anne Griesbeck, Dossenheim (DE); Florian Skwirblies, Neckarsteinach (DE); Marco Klein, Neckargemünd (DE); Christoph Schröder, Heidelberg (DE)

(21) Appl. No.: 18/040,420

(22) PCT Filed: Jul. 22, 2021
(86) PCT No.: PCT/EP2021/070606

§ 371 Date: Feb. 2, 2023

(30) Foreign Application Priority Data
Aug. 3, 2020 (EP) 20189257.7

(51) Int. Cl. G01N 33/68

(52) U.S. Cl. CPC G01N 33/6893; G01N 2800/26; G01N 2800/52; G01N 2800/56

(57) ABSTRACT

A method to determine the predisposition for a severe or critical course of a COVID-19-disease from a mild or moderate course of a COVID-19-disease in a subject. The method can be used to stratify a patient-group, diagnose a SASR-CoV-2-infection, predict the course of a COVID-19-disease in a subject, supervise the therapy of a subject with COVID-19 and/or monitor the efficacy of existing and novel therapeutic agents against COVID-19.

### SUMMARY

A method is disclosed to determine the predisposition for a severe or critical course of a COVID-19-disease from a mild or moderate course of a COVID-19-disease in a subject. The method can be used to stratify a patient-group, diagnose a SASR-CoV-2-infection, predict the course of a COVID-19-disease in a subject, supervise the therapy of a subject with COVID-19 and/or monitor the efficacy of existing and novel therapeutic agents against COVID-19.

### BACKGROUND OF THE INVENTION

Coronavirus Disease 2019 (COVID-19) is caused by severe acute respiratory syndrome coronavirus 2 (SARS-CoV-2), a highly infectious, zoonotic virus that is believed to exploit angiotens-inconverting enzyme 2 (ACE2) as a cell entry receptor. Clinical presentation of COVID-19 involves a broad range of symptoms and disease trajectories.

Understanding the nature of the immune response that leads to recovery over severe disease is key to developing effective treatment against COVID-19. Coronaviruses, including Severe Acute Respiratory Syndrome (SARS-CoV) and Middle Eastern Respiratory Syndrome (MERS), typically induce strong inflammatory responses and associated lymphopenia.

Studies of COVID-19 patients have reported increases in inflammatory monocytes and neutrophils and a sharp decrease in lymphocytes, and an inflammatory milieu containing IL-1, IL-6, and TNF-α in severe disease. Despite these analyses, immune response dynamics during the course of SARS-CoV-2 infection and its possible correlation with clinical trajectory remain unknown.

A recent study was pre-published by Lucas et al. (Nature, “longitudinal analyses reveal immunological misfiring in severe COVID-19”, https://doi.org./10.1038/s41586-020-2588-y). The authors tested a number of cytokines and chemokines of COVID-19 patients and controls. In a statistical assessment a clear differentiation between a severe or moderate course of the disease was reported for individual biomarkers. However, early, medium as well as late phase samples were pooled for this assessment. In addition, the authors did not adjust p-values for multiple testing.

Therefore, no biomarkers are conclusively reported in this article predicting a severe or critical disease from samples at an early onset of the disease in a statistically significant manner.

Thus, the need still exists to identify biomarker-based methods in order to determinate the predisposition for a severe or critical course of a COVID-19-disease from a mild or moderate course of a COVID-19-disease in a subject.

### DETAILED DESCRIPTION OF THE INVENTION

The present invention analysed more than 349 biomarkers (proteins and non-proteins) in 53 blood samples from 16 COVID-19 patients at three different time-phases (discovery) and identified a number of biomarkers which allow the determination of the predisposition for a severe or critical course of a COVID-19-disease in a subject. In addition, 106 blood samples from 106 COVID-19 patients were analysed to validate the findings of acute phase.

Thus, in one aspect the present invention pertains to a method for determining the predisposition for a severe or critical course of a COVID-19-disease from a mild or moderate course of a COVID-19-disease in a subject, comprising the steps of:
a) determining in a sample obtained from the subject the amount of at least one biomarker;
b) determining the difference of the amount of said at least one biomarker to a reference amount for said at least one biomarker;
   wherein the reference amount is the amount of the respective biomarker in a subject who has a mild or moderate COVID-19-disease; and
   wherein the difference |log FC| is at least 0.5.

The difference of |log FC| of at least 0.5 compared to the reference amount indicates that the subject has a predisposition for a severe or critical course of a COVID-19-disease. In other embodiments a |log FC| of at least 1.0 is preferred, even more preferred a |log FC| of at least 1.5, even more preferred a |log FC| of at least 2.0, and a |log FC| up to 2.5, up to 3.0, or even a |log FC| up to 3.5, up to 4.0, up to 5.0. Thus, biomarkers of a log FC between 0.5 and 5.0 are preferred biomarkers, even more preferred a log FC between 1.0 and 4.0, yet even more preferred a log FC between 1.5 and 3.5, most preferred a log FC between 2.0 and 3.5.

The difference must also be significant in at least one time phase (i.e. acute, medium and/or late). The significance is depicted by the “adjusted p-value”, i.e. p-values adjusted for multiple testing and indicate the level of significance. “Significant” in this respect refers to an adjusted p-value of less than 0.05 (adjusted for multiple testing according to Bonferroni and Hochberg). In one embodiment the p-value is less than 0.05, in another embodiment the p-value is less than 0.01, in another embodiment the p-value is less than 0.005, in another embodiment the p-value is less than 0.001, in yet another embodiment the p-value is less than less than 5*10⁻³, in yet another embodiment the p-value is less than 1*10⁻³, in yet another embodiment the p-value is less than 5*10⁻⁴, in yet another embodiment the p-value is less than 1*10⁻⁴, in yet another embodiment the p-value is less than 5*10⁻⁵, in yet another embodiment the p-value is less than 1*10⁻⁵, in yet another embodiment the p-value is less than 5*10⁻⁶, in yet another embodiment the p-value is less than 1*10⁻⁶, and in yet another embodiment the p-value is up to 5*10⁻¹, in yet another embodiment the p-value is up to 1*10⁻¹, in yet another embodiment the p-value is up to 1*10⁻⁸, in yet another embodiment the p-value is up to 1*10⁻⁹, in yet another embodiment the p-value is up to 1*10⁻¹⁰.

Preferred “adjusted p-values” are in one embodiment between 4.3*10⁻¹⁰ and 5.5*10⁻³, in another embodiment between 1.2*10⁻⁹ and 3.4*10⁻², in another embodiment between 1.3*10⁻⁵ and 4.8*10⁻¹.

The term “significant in at least one time phase” means that the biomarker measured in one of the three time phases “acute”, “medium” and/or “late” is significantly up- or downregulated (the amount is significant in- or downregulated) with a adjusted p-value of less than 0.05. In one embodiment the biomarker is significant only during one time phase, in other embodiments during two, or all three time phases.

The subset of biomarkers may be selected according to the specific time phase of interest: For example, for differentiating methods during acute phase, biomarkers significantly up- or downregulated at least during acute phase are preferred. For differentiating methods during any specific phase of the COVID-19-disease, biomarkers being significantly up- or downregulated during that phase are preferred. For monitoring differentiating biomarkers during the whole course of a COVID-19-disease, biomarkers are preferred, which are significantly up- or downregulated during the whole course of the disease.

A biomarker suitable for the present invention does show both a significant adjusted p-value of at less than 0.5 as well as a reasonable high |log FC| of at least 0.5. In one embodiment the adjusted p-value is equal to or less than 3.3*10⁻¹ and a |log FC| of equal to or more than 0.61. In another embodiment the adjusted p-value is equal to or less than 1.2*10⁻⁵ and a |log FC| of equal to or more than 1.54. In another embodiment the adjusted p-value is equal to or less than 8.9*10⁵ and a |log FC| of equal to or more than 1.64.

The inventive method may be applied to a diagnostic method, a predictive method, a therapeutic method and or an analytic method, as described herein.

The term “predisposition” as used herein, refers to a susceptibility that a severe or critical course of COVID-19-disease takes place in a subject. This susceptibility may have genetic origins, and seems to be triggered by a SARS-CoV-2-infection particular in combination with environmental or lifestyle factors, such as age, tobacco smoking, diabetes mellitus, overweight or other preconditions. The biomarkers identified in this invention enable the skilled person to identify subjects who are predisposed to COVID-19, especially with severe or critical course of that disease.

This “predisposition” may also include the development of “long-covid” after the COVID-19-disease has been overcome. Long COVID, also known as post-COVID-19 syndrome, post-acute sequelae of COVID-19 (PASC), chronic COVID syndrome (CCS) and long-haul COVID, is a condition characterized by long-term sequelae-appearing or persisting after the typical convalescence period—of coronavirus disease 2019 (COVID-19). Long COVID can affect nearly every organ system with sequelae including respiratory system disorders, nervous system and neurocognitive disorders, mental health disorders, metabolic disorders, cardiovascular disorders, gastrointestinal disorders, malaise, fatigue, musculoskeletal pain, and anemia. A wide range of symptoms are commonly discussed, including fatigue, headaches, shortness of breath, anosmia (loss of smell), parosmia (distorted smell), muscle weakness, low fever and cognitive dysfunction.

The terms “mild”, “moderate”, “severe” and “critical” as used herein, were defined according to the WHO-China joint mission statement (Report of the WHO-China Joint Mission on Coronavirus Disease 2019 (COVID-19), 16-24 Feb. 2020).

A “mild course” of COVID-19 as used herein, is defined by patients positive for a SARS-CoV-2-infection without any signs of pneumonia.

A “moderate course” of COVID-19 as used herein, is defined by patients which show signs of pneumonia in imaging methods, but have a blood oxygen saturation of at least 93% and do not need oxygen therapy (ventilation).

A “severe course” of COVID-19 as used herein, is defined by patients which show symptoms of dyspnoea, a respiratory frequency of >30/minute, blood oxygen saturation of ≤93%, a PaO₂/FiO₂ ratio of <300, and/or lung infiltrates >50% of the lung field within 24-48 hours.

A “critical course” of COVID-19 as used herein, is defined by patients which are in need of noninvasive or invasive ventilation and/or further intensive care measures (dialysis, catecholamine therapy). Further symptoms are respiratory failure, septic shock, and/or multiple organ dysfunction/failure.

The term “mild-or-moderate” and the abbreviation “MM” and “MM-patient” as used herein, refer to patients which show either a mild course or a moderate course of COVID-19 as described above. Patients from this group served as the reference group for the biomarker assessment.

The term “critical-or-severe course” and the abbreviation “CS” and “CS-patient” as used herein, refer to patients which show either a severe course or a critical course of COVID-19 as described above.

The term “biomarker” or “biological marker” as used herein, is defined as a measurable indicator of a biological state or condition. Biomarker can be proteins, peptides, hormones, n- and o-glycans and other molecules, which show a biological state or condition of a subject. In the specific case of this invention only proteins (with the exception of CD15, which is a tetrasaccharide carbohydrate) were measured.

To assess the quality of the biomarkers a Quality score (“QS” or “Qual Score”) was calculated for each biomarker taking into account log FC values, adjusted p-values and coherent regulation in the different phases of the disease. For a quality assessment of the biomarkers stringent log FC-thresholds and adjusted p-values were used to attribute “points” to each biomarker. For example, in one embodiment +1 “point” was attributed to biomarkers having a |log FC|>1.0; another +1 “point” was attributed for |log FC|>1.5; another +1 “point” was attributed for |log FC|>2; another +1 “point” was attributed for |log FC|>3. In case of the adjusted p-value, +1 “point” was attributed to biomarkers having a p<0.003; another +1 “point” was attributed to biomarkers having a p<0.0001; another +1 “point” was attributed to biomarkers having a p<0.00001; another +1 “point” was attributed to biomarkers having a p<0.0001; another +1 “point” was attributed to biomarkers being significant differential between CS- and MM-patients in early as well as medium phase; another +1 “point” was attributed to biomarkers being significant differential between CS- and MM-patients in early, medium as well as late phase.

### Determining the Amount of the Biomarker

Determining the amount of the biomarkers referred to in this specification relates to measuring the amount or concentration, preferably semi-quantitatively or quantitatively. Measuring can be done directly or indirectly. Direct measuring relates to measuring the amount or concentration of the biomarker based on a signal that is obtained from the biomarker itself and the intensity of which directly or indirectly correlates with the number of molecules of the polypeptide present in the sample. Such a signal—sometimes referred to herein as intensity signal may be obtained, e.g., by measuring an intensity value of a specific physical or chemical property of the polypeptide. Indirect measuring includes measuring of a signal obtained from a secondary component (i.e. a component not being the biomarker itself) or a biological read out system, e.g., measurable cellular responses, ligands, labels, or enzymatic reaction products.

In accordance with the present invention, determining the amount of a biomarker can be achieved by different means for determining the amount of a biomarker in a sample. Said means comprise immunoassay devices and methods that may utilize labeled molecules in various sandwich, competition, or other assay formats. The immunoassay device may be an antibody array, in particular a planar antibody microarray or a bead based antibody microarray. Preferably quick assays, such as ELISA, lateral-flow-device, and/or multiplex lateral-flow-device. Also preferred are stripe tests. Said assays will develop a signal which is indicative for the presence or absence of the biomarker, e.g. a polypeptide biomarker.

Moreover, the signal strength can, preferably, be correlated directly or indirectly (e.g. proportional, or reverse-proportional) to the amount of biomarker present in a sample. Further suitable methods comprise measuring a physical or chemical property specific for the biomarker such as its precise molecular mass or NMR spectrum. Said methods comprise, preferably, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays, CBA (an enzymatic Cobalt Binding Assay), or latex agglutination assays. The determination of the amount of a biomarker can be performed in a medical laboratory or it can consist of a point-of-care testing.

Also preferably, determining the amount of a biomarker may comprise the step of measuring a specific intensity signal obtainable from the biomarker in the sample. As described above, such a signal may be the signal intensity observed at a mass to charge (m/z) variable specific for the biomarker observed in mass spectra or an NMR spectrum specific for the biomarker.

Determining the amount of a biomarker may, preferably, comprise the steps of
a) contacting the biomarker with a specific ligand,
b) optionally, removing non-bound ligand, and
c) measuring the amount of bound ligand.

The bound ligand will generate an intensity signal. Binding includes both covalent and non-covalent binding. A ligand can be any compound, e.g., a peptide, polypeptide, nucleic acid, or small molecule, binding to the biomarker described herein. Preferred ligands include antibodies, nucleic acids, peptides or polypeptides such as receptors or binding partners for the biomarker and fragments thereof comprising the binding domains for the peptides, and aptamers, e.g. nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g. phage display. Antibodies as referred to herein include both polyclonal and monoclonal antibodies, as well as fragments thereof, such as Fv, Fab, scFv and F(ab)2 fragments that are capable of binding antigen or hapten. The present invention also includes single chain antibodies and humanized hybrid antibodies wherein amino acid sequences of a non-human donor antibody exhibiting a desired antigen-specificity are combined with sequences of a human acceptor antibody. Alternatively, chimeric mouse antibodies with rabbit Fc can be used. The donor sequences will usually include at least the antigen-binding amino acid residues of the donor but may comprise other structurally and/or functionally relevant amino acid residues of the donor antibody as well. Such hybrids can be prepared by several methods well known in the art. Preferably, the ligand or agent binds specifically to the biomarker.

“Specific binding” according to the present invention means that the ligand or agent should not bind substantially to (“cross-react” with) another biomarker, polypeptide or substance present in the sample to be analyzed. Preferably, the specifically bound biomarker should be bound with at least 3 times higher, more preferably at least 10 times higher and even more preferably at least 50 times higher affinity than any other substance, biomarker or polypeptide in the sample. Nonspecific binding may be tolerable, if it can still be distinguished and measured unequivocally, e.g. according to its size on a Western Blot, or by its relatively higher abundance in the sample.

Binding of the ligand can be measured by any method known in the art. Preferably, said method is semi-quantitative or quantitative. Suitable methods are described in the following. First, binding of a ligand may be measured directly, e.g. by mass spectroscopy, NMR or surface plasmon resonance. Second, if the ligand also serves as a substrate of an enzymatic activity of the biomarker of interest, an enzymatic reaction product may be measured (e.g. the amount of a protease can be measured by measuring the amount of cleaved substrate, e.g. on a Western Blot). Alternatively, the ligand may exhibit enzymatic properties itself and the “ligand/biomarker” complex or the ligand that was bound by the biomarker, respectively, may be contacted with a suitable substrate allowing detection by the generation of an intensity signal.

For measurement of enzymatic reaction products, preferably the amount of substrate is saturating. The substrate may also be labeled with a detectable label prior to the reaction. Preferably, the sample is contacted with the substrate for an adequate period of time. An adequate period of time refers to the time necessary for a detectable, preferably measurable, amount of product to be produced. Instead of measuring the amount of product, the time necessary for appearance of a given (e.g. detectable) amount of product can be measured. Third, the ligand may be coupled covalently or non-covalently to a label allowing detection and measurement of the ligand.

Labeling may be done by direct or indirect methods. Direct labeling involves coupling of the label directly (covalently or non-covalently) to the ligand. Indirect labeling involves binding (covalently or non-covalently) of a secondary ligand to the first ligand. The secondary ligand should specifically bind to the first ligand. Said secondary ligand may be coupled with a suitable label and/or be the target (receptor) of a tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.).

The ligand or substrate may also be “tagged” with one or more tags. Such tags may then be targets for higher order ligands. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium, enzymatically active labels, radioactive labels, magnetic labels (“e.g. magnetic beads”, including paramagnetic and superparamagnetic labels), and fluorescent labels. Enzymatically active labels include e.g. horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star™ (Amersham Biosciences), ECF™ (Amersham Biosciences). A suitable enzyme-substrate combination may result in a colored reaction product, fluorescence or chemo luminescence, which can be measured according to methods known in the art (e.g. using a light-sensitive film or a suitable camera system). As for measuring the enzymatic reaction, the criteria given above apply analogously.

Suitable fluorescent labels include fluorescent proteins (such as GFP and its derivatives), Cy3, Cy5, or Dy-547, Dy-549, Dy-647, Dy-649 (Dyomics, Jena, Germany) or Texas Red, Fluorescein, and the Alexa dyes (e.g. Alexa 568). Further fluorescent labels are available e.g. from Molecular Probes (Oregon). Further, the use of quantum dots as fluorescent labels is contemplated. Suitable radioactive labels include <35>S, <125>I, <32>P, <33>P and the like. A radioactive label can be detected by any method known and appropriate, e.g. a light-sensitive film or a phosphor imager. Suitable measurement methods include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA), scintillation proximity assay (SPA), FRET based proximity assays (Anal Chem. 2005 Apr. 15; 77(8):2637-42.) or Ligation proximity assays (Nature Biotechnology 20, 473-477 (2002), turbidimetry, nephelometry, latex-enhanced turbidimetry or nephelometry, or solid phase immune tests. Further methods such as gel electrophoresis, 2D gel electrophoresis, SDS polyacrylamide gel electrophoresis (SDS-PAGE), Western Blotting, and mass spectrometry can be used alone or in combination with labeling or other detection methods as described above.

The amount of a biomarker may be, also preferably, determined as follows:
1) contacting a solid support comprising a ligand for the biomarker as specified above with a sample comprising the biomarker, and
2) optionally, removing non-bound biomarker may be done, and
3) measuring the amount of biomarker which is bound to the support.

The ligand, preferably, chosen from the group consisting of nucleic acids, peptides, polypeptides, antibodies and aptamers, is preferably present on a solid support in immobilized form.

Materials for manufacturing solid supports are well known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes, or combinations thereof.

The ligand or agent may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention.

Suitable methods for fixing/immobilizing said ligand include, but are not limited to ionic, hydrophobic, covalent interactions and the like. It is also contemplated to use “suspension arrays” as arrays according to the present invention (Nolan 2002, Trends Biotechnol. 20(l):9-12). In such suspension arrays, the carrier, e.g. a microbead or microsphere, is present in suspension. The array consists of different microbeads or microspheres, possibly labeled, carrying different ligands. Methods of producing such arrays, for example based on solid-phase chemistry and photolabile protective groups, are disclosed in U.S. Pat. No. 5,744,305, which is incorporated by reference as if fully set forth herein.

The term “amount” as used herein encompasses the absolute amount of a biomarker, the relative amount or concentration of the said biomarker as well as any value or parameter which correlates thereto or can be derived therefrom. Such values or parameters comprise intensity signal values from all specific physical or chemical properties obtained from the said biomarker by direct measurements, e.g., intensity values in mass spectra or NMR spectra or surface Plasmon resonance spectra. Moreover, encompassed are all values or parameters which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations.

The term “comparing” as used herein encompasses comparing the amount of the biomarker comprised in the sample to be analyzed with an amount of a suitable reference source specified else—where in this description. It is to be understood that “comparing” as used herein refers to a comparison of corresponding parameters or values, e.g., an absolute amount is compared to an absolute reference amount, while a concentration is compared to a reference concentration, or an intensity signal obtained from a test sample is compared to the same type of intensity signal of a reference sample. Preferably, the reference amount is the amount of the biomarker in healthy subjects, e.g. the average amount of the respective biomarker in a group of 10 or more, 30 or more, 50 or more, or 100 or more healthy subjects.

The comparison of the method of the present invention may be carried out manually or computer assisted. For a computer assisted comparison, the value of the determined amount may be compared to values corresponding to suitable references, which are stored in a database by a computer program. The computer program may further evaluate the result of the comparison, i.e. automatically provide the desired assessment in a suitable output format. Based on the comparison of the amount determined in step a) and the reference amount, it is possible to differentiate between the predisposition for a severe or critical course of a COVID-19-disease and a mild or moderate course of a COVID-19-disease in a subject.

The term “reference” as used herein refers to amounts of the biomarker which allow for predicting whether a subject is at risk for the predisposition for a severe or critical course of a COVID19-disease at an early time point. Therefore, the reference may be derived from a subject known to not being predisposed for a severe or critical course of a COVID-19-disease. In a preferred embodiment the reference or reference sample is taken from a subject with a mild or moderate course of a COVID-19-disease.

More preferably, an elevated amount of the said at least one biomarker selected from the biomarkers according to SEQ ID No. 1-5, 7-11, 13-15, 17-25, 27-35, 37-45, 48-50, 52-53, 56-58, 60-62 compared to the reference is indicative for a subject being at risk of being predisposed for a severe or critical course of a COVID-19-disease, whereas a lowered amount of the said at least one biomarker selected from the biomarkers according to SEQ ID No. 6, 12, 16, 26, 36, 46-47, 51, 54-55, 59 compared to the reference is indicative for a subject being at risk of being predisposed for a severe or critical course of a COVID-19-disease.

More preferably, an elevated amount of the said at least one biomarker selected from the biomarkers according to SEQ ID No. 106-143, 150-152 compared to the reference is indicative for a subject being at risk of being predisposed for a severe or critical course of a COVID-19-disease, whereas a lowered amount of the said at least one biomarker selected from the biomarkers according to SEQ ID No. 144-149 compared to the reference is indicative for a subject being at risk of being predisposed for a severe or critical course of a COVID-19-disease.

Preferably, the changes as referred to herein are statistically significant.

In the context of the methods of the present invention, the amount of more than one biomarker may be determined. Of course, the determined amounts may be compared to various reference amounts, i.e. to the reference amounts for the individual biomarker tested.

Moreover, the references, preferably, define threshold amounts or thresholds. Suitable reference amounts or threshold amounts may be determined by the method of the present invention from a reference sample to be analyzed together, i.e. simultaneously or subsequently, with the test sample. A preferred reference amount serving as a threshold may be derived from the upper limit of normal (ULN), i.e. the upper limit of the physiological amount to be found in a population of subjects (e.g. patients enrolled for a clinical trial). The ULN for a given population of subjects can be determined by well-known techniques. A suitable technique may be to determine the medium of the population for the biomarker amounts to be determined in the method of the present invention.

Suitable threshold amounts can also be identified by ROC plots depicting the overlap between the two distributions by plotting the sensitivity versus 1−specificity for the complete range of decision thresholds. On the y-axis is sensitivity, or the true-positive fraction, defined as (number of true-positive test results)/(number of true-positive+number of false-negative test results). This has also been referred to as positivity in the presence of a given disease. It is calculated solely from the affected sub-group. On the x-axis is the false-positive fraction, or 1−specificity, defined as (number of false-positive results)/(number of true-negative+number of false-positive results). It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/1-specificity pair corresponding to a particular decision threshold.

A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45 degrees diagonal line from the lower left corner to the upper right corner. Most plots fall in between these two extremes.

From the ROC plots the area under the curve (AUC) may be calculated, that is, AUC measures the entire two-dimensional area underneath the entire ROC curve (integral) from (0.0) to (1.1).

In machine learning the ROC AUC can be used as statistic for model comparison. The coherence of AUC as a measure of aggregated classification performance has been vindicated in terms of a uniform rate distribution and AUC has been linked to a number of other performance metrics such as the Brier score.

In this application, for each biomarker, both an individual AUC is calculated, as well as an AUC averaged over all biomarker combinations containing that biomarker, in order to provide a further parameter which allows the quantitative and/or qualitative appreciation of a biomarker.

### Preferred Groups of Biomarkers

The biomarkers as used herein include a polypeptide according to SEQ ID No. 1 to 152 or fragments or variants of such polypeptides (or, in case of CD15, a tetrasaccharide carbohydrate of formula I) for differentiation of the predisposition for a severe or critical course of a COVID-19-disease from a mild course of a COVID-19-disease in a subject.

“Polypeptide” and “protein” are used interchangeably herein. In the tables, all protein biomarkers are uniquely described by the Uniprot entry name, the Uniprot accession number as well as the respective gene name and official protein name as provided by the Uniprot database. For more information on the protein, see the UniProt Database, in particular, the UniProt release UniProt release 2020_03, published Jun. 17, 2020, see also The UniProt Consortium (2017). The sequences of all protein biomarkers of the invention are listed in the sequence listing under SEQ ID No. 1 to 152 and CD15, which is a tetrasaccharide carbohydrate of formula I.

Variants and/or isoforms of the biomarkers disclosed herein include polypeptides which differ in their amino acid sequences, e.g. due to the presence of conservative amino acid substitutions. Preferably, such variants and/or isoforms have an amino acid sequence being at least 70%, at least 80%, at least 90%, at least 95%, at least 98%, or at least 99% identical over the entire sequence region to the amino acid sequences of the aforementioned specific polypeptides given in the sequence listing. Variants may be allelic variants, splice variants or any other species specific homologs, paralogs, or orthologs. Preferably, the percent identity can be determined by the algorithms of Needleman and Wunsch or Smith and Waterman. Programs and algorithms to carry out sequence alignments are well known by a skilled artisan. To carry out the sequence alignments, the program PileUp (J. Mol. Evolution., 25, 351-360, 1987, Higgins et al., CABIOS, 5 1989: 151-153) or the programs Gap and BestFit (Needleman 1970, J. Mol. Biol. 48; 443-453 and Smith 1981, Adv. Appl. Math. 2; 482-489), which are part of the GCG software packet (Genetics Computer Group, 575 Science Drive, Madison, Wisconsin, USA 53711, Version 1991), may be used. The sequence identity values recited above in percent (%) may be determined using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments. In an embodiment, the variants of biomarkers include any isoforms of the respective biomarker.

In one embodiment the invention also encompasses all isoforms, fragments and variants of the canonical proteins as listed in Uniprot under the accession number in the section “similar proteins”. This section provides links to proteins that are similar to the protein sequence(s) described in this entry at different levels of sequence identity thresholds (100%, 90% and 50%) based on their membership in UniProt Reference Clusters (UniRef).

In one embodiment the biomarkers of the present invention are differentiated depending on their amount as compared to a reference amount. The amount of the biomarker may be either significantly upregulated (upregulated) or downregulated (downregulated). “Significant” in this respect refers to an adjusted p-value of less than 0.05 (adjusted for multiple testing according to Bonferroni and Hochberg).

In another embodiment the biomarkers of the present invention are differentiated depending on the time-point of their upregulation and/or downregulation. As such the present application differentiates between “acute phase”, i.e. from 3 days before until up to 9 days after onset of symptoms and/or a positive test for a SARS-CoV-2 infection, “medium phase”, i.e. between 10 to 21 days after onset of symptoms and/or a positive test for a SARS-CoV-2 infection; and “late phase”, i.e. more than 21 days after onset of symptoms and/or a positive test for a SARS-CoV-2 infection.

In yet another embodiment the biomarkers of the present invention are differentiated depending on the type of immune response to which it is associated. In general immune responses against pathogens are divided roughly into three types:

### Biomarker-Groups

In one embodiment, the one or more biomarker for differentiation of the predisposition for a severe or critical course of a COVID-19-disease from a mild or moderate course of a COVID-19-disease in a subject is selected from the group consisting of FGF2, CD28, TGFB2, 113R1, IL15, IGKC, I13R2, CSF1, IGF1R, BCAM, CD166, OX2G, CD45RA, TNR16, CXCR5, CCL19, LEUK, ICAM1, TNFL4, GLPB, IL2, PD1 L1, CCL27, IL3, HMGB1, ALBU, SLAF1, CD47, TNFA, TLR3, TBB3, S10A8/9, IL15, TNF11, TFR1, TNR8, CEAM1/3/5/6/8, AREG, HLA-1, CD81, VEGFA, CCL8, IL31, K1C18, IL12B, ERBB2, DPP4, CD45RB, CD8A, IL1A, HAVR2, IGLC1, TNFL6, CCL2, TNR5, ADIPO, ICAM1, CD4, CD38, and IL20, as well as combinations thereof and/or isoforms, fragments and/or variants thereof.

### Biomarkers of the “Quality Score>=4 Group”

36 biomarkers show a “quality score” of five or higher and are therefore preferred for determination whether a subject has a predisposition for a severe or critical course of a COVID-19-disease according to said “quality score” are TNR8, CCL8, TNF11, ICAM1, TBB3, AREG, TNFL4, IL3, CD45RB, CEAM1/3/5/6/8, SLAF1, TNFA, CCL19, IGKC, HMGB1, CCL27, CD47, CD45RA, GLPB, LEUK, PD1 L1, TNR16, IL2, TLR3, CXCR5, OX2G, I13R2, BCAM, IGF1R, CSF1, TGFB2, FGF2, CD166, 1L15, CD28, and I13R1 as well as combinations thereof and/or isoforms, fragments and/or variants thereof.

### Biomarkers of the “Quality Score>=5 Group”

24 biomarkers show a “quality score” of five or higher and are therefore preferred for determination whether a subject has a predisposition for a severe or critical course of a COVID-19-disease according to said “quality score” are CCL19, IGKC, HMGB1, CCL27, CD47, CD45RA, GLPB, LEUK, PD1 L1, TNR16, IL2, TLR3, CXCR5, OX2G, I13R2, BCAM, IGF1R, CSF1, TGFB2, FGF2, CD166, 1L15, CD28, and I13R1 as well as combinations thereof and/or isoforms, fragments and/or variants thereof.

### Biomarkers of the “Quality Score>=6 Group”

16 biomarkers show a “quality score” of six or higher and are therefore preferred for determination whether a subject has a predisposition for a severe or critical course of a COVID-19-disease according to said “quality score” are PD1L1, TNR16, IL2, TLR3, CXCR5, OX2G, I13R2, BCAM, IGF1R, CSF1, TGFB2, FGF2, CD166, 1L15, CD28, and I13R1 as well as combinations thereof and/or isoforms, fragments and/or variants thereof.

### Biomarkers of the “Quality Score>=8 Group”

Five biomarkers show a “quality score” of eight or higher and are therefore preferred for determination whether a subject has a predisposition for a severe or critical course of a COVID-19-disease according to said “quality score” are I13R1, IL15, FGF2, CD28, and CD166 as well as combinations thereof and/or isoforms, fragments and/or variants thereof.

In case of a test-development, these biomarkers are preferred, since they allow a significant differentiation of the predisposition for a severe or critical course of a COVID-19-disease from a mild or moderate course of a COVID-19-disease in a subject.

In further embodiments the quality score of the biomarkers may be adapted to the exact problem.

For example a biomarker suitable for the early identification of the predisposition for a severe or critical course of a COVID-19-disease in a subject shows significant increase or decrease during acute phase.

### Biomarkers of the “Acute Group”

The following biomarkers of the “acute group” are significantly upregulated in CS-patients during acute phase of the disease: TNFA, SLAF1, TNFL4, CD8A, IL20, ERBB2, CD38, as well as isoforms, fragments and/or variants thereof (see also FIG. 1 and table 2). The following biomarkers of the “acute group” are significantly downregulated in CS-patients during acute phase of the disease: ALBU, HAVR2, ADIPO, DPP4 and IGLC1, as well as isoforms, fragments and/or variants thereof (see also FIG. 1 and table 2).

These biomarkers are preferred to identify early the predisposition of a severe or critical course of a COVID-19-disease.

In one embodiment the following biomarkers are preferred in this group: SLAF1, CD8A, ERBB2, ALBU and/or IGLC1.

### Biomarkers of the “Acute and Medium Group”

The following biomarkers of the “acute and medium group” are significantly upregulated both during acute and medium phase in a CS-patient: LEUK, GLPB, CD45RB, CD81, K1C18, CEAM1,3,5,6,8, VEGFA, IL3, IL12B, S10A8/9, HLA-1, CD45RA, TNFL6, as well as isoforms, fragments and/or variants thereof (see also FIG. 1 and table 2).

These biomarkers may be used additionally to identify early the predisposition of a severe or critical course of a COVID-19-disease.

In one embodiment the following biomarkers are preferred in this group: CD81, CEAM1,3,5,6,8 and/or S10A8/9.

### Biomarkers of the “Acute, Medium and Late Group”

The following biomarkers of the “acute, medium and late group” are significantly upregulated during acute, medium and late phase in a CS-patient: CXCR5, CD28, IL15, IGF1R, IL2, TGFB2, TNR16, TLR3, FGF2, CD4, CSF1, 113R1, TBB3, BCAM, CD166, PD1L2, HMGB1, CD47, 113R2, CCL27, AREG, IL1A, TNF11, as well as isoforms, fragments and/or variants thereof (see also FIG. 1 and table 2). The following biomarkers of the “acute, medium and late group” are significantly downregulated during acute, medium and late phase in a CS-patient: OX2G, CCL8, IL31, and CCL19, as well as isoforms, fragments and/or variants thereof (see also FIG. 1 and table 2).

Since these biomarkers are de-regulated during the whole course of the COVID-19-disease, they are preferably used both to identify early the predisposition of a severe or critical course of a COVID-19-disease as well as to monitor the further development of the disease in the patient.

### Biomarkers of the “Medium Group”

The following biomarkers of the “medium group” are significantly upregulated only during medium phase in a CS-patient: OSTP, INHBA, HGF, TR13B, CTLA4, and CD276, as well as isoforms, fragments and/or variants thereof (see also FIG. 1 and table 1). The following biomarkers of the “medium group” are significantly downregulated only during medium phase in a CS-patient: TNFB, CO3, IFNG, CCL28, as well as isoforms, fragments and/or variants thereof (see also FIG. 1 and table 3).

Since these biomarkers are de-regulated during the middle of the COVID-19-disease, they may be used to monitor the further development of the disease in the patient.

### Biomarkers of the “Medium and Late Group”

The following biomarkers of the “medium and late group” are significantly upregulated both during medium and late phase in a CS-patient: TNR6, NCAM1, CD3deg, BTLA, IFNL2, ANGP4, and SLAF8, as well as isoforms, fragments and/or variants thereof. The following biomarkers of the “medium and late group” are significantly downregulated both medium and late phase in a CS-patient: AMPN, TSLP, CXL13, CCL7, HLA-class II, as well as isoforms, fragments and/or variants thereof (see also FIG. 1 and table 4).

Since these biomarkers are de-regulated during the middle of the COVID-19-disease as well as at the end, they may be used to monitor the further development of the disease in the patient as well as the recovery of the patient.

### Biomarkers of the “Late Group”

The following biomarkers of the “late group” are significantly upregulated only during late phase in a CS-patient: TR11B, CD15, IL22, IL2RA, FLT3, PLF4, PRIO, LIF, ITAM, CD72, NP1L4, ONCM, ITA2B, and HLA-ABC as well as isoforms, fragments and/or variants thereof. The following biomarkers of the “late group” are significantly downregulated only during late phase in a CS-patient: AGRE5, EGLN, CXCL9, SCRB2, CCL3 and SLAF1, as well as isoforms, fragments and/or variants thereof (see also FIG. 1 and table 5).

Since these biomarkers are de-regulated during the late course of the COVID-19-disease, they may be to monitor the further development of the disease and recovery in the patient.

### Biomarkers of the “Immune Cell Activation”

The following biomarkers of the “immune cell activation” are significantly upregulated in CS-patients: CCL2, CD4, CD28, CD38, CD47, CD81, CEAM1, CSF1, ERBB2, HMGB1, IL2, IL12B, IL15, 113R2, LEUK, PD1 L1, PTPRC, TFR1, TNFL4, TNF11, and TNR5 as well as combinations thereof and isoforms, fragments and/or variants thereof. The following biomarkers are significantly downregulated in CS-patients: CCL19, DPP4, HAVR2, and OX2G, as well as combinations thereof and isoforms, fragments and/or variants thereof.

These biomarkers play a role in the immune cell activation. Thus, without being bound to theory, a disturbance in these processes may foster a severe or critical course of a COVID-19-disease.

In one embodiment the following biomarkers are preferred in this group: CCL2, CD28, CD47, CD81, 113R2 and/or ERBB2.

### Biomarkers of the “Cytokine Production and Signaling Group”

The following biomarkers of the “cytokine production and signaling” are significantly upregulated in CS-patients: CCL2, CD4, CD28, CEAM1, CSF1, CXCR5, FGF2, HMGB1, ICAM1, IL1A, IL2, IL3, IL12B, IL15, IL20, 113R1, 113R2, LEUK, K1C18, PD1L1, SLAF1, TGFB2, TNFA, TNFL4, TNFL6, TNF11, TNR5, TNR16, TLR3, and VEGFA, as well as combinations thereof and isoforms, fragments and/or variants thereof. The following biomarkers are significantly downregulated in CS-patients: ADIPO, CCL8, CCL19, HAVR2, IL31, and TNR8, as well as combinations thereof and isoforms, fragments and/or variants thereof.

These biomarkers play an important role in cytokine production and signaling. The deregulation of cytokines is one of the reasons for the Covid-19 induced cytokine release syndrome “CRS”.

Thus, without being bound to theory, a disturbance in these processes may foster a severe or critical course of a COVID-19-disease.

In one embodiment the following biomarkers are preferred in this group: CCL2, CXCR5, FGF2, 113R2, SLAF1, and/or TNR16.

### Biomarkers of the “RNA Metabolism Group”

The following biomarkers of the “positive regulation of cytokine production group” are significantly upregulated in CS-patients: CD4, CD28, CD38, CD81, ERBB2, FGF2, HMGB1, ICAM1, IL1A, IL2, TNR16, TGFB2, TLR3, TNFA, TNFL4, TNFL6, TNF11, TNR5, and VEGFA, as well as combinations thereof and isoforms, fragments and/or variants thereof. The following biomarkers are significantly downregulated in CS-patients: ADIPO, and HAVR2, as well as combinations thereof and isoforms, fragments and/or variants thereof.

These biomarkers play a role in RNA metabolism and may have a direct impact on viral replication. Thus, without being bound to theory, a disturbance in these processes may foster a severe or critical course of a COVID-19-disease and may be an early indicator of a developing CRS in a patient.

### Biomarkers of the “NF-Kappa B (NF-κB) Group”

The following biomarkers of the “negative regulation of immune system process group” are significantly upregulated in CS-patients: CD4, TNR5, TNFL6, TLR3, TNFA, and TNF11, as well as combinations thereof and isoforms, fragments and/or variants thereof. The following biomarkers are significantly downregulated in CS-patients: ADIPO, and CCL19, as well as combinations thereof and isoforms, fragments and/or variants thereof.

These biomarkers are known to play a role in NF-kappa B (NF-κB) signaling. NF-κB (nuclear factor kappa-light-chain-enhancer of activated B cells) is a protein complex that controls transcription of DNA, cytokine production and cell survival. NF-κB is found in almost all animal cell types and is involved in cellular responses to stimuli such as stress, cytokines, free radicals, heavy metals, ultraviolet irradiation, oxidized LDL, and bacterial or viral antigens. NF-κB plays a key role in regulating the immune response to infection. Incorrect regulation of NF-κB has been linked to cancer, inflammatory and autoimmune diseases, septic shock, viral infection, and improper immune development. NF-κB has also been implicated in processes of synaptic plasticity and memory. Thus, without being bound to theory, a disturbance in these processes may foster a severe or critical course of a COVID-19-disease.

### Biomarkers of the “MAPK/ERK Signaling Group”

The following biomarkers of the “positive regulation of MAPK cascade group” are significantly upregulated in CS-patients: CCL2, CD4, CEAM1, CD81, ERBB2, FGF2, HMGB1, ICAM1, SLAF1, TGFB2, TLR3, TNFA, TNF11, TNR5, and VEGFA, as well as combinations thereof and isoforms, fragments and/or variants thereof. The following biomarkers are significantly downregulated in CS-patients: ADIPO, CCL8, CCL19, and HAVR2 as well as combinations thereof and isoforms, fragments and/or variants thereof.

These biomarkers are known to play a role in regulation of the MAPK cascade. The MAPK-pathway is thought to play an important role in immune-regulation, especially the regulation of t-cell development and other inflammatory processes. Thus, without being bound to theory, any disturbance in these processes may foster a severe or critical course of a COVID-19-disease.

Furthermore, the intracellular Raf/MEK/ERK signaling pathway is central for replication of many RNA viruses, such as the influenza virus, Hantavirus or respiratory syncytial virus (RSV) and also SARS-CoV-2, the virus that causes COVID-19. Thus, without being bound to theory, the disturbance of the MAPK pathway may increase the export of the viral genome protein complexes (ribonucleoprotein, RNP) from the nucleus to the cytoplasm, thus enhancing the formation of functional new viral particles. This ultimately increases the viral load in the body and may lead to more severe or critical courses of the COVID-19 disease in some patients.

In one embodiment the following biomarkers are preferred in this group: CCL2, CD81, ERBB2, FGF2, SLAF1, VEGFA.

In further studies protein interaction of de-regulated biomarkers were identified, which reveal several direct as well as indirect interactions of the differential proteins within acute CS-patients:

### Biomarkers of the “CD4 Group”

The following biomarkers of the “CD4 group” are significantly de-regulated in CS-patients: IL20, IL12B, I13R1, IL15, IL3, IL2, 113R2, CD8A, CD28, CD4, PD1 L1, TFR1, CD45RA, CD45RB, LEUK, and SLAF1, as well as combinations thereof and isoforms, fragments and/or variants thereof.

These biomarkers are all linked to important pathways of the immune-response, especially in cell-cell interactions within the immune system. Thus, without being bound to theory, any disturbance in these processes may foster a severe or critical course of a COVID-19-disease.

### Biomarkers of the “CD47 Group”

The following biomarkers of the “CD47 group” are significantly de-regulated in CS-patients: CD47, CEAM1/3/5/6/8, and HAVR2, as well as combinations thereof and isoforms, fragments and/or variants thereof.

These biomarkers are all cell surface-proteins related to myeloid cell activation and regulation of T cell activation. Thus, without being bound to theory, a disturbance in these processes may foster a severe or critical course of a COVID-19-disease.

### Biomarkers of the “CXCR5 Group”

The following biomarkers of the “CXCR5 group” are significantly de-regulated in CS-patients: CCL27, CCL19, CCL2, CCL8 and CXCR5, as well as combinations thereof and isoforms, fragments and/or variants thereof.

These biomarkers are chemokines and a chemokine receptor involved in leukocyte chemotaxis and humoral immune response as well as T-cell activation via MAPK cascade. Thus, without being bound to theory, a disturbance in these processes may foster a severe or critical course of a COVID-19-disease.

### Biomarkers of the “ERBB2 Group”

The following biomarkers of the “ERBB2 group” are significantly de-regulated in CS-patients: ICAM1, AREG, FGF2, IGF1R, ERBB2, TNR16, VEGFA, TGFB2, ALBU, CSF1, HLA-I (HLAB), TNFA, and TNF11, as well as combinations thereof and isoforms, fragments and/or variants thereof.

These biomarkers are involved in positive regulation of cell migration and stress-activated MAPK cascade. Thus, without being bound to theory, a disturbance in these processes may foster a severe or critical course of a COVID-19-disease.

In one embodiment the following biomarkers are preferred in this group: AREG, FGF2, IGF1R, ERBB2, TNR16 and/or ALBU.

### Biomarker-Clusters

The cluster-analysis (see example 3) revealed that certain biomarkers have a common profile in the sample cohort and show similar de-regulation in most CS-patient samples. This can imply that they are functionally related to each other. More importantly, the protein clusters describe proteins with a similar abundance profile in the sample set. Thereby, the individual proteins within each group exhibit a similar de-regulation and thereby are redundant for discrimination of the samples.

This also means that biomarkers from different clusters are much less related in their functionality. Still, as shown in this application, they show a similar de-regulation in CS-patients as compared to MM-patients. Thus, a signal received by the de-regulation of at least two biomarkers from at least two different clusters is more robust (since they are more likely to show independent effects) in predicting a severe or critical course of the COVID-19-disease than for example a selection of at least two biomarkers from the same cluster.

Thus, in a preferred embodiment at least two biomarkers from at least two different cluster-groups are preferably selected for a potential biomarker test according to the present invention. In an even more preferred embodiment said at least two biomarkers are picked from the at least two different cluster-groups by picking the biomarkers with the highest quality score of each cluster.

The cluster-groups sharing a similar significant de-regulation in CS-patients are:

### Biomarkers of the “Cluster 1 Group”

The following biomarkers of the “cluster 1 group” are significantly de-regulated in CS-patients: HMGB1, (IL15*), HLA-1, AREG, CD81, CD47, IL1A, IL12B, TBB3, TNR16, S10A8/9, OX2G, CCL8, TNR8, DPP4, HAVR2, CCL19, IL31, ADIPO, ALBU, IGKC, and IGLC1, as well as combinations thereof and isoforms, fragments and/or variants thereof.

*IL15 was recognized in two clusters by two different antibodies. This may be due to unspecific binding of one antibody. IL15 belongs most likely to cluster 3 only. Thus, in one embodiment cluster 1-group encompasses preferably not IL15.

In one embodiment the following biomarkers are preferred in this group: AREG, CD81, S10A8/9, ALBU and/or IGLC1.

### Biomarkers of the “Sub-Cluster 1a Group”

The following biomarkers of the “sub-cluster 1a group” are significantly de-regulated in CS-patients: HMGB1, IL15, HLA-1, and AREG, as well as combinations thereof and isoforms, fragments and/or variants thereof.

### Biomarkers of the “Sub-Cluster 1b Group”

The following biomarkers of the “sub-cluster 1b group” are significantly de-regulated in CS-patients: CD81, CD47, IL1A, IL12B, TBB3, TNR16, and S10A8/9, as well as combinations thereof and isoforms, fragments and/or variants thereof.

Proteins in this group are involved in T cell activation, innate immune response, angiogenesis and immune effector process.

### Biomarkers of the “Sub-Cluster 1c Group”

The following biomarkers of the “sub-cluster 1c group” are significantly de-regulated in CS-patients: OX2G, CCL8, TNR8, DPP4, and HAVR2, as well as combinations thereof and isoforms, fragments and/or variants thereof.

Proteins in this heterogenous group are lower abundant in CS-patients. Proteins are active as T-cell co-stimulators as well as macrophage activation and attracting monocytes, basophils and eosinophils. Thus, without being bound to theory, a disturbance in these processes may foster a severe or critical course of a COVID-19-disease.

### Biomarkers of the “Sub-Cluster 1d Group”

The following biomarkers of the “sub-cluster 1d group” are significantly de-regulated in CS-patients: CCL19, IL31, ADIPO, ALBU, IGKC, and IGLC1, as well as combinations thereof and isoforms, fragments and/or variants thereof.

Proteins in this group are lower abundant in CS-patients.

### Biomarkers of the “Cluster 2 Group”

The following biomarkers of the “cluster 2 group” are significantly de-regulated in CS-patients: TLR3, CD166, CXCR5, CD45Ra, LEUK, GLPB, CD4, ICAM1, TFR1, TNF11, TNR5, ERBB2, IL20, CD38, IL3, TNFL4, CD8A, TNFL6, K1C18, SLAF1, FGF2, 113R1, IGF1R, I13R2, CSF1, CD45RB, PD1 L1, CCL2, TNFA, and CEAM1/3/5/6/8, as well as combinations thereof and isoforms, fragments and/or variants thereof.

The majority of the biomarkers found in this cluster is known to be involved in a positive regulation of the immune system process (GO:0002684) and exhibit cytokine (GO:0005125) or signaling receptor activity (GO:0038023). Thus, without being bound to theory, a disturbance in these processes may foster a severe or critical course of a COVID-19-disease.

In one embodiment the following biomarkers are preferred in this group: CXCR5, ERBB2, CD8A, SLAF1, FGF2, IGF1R, I13R2, CCL2 and/or CEAM1/3/5/6/8.

### Biomarkers of the “Sub-Cluster 2a Group”

The following biomarkers of the “sub-cluster 2a group” are significantly de-regulated in CS-patients: TLR3, CD166, CXCR5, CD45Ra, LEUK, and GLPB, as well as combinations thereof and isoforms, fragments and/or variants thereof.

All proteins found in this cluster are cellular surface proteins having a transmembrane helical domain and are involved in leukocyte or lymphocyte activation or migration. Most of the proteins found in this cluster are known to be involved in T-cell activation. Thus, without being bound to theory, a disturbance in these processes may foster a severe or critical course of a COVID-19-disease.

### Biomarkers of the “Sub-Cluster 2b Group”

The following biomarkers of the “sub-cluster 1b group” are significantly de-regulated in CS-patients: CD4, ICAM1, TFR1, TNF11, and TNR5, as well as combinations thereof and isoforms, fragments and/or variants thereof.

All proteins in this sub-cluster group are receptors involved in a cellular response to a cytokine stimulus and a positive regulation of the immune system. Majority of the proteins in this sub-cluster is involved in T-cell activation. Thus, without being bound to theory, a disturbance in these processes may foster a severe or critical course of a COVID-19-disease.

### Biomarkers of the “Sub-Cluster 2c Group”

ERBB2, IL20, CD38, IL3, TNFL4, CD8A, and TNFL6, as well as combinations thereof and isoforms, fragments and/or variants thereof.

All proteins in this sub-cluster are either involved in immune response or its regulation. Thus, without being bound to theory, a disturbance in these processes may foster a severe or critical course of a COVID-19-disease.

### Biomarkers of the “Sub-Cluster 2d Group”

The following biomarkers of the “sub-cluster 2d group” are significantly de-regulated in CS-patients: K1C18, SLAF1, FGF2, 113R1, IGF1R, I13R2, CSF1, CD45RB, PD1L1, CCL2, TNFA, and CEAM1/3/5/6/8, as well as combinations thereof and isoforms, fragments and/or variants thereof.

Proteins in this more heterogeneous sub-cluster are involved in a positive regulation of MAPK cascade, in a positive regulation of mononuclear cell proliferation and in an activation of myeloid leukocytes. Thus, without being bound to theory, a disturbance in these processes may foster a severe or critical course of a COVID-19-disease.

### Biomarkers of the “Cluster 3 Group”

The following biomarkers of the “cluster 3 group” are significantly de-regulated in CS-patients: TGFB2, IL2, VEGFA, BCAM, CD28, CCL27, IL15*, as well as combinations thereof and isoforms, fragments and/or variants thereof.

*IL15 was recognized in two clusters by two different antibodies. This may be due to unspecific binding of one antibody. IL15 belongs most likely to cluster 3 only. Thus, in one embodiment cluster 3-group encompasses preferably IL15.

Proteins in this cluster have a stimulating or activating effect on T-cells as well as in RNA metabolic processes. Thus, without being bound to theory, a disturbance in these processes may foster a severe or critical course of a COVID-19-disease.

Thus, in one embodiment preferred biomarker groups may be defined also as follows.

### Biomarkers of the “Best of Each Cluster Group”

In an embodiment the best performing biomarker (i.e. with highest quality score) from each main cluster are selected for a preferred group. The following biomarkers of the “best of each cluster group” are significantly de-regulated in CS-patients: I13R1, CD28 and OX2G, as well as combinations thereof and isoforms, fragments and/or variants thereof. These biomarkers are representatives of the highest quality score of each cluster and therefore are a preferred selection, since their signal is most likely not redundant.

### Biomarkers of the “Best Two of Each Cluster Group”

In an embodiment the best performing two biomarkers (i.e. with highest quality score) from each main cluster are selected for a preferred group. The following biomarkers of the “best of each cluster group” are significantly de-regulated in CS-patients: CD28, IL15, 113R1, FGF2, OX2G and TNR16, as well as combinations thereof and isoforms, fragments and/or variants thereof.

These biomarkers are two representatives of the highest quality score of each cluster and therefore are a preferred selection, since their signal is most likely not redundant.

### Biomarkers of the “Best of Each Cluster and Sub-Cluster Group”

In an embodiment the best performing biomarker (i.e. with highest quality score) from each main cluster and sub-cluster is selected for a preferred group. The following biomarkers of the “best of each cluster and sub-cluster group” are significantly de-regulated in CS-patients: CD28, 113R1, IL3, TNF11, CD166, CCL19, OX2G, TNR16, and HMBG1, as well as combinations thereof and isoforms, fragments and/or variants thereof.

Preferred biomarkers within this group are in some embodiments three biomarkers, comprising IL3 combined with two biomarkers of the following groups of two: CD28 and OX2G, TNR16 and OX2G, CD28 and CCL19, CD28 and CD166, TNF11 and I13R1, or TNF11 and TNR16.

These biomarkers are representatives of the highest quality score of each cluster and each sub-cluster and therefore are a preferred selection, since their signal is most likely not redundant.

### Biomarkers of the “Best of Cluster 1 Group”

In an embodiment the best performing biomarkers from each sub-clusters within main cluster 1 are selected for a preferred group. The following biomarkers of the “best of cluster 1” are significantly de-regulated in CS-patients: CCL19, OX2G, TNR16, and HMBG1, as well as combinations thereof and isoforms, fragments and/or variants thereof. These biomarkers are representatives of the highest quality score of cluster 1 and therefore are a preferred selection, since their signal is most likely not redundant to other clusters.

### Biomarkers of the “Best of Cluster 2 Group”

In an embodiment the best performing biomarker with a quality score of six or higher from main cluster 2 are selected for a preferred group. The following biomarkers of the “best of cluster 2” are significantly de-regulated in CS-patients: I13R1, FGF2, CD166, 113R2, IGF1R, CSF1, CXCR5, PD1 L1, and TLR3, as well as combinations thereof and isoforms, fragments and/or variants thereof. These biomarkers are representatives of the highest quality score of cluster 2 and therefore are a preferred selection, since their signal is most likely not redundant to other clusters.

### Biomarkers of the “Best of Sub-Cluster 2d Group”

In an embodiment the best performing biomarkers with a quality score of seven or higher from sub-cluster 2d are selected for a preferred group. The following biomarkers of the “best of cluster 2d” are significantly de-regulated in CS-patients: I13R1, FGF2, 113R2, IGF1R, and CSF1, as well as combinations thereof and isoforms, fragments and/or variants thereof. These biomarkers are representatives of the highest quality score of sub-cluster 2d and therefore are a preferred selection, since their signal is most likely not redundant to other clusters.

Further preferred biomarker-groups can be identified in example 2 and tables 2-4.

### Biomarkers of “Combined Regulation Clusters”

Some combinations of regulations clusters are preferred to improve the accuracy, sensitivity and/or sensibility of a diagnostic test.

The term “sensitivity” as used herein refers to a “true positive rate” and measures the proportion of positives that are correctly identified (i.e. the proportion of those who have some condition (affected) who are correctly identified as having the condition).

The term “specificity” as used herein refers to a “true negative rate” and measures the proportion of negatives that are correctly identified (i.e. the proportion of those who do not have the condition (unaffected) who are correctly identified as not having the condition).

The term “accuracy” as used herein refers to the overall accuracy of the detection

$$Accuracy = \frac{T P + T N}{T P + T N + F P + F N}$$

$$where T P = True posti ve ; F P = False positive ; \\ T N = True negative ; F N = False negative .$$

It has been surprisingly found that some of the groups are depicted in table 6 are of particular relevance when assessing the risk of a COVID-19 disease, namely the groups “positive regulation of cell population proliferation”, preferably the biomarkers AREG, CD28, CD47, CD81, ERBB2, FGF2, SLAF1; “positive regulation of cell adhesion”, preferably the biomarkers CCL2, CD28, CD47 and/or ERBB2; “positive regulation of lymphocyte activation”, preferably the biomarkers CCL2, CD28, CD47, and/or CD81; “negative regulation of response to stimulus”, preferably the biomarkers CCL2, ERBB2, FGF2, 113R2, and/or SLAF1; “positive regulation of MAPK cascade”, preferably the biomarkers CCL2, CD81, ERBB2, FGF2, and/or SLAF1; “cytokine-mediated signalling pathway”, preferably the biomarkers CCL2, CXCR5, FGF2, and/or TNR16; “regulation of leukocyte activation”, preferably the biomarkers CCL2, CD28, CD47, CD81, and/or ERBB2; “regulation of cell death”, preferably the biomarkers ALBU, CCL2, CD28, FGF2, and/or IGF1R; “regulation of T cell activation”, preferably the biomarkers CCL2, CD28, CD47 and/or ERBB2; “regulation of ERK1 and ERK2 cascade”, preferably the biomarkers CCL2, ERBB2, FGF2, and/or SLAF1; and/or “response to growth factor”, preferably the biomarkers CCL2, ERBB2, FGF2, and/or TNR16.

As becomes apparent some biomarkers repeatedly appear in those groups (such as ERBB2, CCL2, FGF2, CD28, CD81, SLAF1, CD47; even more preferably CCL2, ERBB2, FGF2 and SLAF1) and happen to improve the overall accuracy, sensitivity and/or sensibility of a diagnostic test significantly when combined in three and/or four biomarker-combinations. Thus, in one embodiment, at least three biomarkers and up to five biomarkers are selected from the above-mentioned groups.

### Further Preferred Biomarkers

It becomes apparent from the machine-learning analysis, that by combination of at least two, preferably three, more preferably four biomarkers and up to five, up to six, up to seven, up to eight or up to nine biomarkers, a high diagnostic certainty with respect to accuracy, sensitivity and/or sensibility can be reached. Usually, but depending on the specific case, more than 10 biomarkers are not used for diagnostics, since the tests becomes more costly and more complex with too many biomarkers, although additional biomarkers may improve the accuracy, sensitivity and/or sensibility even further.

The at least 2 and up to 9, preferably between 2 to 5 biomarkers, in one embodiment two biomarkers, in another embodiment three biomarkers, in another embodiment four biomarkers, may be selected preferably from biomarkers with a |log FC| of at least 0.5, preferably at least 0.8, preferably at least 1.0 as disclosed in this specification, in one embodiment the biomarkers are preferably selected from tables 16 and 17. In an additional embodiment the ROC AUC-value may be used to further rank the biomarkers within the biomarkers with a |log FC| of at least 0.5, in another embodiment the “quality score” may be used.

In yet a further embodiment these biomarkers can be combined with ERBB2, CD14, CCL2, UTER and/or CADH1 in order to improve the accuracy, sensitivity and/or sensibility even further.

Certain preferred biomarker-combinations can also be found in table 15.

### Single Description of the Most Preferred Biomarkers

Some biomarkers are very prominent in one or more aspect and therefore are preferably suitable as biomarkers for the identification of a predisposition for a severe or critical course of a COVID-19-disease in a subject.

In one embodiment FGF2 is a preferred biomarker, since it shows one of the highest quality scores in the measurements. It is upregulated throughout all three time-phases which were measured in patients with severe or critical courses of COVID-19. FGF2 acts as a ligand for FGFR1, FGFR2, FGFR3 and FGFR4. It also acts as an integrin ligand which is required for FGF2 signaling. It binds to integrin ITGAV:ITGB3 and plays an important role in the regulation of cell survival, cell division, cell differentiation and cell migration. It also functions as a potent mitogen e.g. fibroblasts in vitro and can induce angiogenesis. Last, but not least, it mediates phosphorylation of ERK1/2. The intracellular Raf/MEK/ERK signaling pathway is central immune regulation and inflammatory processes, thus it may lead to an upregulated secretion of cytokines, which may result in Covid-19 induced cytokine release syndrome “CRS”. Furthermore, there are indications that intracellular Raf/MEK/ERK signaling pathway may play a role also for replication of many RNA viruses, such as the influenza virus, Hantavirus or respiratory syncytial virus (RSV) and also SARS-CoV-2, the virus that causes COVID-19. Thus, without being bound to theory, the interaction of upregulated FGF2 with ERK kinases may increase the export of the viral genome protein complexes (ribonucleoprotein, RNP) from the nucleus to the cytoplasm, thus enhancing the formation of functional new viral particles. This ultimately increases the viral load in the body and may lead to more severe or critical courses of the COVID-19 disease in some patients.

In another embodiment TNR8 is a preferred biomarker, since it is the most downregulated biomarker during acute phase. TNR8 is a receptor for TNFSF8/CD30L. It plays a role in the regulation of cellular growth and transformation of activated lymphoblasts. It regulates gene expression through activation of NF-kappa-B. Thus, without being bound to theory, if TNR8 is downregulated, the transformation of activated lymphoblasts and T-cell proliferation, especially CD8+ T-cells may be negatively impacted, which prevents an adequate immune reaction, may lead to the finding of lymphopenia in severely ill patients and may lead to more severe or critical courses of the COVID-19 disease in some patients.

In yet another embodiment CD28 is a preferred biomarker, since it is the most upregulated biomarker during acute phase. CD28 is involved in T-cell activation, the induction of cell proliferation and cytokine production and promotion of T-cell survival. CD28 enhances the production of IL2, IL4 and IL10 in T-cells (which were reported to be upregulated in some patients as well in the course of Covid-19 induced cytokine release syndrome “CRS”) in conjunction with TCR/CD3 ligation and CD40L co-stimulation. Thus, without being bound to theory, an upregulation of CD28 may result in a CRS immune reaction, which results in a more severe or critical course of the COVID-19 disease in some patients

In yet another embodiment OX2G is a preferred biomarker, since it is the most downregulated biomarker both during medium and late phase. OX2G co-stimulates T-cell proliferation and may regulate myeloid cell activity in a variety of tissues. OX2G also negatively regulates macrophage activation and Nf-kappa B activation. Thus, without being bound to theory, a down-regulation may lead to enhanced inflammatory cytokine release and an activation of macrophage (“macrophage activation syndrome”) which was reported in severely ill Covid-19 patients.

In yet another embodiment CXCR5 is a preferred biomarker, since it is the most upregulated biomarker during medium phase. CXCR5 is a cytokine receptor that binds to B-lymphocyte chemoattractant (BLC). It is involved in B-cell migration into B-cell follicles of spleen and Peyer patches but not into those of mesenteric or peripheral lymph nodes. It may have a regulatory function in Burkitt lymphoma (BL) lymphomagenesis and/or B-cell differentiation. Thus, without being bound to theory, any disturbance in these processes may foster a severe or critical course of a COVID-19-disease. In yet another embodiment TLR3 is a preferred biomarker, since it is the most upregulated biomarker during late phase. TLR3 is a key component of innate and adaptive immunity. TLRs (Toll-like receptors) control host immune response against pathogens through recognition of molecular patterns specific to microorganisms. TLR3 is a nucleotide-sensing TLR which is activated by double-stranded RNA, a sign of viral infection. Acts via the adapter TRIF/TICAM1, leading to NF-kappa-B activation, IRF3 nuclear translocation, cytokine secretion and the inflammatory response. Thus, without being bound to theory, the NF-kappa-B-activation, and the upregulation of TLR3 may differentially enhance Ca²⁺ signaling, both may result in cytokine (over-)expression. Furthermore, Ca²⁺ potentiates cytokine release in hMSCs (human mesenchymal stem cells). All of these effects may result in an enhancement of a CRS-reaction (Covid-19 induced cytokine release syndrome) and lead to more severe or critical courses of the COVID-19 disease in some patients.

In yet another embodiment IL31 is a preferred biomarker, since it showed outstanding performance. It activates STAT3 and possibly STAT1 and STAT5 through the IL31 heterodimeric receptor composed of IL31 RA and OSMR (PubMed:15184896). It may function in skin immunity and is reported to enhance myeloid progenitor cell survival in vitro (by similarity).

Thus, without being bound to theory, although in early phase CD28 seems to be upregulated, the downregulation of TNR8 and OX2G, and the upregulation of CXCR5 in later phases of the disease may result in a shift to an overproduction of B-cells, whereas the differentiation and proliferation of T-cells is downregulated. Furthermore, CD28, and TLR3 upregulation may trigger a CRS-reaction (Covid-19 induced cytokine release syndrome). Last, but not least, FGF2 upregulation may influence the whole process in a number of different MAPK signaling cascades.

### Overview of Potential Use of Biomarker Groups for Certain Purposes

The biomarkers of the present invention are all useful for determining the predisposition for a severe or critical course of a COVID-19-disease from a mild or moderate course of a COVID-19-disease in a subject, since they are all significant de-regulated with a |log FC| of at least 0.5, more preferred a |log FC| of at least 1.0, even more preferred a |log FC| of at least 1.5, even more preferred a |log FC| of at least 2.0, and a |log FC| up to 2.5, up to 3.0, or even a |log FC| up to 3.5, up to 4.0, up to 5.0. Thus, biomarkers of a log FC between 0.5 and 5.0 are preferred biomarkers, even more preferred a log FC between 1.0 and 4.0, yet even more preferred a log FC between 1.5 and 3.5, most preferred a log FC between 2.0 and 3.5.

However, for certain methods a utilization of one of the groups listed above may be preferred.

For guidance of the skilled person such a preference is listed in the following table:

TABLE 1
Use of biomarker groups for certain purposes

| | Diagnostic | Predictive | Therapeutic | Analytic | | Other applications/ |
|---|---|---|---|---|---|---|
| Group | Method | Method | Method | Method | Kit | Comments |
| quality score | + | + | + | + | + | preferred as a versatile |
| | | | | | | collection of biomarkers |
| | | | | | | for all applications |
| acute | + | + | | | + | preferred for tests in the |
| | | | | | | acute time-phase |
| acute and medium | + | + | | | + | preferred for tests in the |
| | | | | | | acute and medium time- |
| | | | | | | phase |
| acute, medium | + | + | + | + | + | preferred as a versatile |
| and late | | | | | | collection of biomarkers |
| | | | | | | for all applications |
| medium | | | + | + | + | preferred for tests in the |
| | | | | | | medium time-phase |
| medium and late | | | + | + | + | preferred for tests in the |
| | | | | | | medium and late time-phase |
| late | | | + | + | + | preferred for tests in the |
| | | | | | | late time-phase |
| immune cell | + | + | + | + | + | preferred for tests focusing |
| activation | | | | | | on immune cell activation |
| cytokine production | + | + | + | + | + | preferred for tests with |
| and signaling | | | | | | respect to cytokine production, |
| | | | | | | e.g. the Covid-19 induced |
| | | | | | | cytokine release syndrome |
| RNA-metabolism | + | + | + | + | + | preferred for tests with |
| | | | | | | respect to RNA-metabilism, |
| | | | | | | e.g. viral related RNA |
| | | | | | | metabolism, such as viral |
| | | | | | | replication |
| NF-kappa B | + | + | + | + | + | preferred for tests with |
| | | | | | | respect to general reactions |
| | | | | | | of the immune system during |
| | | | | | | COVID-19 |
| MAPK/ERK | + | + | + | + | + | preferred for tests with |
| signaling | | | | | | respect to cytokine production, |
| | | | | | | e.g. the Covid-19 induced |
| | | | | | | cytokine release syndrome |
| CD4 | + | + | + | + | + | preferred for tests with |
| | | | | | | respect to T cell activation |
| CD47 | + | + | + | + | + | preferred for tests with |
| | | | | | | respect to myeloid cell |
| | | | | | | activation |
| CXCR5 | + | + | + | + | + | preferred for tests with |
| | | | | | | respect to myeloid cell |
| | | | | | | activation and leukocyte |
| | | | | | | chemotaxis |
| ERBB2 | + | + | + | + | + | preferred for tests with |
| | | | | | | respect to cell migration |
| | | | | | | and stress induced MAPK |
| | | | | | | cascade activation |
| best of each | + | + | + | + | + | preferred for applications, |
| cluster | | | | | | which utilize only few |
| | | | | | | biomarkers, e.g. quick-tests |
| best of each | + | + | + | + | + | preferred for applications, |
| cluster and | | | | | | which utilize only few |
| sub-cluster | | | | | | biomarkers, e.g. quick-tests |
| best of | + | + | + | + | + | preferred for applications, |
| cluster 1 | | | | | | which utilize only few |
| | | | | | | biomarkers, e.g. quick-tests |
| best of | + | + | + | + | + | preferred for applications, |
| cluster 2 | | | | | | which utilize only few |
| | | | | | | biomarkers, e.g. quick-tests |
| best of sub- | + | + | + | + | + | preferred for applications, |
| cluster 2d | | | | | | which utilize only few |
| | | | | | | biomarkers, e.g. quick-tests |
| FGF2 | + | + | + | + | + | |
| TNR8 | + | + | + | + | + | |
| CD28 | + | + | + | + | + | |
| OX2G | + | + | + | + | + | |
| CXCR5 | + | + | + | + | + | |
| TLR3 | + | + | + | + | + | |
| IL31 | + | + | + | + | + | |

### Diagnostic Method

The method of the present invention may be used in one embodiment to determine early (early diagnosis) the infection with SARS-CoV-2 in a subject.

As such, this method may be used either alone, or in combination with other diagnostic methods, such as PCR-test, antibody-test and/or symptomatic determination.

The inventive method may be used to integrate one or more of the inventive biomarkers as part of a quick-test or testing device in order to identify the early onset of a COVID-19-disease.

The term ‘early diagnosis’ as used herein refers to a timely diagnosis of a SARS-CoV-2-infection. More preferably, the SARS-CoV-2 infection should be diagnosed within three days before the onset of first symptoms until five days after the onset of first symptoms.

In one embodiment the biomarkers of the “quality score group”, “acute group”, “acute and medium group” and/or “acute, medium and late group” are preferred (see also table 1 for additional suitable groups).

### Predictive Method

The method of the present invention may be used in one embodiment to predict the predisposition for a severe or critical course of a COVID-19-disease in a subject.

The term “predict the predisposition for a severe or critical course of a COVID-19-disease in a subject” may include assessing the probability prior to a COVID-19-related medical intervention and/or therapy in a subject.

More preferably, the risk/probability of occurrence of a severe or critical course of a COVID-19-disease in a subject within up to two weeks after the onset of first symptoms and/or a positive test for a SARS-CoV-2-infection is predicted (“predictive window”). In a preferred embodiment, the predictive window is an interval of at least 1 day, at least 2 days, at least 3 days, at least 4 days, at least 5 days, at least 6 days, at least 1 week, at least 10 days, or at least 2 weeks, or any intermittent time range. In a particular preferred embodiment of the present invention, the predictive window, preferably, is an interval of up to 10 days, or more preferably, of up to 2 weeks. Preferably, said predictive window is calculated from a positive test for a SARS-CoV-2-infection and/or from the onset of first symptoms. Alternatively, said predictive window is calculated from the time point at which the sample to be tested has been obtained.

In one embodiment samples from the subject are taken when said subject was exposed to an elevated risk for a SARS-CoV-2 infection. Such an elevated risk may be for example if said subject had contact of more than 15 minutes with a second subject which was tested positive for a SARS-CoV-2-infection; or said subject stayed for a longer period of time in an area with an elevated risk for a SARS-CoV-2 infection, such as a region, state, or country with more than 50 SARS-CoV-2-infections per 100.000 inhabitants, or said subject works in a working environment with elevated infection risks, such as cold storage houses, secluded rooms with no ventilation, marketplaces, etc.

In another embodiment samples from the subject are taken when said subject belongs to a potential risk group, such as an overweight individual (i.e. with a body mass index (BMI) of more than 30), with an age of >50 years, smoker, with a pre-existing condition selected from the group consisting of cardiovascular disease, chronic lung-disease (e.g. COPD, asthma), diabetes mellitus, chronic liver-disease, cancer, and immunocompromised subjects.

In yet another embodiment samples from the subject are taken when said subject was tested positive for a SARS-CoV-2 infection. The SARS-CoV-2 test is preferentially a standardized PCR-test, and/or a serum-test (antibody test). In another embodiment tests analyzing the T-cell-response may be used for diagnosis. Preferably either in parallel with the SARS-CoV-2 test or within less than 72 hours, less than 48 hours, less than 24 hours, or less than 12 hours after the SARS-CoV-2 test was positive.

The samples are then tested for at least one biomarker which is significantly up- or downregulated in a CS-patients and which therefore indicates the predisposition for a severe or critical course of a COVID-19-disease in said subject.

In one embodiment the biomarkers of the “quality score group”, “acute group”, “acute and medium group” and/or “acute, medium and late group” are preferred (see also table 1 for additional suitable groups).

To increase the significance of the prediction the biomarkers of the groups mentioned above can be combined, that is two or more of said biomarkers are measured and only if all of them show a significant up- or downregulation a prediction is made.

As will be understood by those skilled in the art, such a prediction cannot be correct for 100% of the subjects. The term, however, requires that prediction can be made for a statistically significant portion of subjects in a proper and correct manner. Whether a portion is statistically significant can be determined by the person skilled in the art using various well-known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98%, or at least 99%. The p-values are, preferably, less than 0.1, less than 0.05, less than 0.01, less than 0.005, or less than 0.0001. Preferably, the probability envisaged by the present invention allows that the prediction of an upregulated, normal or downregulated risk will be correct for at least 60%, at least 70%, at least 80%, or at least 90% of the subjects of a given cohort or population. The term, preferably, relates to predicting whether a subject is at elevated risk or reduced risk as compared to the average risk for the occurrence of a severe or critical course of a COVID-19-disease in a subject.

### Therapeutic Method

In one embodiment the biomarkers of the invention may be used to determine before a medical intervention whether a subject may undergo medical intervention, and/or whether certain measures have to be taken prior or during the intervention to reduce the risk of a severe or critical course of a COVID-19-disease. For that purpose, the sample may be taken up to 21 days prior to a planned medical intervention.

A sample obtained prior to medical intervention is, preferably, obtained directly prior to the intervention. It is also contemplated to obtain a sample not more than 9 days, not more than 8 days, not more than 7 days, not more than 6 days, or not more than five days prior to medical intervention or at any time point prior medical intervention, such as within 2h, 6h, 12h, 24h, 36h, 48h, within 7 days before a medical intervention.

In some embodiments the sample may also be taken during or after the medical intervention. A sample obtained after medical intervention preferably, may be obtained within 2h, 6h, 12h, 24h, 36h, 48h or within 7 days after completion of medical intervention.

The biomarkers of the present invention can be used to select the best medical intervention and/or treatment based on the biomarker profile of said patient.

For example, a subject with an upregulated risk for the occurrence of a severe or critical course of a COVID-19-disease may receive earlier therapeutic intervention, for example earlier passive ventilation (i.e. even already at a stage of the disease, e.g. when the blood oxygen level is still >93%), and/or earlier medication with CRS-inhibiting therapeutic agents, such as for example Dexamethansone.

In another embodiment the subject may be “stratified” to receive a specific COVID-19 treatment. The term “stratified” or “stratification” as used herein, means identifying subgroups of patients with distinct mechanisms of disease, or particular responses to treatments. Stratified medicine allows to identify and develop treatments that are effective for particular groups of patients. Ultimately, stratified medicine will ensure that the right patient gets the right treatment at the right time. The biomarkers identified in this application can be used for such a stratified medicine approach.

Understanding the mechanisms underpinning disease is of vital importance in achieving the MRC's ambition of leading science for better health. With the knowledge of the structural, functional, and chemical de-regulations of the biomarkers of the present invention that occur inside cells and tissues improves of CS-COVID-19-patients, it becomes clear that diseases are essentially sub-groups of these various abnormalities. A stratified approach helps develop a deeper mechanistic understanding of these sub-groups, which can lead to the identification of novel targets and treatment strategies.

In one embodiment the therapeutic agent is selected from antiviral drugs including Indinavir, Saquinavir, Lopinavir, Ritonavir, interferon-beta, Remdesivir, Favipiravir, Oseltamivir, Chloroquin, Hydroxychloroquin, Umifenovir; serine proteases, including TMPRSS2 or Camostatmesilat; antibiotics, including Linezolid, Azithromycin, Nemonoxacin or Fluorchinolone; interleukin-6-receptor-antagonists, including Tocilizumab or Sarilumab in mono-therapy or combined with Methotrexate; anti-parasite treatments, including Ivermectin; anticoagulants; glucocorticoids such as Dexamethansone, JAK-inhibitors, MEK-inhibitors (as well as other kinase inhibitors), and any combination thereof.

In another embodiment the therapeutic agent is selected from bamlanivimab (LY-CoV555), convalescent plasma, Itolizumab, etesevimab (JS016/LY-CoV016), casirivimab+imdevimab (REGN-COV2), Sotrovimab (VIR-7831), favipiravir, Regkirona, camostat, Plitidepsin, AT-527 (Altea Pharmaceuticals), AZD7442 (AstraZeneca), AZD1061 (AstraZeneca), AZD8895 (AstraZeneca), MP0420 (molecular partners), ATR-002 (Atriva Therapeutics), XVR011 (ExeVir Bio), COR-101 (Corat Therapeutics), Vilobelimab (Inflarx), IFX-2 (Inflarx), ISA106 (ISA Pharmaceuticals), Aviptadil, Remdesivir (GS-5734), anakinra, Olumatlizumab, baricitinib, apremilast, mCBM40, valsartan, omeprazole, nintedanib, methylprednisolone, linagliptin (Tradjenta), lenalidomide (Revlimid), hyrocortisone, cyclosporine, atorvastatin, artemisin, tavalisse, symbicort, RecAP, pulmicort and/or prednison, and any combination thereof.

In one embodiment the biomarkers of the “acute, medium and late group” are preferred, the biomarkers of the “quality score group” are even more preferred. In yet other embodiments, especially during later phases of the disease, the biomarkers of the “median group”, “median and late group”, and/or “late group” are preferred (see also table 1 for additional suitable groups).

In yet another embodiment biomarkers showing a larger area under the curve (AUC) from the receiver operating characteristic (ROC) curve (ROC AUC-value) are preferred.

Although the present invention pertains primarily for the prediction and diagnosis of COVID-19, the biomarkers disclosed herein may also be used as diagnostic biomarkers in order to identify high-risk patients for developing long-covid after the COVID-19 disease was overcome.

Although the present invention pertains primarily for the prediction and diagnosis of COVID-19, the biomarkers disclosed herein may also be used as diagnostic biomarkers in order to identify high-risk patients in case of other respiratory diseases where the derangement of the immunesystem may play a role, such as for example common cold, influenza, sinusitis, tonsillitis, otitis media, pharyngitis, laryngitis, bacterial pneumonia, pulmonary embolism, tuberculosis, acute asthma, chronic obstructive pulmonary disease, acute respiratory distress syndrome, chronic bronchitis, bronchiectasis, chronic obstructive pulmonary disease (COPD), cystic fibrosis, infection with MRSA, Streptococcus pneumoniae, Staphylococcus aureus, tuberculosis, lung cancer, pneumocystis pneumonia, SARS induced by different coronaviridae such as including SARS-CoV-1, HCoV NL63, HCoV HKU1, MERS-CoV, as well as other viral infections such as with RSV, influenca viridae, etc.

### Analytic Method

In one embodiment the biomarkers of the invention may be used to determine the effectiveness of a medical intervention and/or treatment based on the biomarker profile of said patient.

In such an embodiment blood samples are taken on a regular basis during the COVID-19 treatment and the biomarker profile is monitored over time. In one embodiment a blood sample is taken every 24 hours, in more severe cases every 12, every 6, every 3 hours. In certain embodiments also longer intervals are applicable, e.g. every second day, twice per week, once per week, once per month, and so on.

Improvement of the biomarker-profile of the CS-patient, i.e. a biomarker-profile which is closer to the profile of the biomarker profile of a MM-patient is desired and the medical intervention and/or treatment is adapted accordingly, e.g. by increasing or decreasing the dosage of a therapeutic agent, by changing the therapeutic agent, by starting or stopping a medical intervention, and the like.

In this specific case the different sub-groups identified in this application are of special interest.

In one embodiment a very high cytokine profile in a patient is treated and, thus, biomarkers of the “positive regulation of cytokine production group” are of specific interest for monitoring. The skilled person may treat the patient with cytokine-inhibitors in order to normalize the biomarkers in that group, thereby improving the overall status of the patient.

In another embodiment the MAPK-pathway is associated with virus encapsulation. Thus, a potential antiviral treatment is with inhibitors of this pathway. In that case the biomarkers of the “positive regulation of MAPK cascade group” are of specific interest for monitoring, in one embodiment in combination with the “positive regulation of cytokine production group” In yet another embodiment, since it is known that IGF activity maintains human lung homeostasis and is implicated in pulmonary diseases such as cancer, ARDS, COPD, asthma and fibrosis, a treatment which improves the IGF-activity may be beneficial. In such a case the monitoring of the biomarker IGF1R (quality score 9), and associated proteins, is of specific interest for monitoring.

In another embodiment the analyzing method may also be used to monitor the effectiveness of medical intervention and/or treatments in a medical study program, such as novel drugs, novel vaccines, novel ventilation systems, and the like. A shift of biomarkers towards the profile of a CS-patient is then to be considered a failure of said novel drug, a shift towards the profile of a MM-patient is then to be considered a success. In such studies the biomarkers of the “positive and negative regulation of the immune system/response”—groups may be of specific interest.

In yet another embodiment the analyzing method may also be used to monitor the recovery of a patient after a COVID-19 disease which becomes visible in the normalization of one or more of the respective biomarkers.

Since the biomarkers may change before visible symptoms occur in the patient, the inventive method allows the very early, almost predictive, adjustment and/or change of the mentioned medical intervention and/or treatment before visible and/or tangible symptoms occur in a patient.

### Kit

The present invention also relates to a kit comprising a detection agent for determining the amount of at least one biomarker of this invention, or variants or fragments thereof, and evaluation instructions for establishing differentiation of the predisposition for a severe or critical course of a COVID-19-disease from a mild or moderate course of a COVID-19-disease in a subject.

The biomarker may be selected from any group of biomarkers disclosed herein, as well as combinations thereof and fragments and variants thereof.

In an embodiment, the biomarker is selected from the group consisting of FGF2, CD28, TGFB2, 113R1, IL15, IGKC, I13R2, CSF1, IGF1R, BCAM, CD166, OX2G, CD45RA, TNR16, CXCR5, CCL19, LEUK, ICAM1, TNFL4, GLPB, IL2, PD1 L1, CCL27, IL3, HMGB1, ALBU, SLAF1, CD47, TNFA, TLR3, TBB3, S10A8/9, IL15, TNF11, TFR1, TNR8, CEAM1/3/5/6/8, AREG, HLA-1, CD81, VEGFA, CCL8, IL31, K1C18, IL12B, ERBB2, DPP4, CD45RB, CD8A, IL1A, HAVR2, IGLC1, TNFL6, CCL2, TNR5, ADIPO, ICAM1, CD4, CD38, and IL20, as well as combinations thereof and/or isoforms, fragments and/or variants thereof.

In one embodiment the following biomarkers are preferred: FGF2, CD28, 113R2, CXCR5, ALBU, SLAF1, S10A8/9, CEAM1/3/5/6/8, AREG, CD81, ERBB2, CD8A and/or CCL2.

The term “kit” as used herein refers to a collection of the aforementioned agent and the instructions provided in a ready-to-use manner for determining the biomarker amount in a sample. The agent and the instructions are, preferably, provided in a single container. Preferably, the kit also comprises further components which are necessary for carrying out the determination of the amount of the biomarker. Such components may be auxiliary agents which are required for the detection of the biomarker or calibration standards. Moreover, the kit may, preferably, comprise agents for the detection of more than one biomarker.

Multiple groups have been described above that the biomarker of the present invention may preferably be selected from. These groups have in particular been described with respect to the method of the present invention. However, it is to be understood that these groups are not only relevant with respect to the methods of the invention but also with respect to the kits, devices, and uses of the present invention.

It is particularly envisaged that the detection agents, preferably, an antibody or fragment thereof, comprised by the aforementioned kits or compositions are immobilized on a solid support in an array format. In particular, the detection agents may be immobilized on a solid support and arranged in an array format, e.g., in a so-called “microarray”. Accordingly, the present invention also envisaged a microarray and/or a fluidic device, such as a collateral flow device, comprising the aforementioned detection agents.

Preferably, the kit, the composition and the microarray are used for differentiation of the predisposition for a severe or critical course of a COVID-19-disease from a mild or moderate course of a COVID-19-disease in a subject.

All references cited in this specification are herewith incorporated by reference with respect to their entire disclosure content and the disclosure content specifically mentioned in this specification.

### Further Definitions

The term “positive test for a SARS-CoV-2-infection” as used herein refers to any test which allows the determination of a SARS-CoV-2-infection in a patient. A preferred test may be done by polymerase chain reaction (PCR). In one embodiment realtime-PCT (RT-PCR) is used for testing according to the publication of Corman et al., (Detection of 2019 novel coronavirus (2019-nCoV) by real-time RT-PCR, Eurosurveillance, Volume 25, Issue 3, 23 Jan. 2020). However also other PCR-tests as well as other testings, such as for example immuno-assays (antibody-based tests) may be used.

The term “subject” as used herein relates to animals, preferably mammals, and, more preferably, humans. In an embodiment, the subjects are male or female subjects. The subject to be tested may undergo or may have undergone medical intervention. Preferably, the method is applied to a subject known to undergo COVID-19 treatment.

For example, the subject may have undergone COVID-19 treatment 48 h, 24 h or less before the sample is obtained. In another embodiment, the sample is taken from a subject who will undergo COVID-19 treatment, e.g. within 48, or 24 hours.

### Embodiments

In one embodiment the invention pertains to a method for determining the predisposition for a severe or critical course of a COVID-19-disease from a mild or moderate course of a COVID-19-disease in a subject, comprising the steps of:
a. determining in a sample obtained from the subject the amount of at least one biomarker;
b. determining the difference of the amount of said at least one biomarker to a reference amount for said at least one biomarker;
wherein the sample is obtained at a specific time-phase of the COVID-19 disease;
wherein the reference amount is the amount of the respective biomarker in a subject who has a mild or moderate COVID-19-disease; and
wherein the difference |log FC| is at least 0.5, and

In one embodiment the de-regulation of the biomarker is significant, i.e. has an adjusted p-value of less than 5*10⁻², preferably less than 5*10⁻³, preferably less than 5*10⁻⁴, preferably less than 5*10⁻⁵, preferably less than 5*10⁻⁶, preferably less than 5*10⁻¹, preferably less than 5*10⁻⁸, preferably less than 5*10⁻¹.

In another embodiment the invention pertains to said method, wherein the difference of |log FC| of at least 0.5, preferably at least 1.0, preferably at least 1.5, preferably at least 2.0, preferably at least 2.5, preferably at least 3.0, as compared to the reference amount indicates that the subject has a pre-disposition for a severe or critical course of a COVID-19-disease.

In another embodiment the invention pertains to said method, wherein the quality score of the at least one biomarker in the sample from the subject has a quality score of at least 4, more preferably at least 5, even more preferably at least 6, most preferred of at least 8.

In another embodiment the invention pertains to said method, wherein the ROC AUC-value is at least 0.70, preferably at least 0.75, more preferably at least 0.80, most preferably at least 0.825.

In another embodiment the invention pertains to said method, wherein in the sample from the subject the at least one biomarker is selected from the group consisting of CEAM1/3/5/6/8, SLAF1, S10A8/9, BTLA, AREG, FGF2, CD47, CXCR5, TNR16, 113R2, IGF1R, CD81, CD28, VEGF165b/VEGFA, IL2, IL15, HMGB1, and ALBU, as well as combinations thereof and/or isoforms, fragments and/or variants thereof.

In another embodiment the invention pertains to said method, wherein in the sample from the subject the at least one biomarker is selected from the group consisting of SLAF1, S10A8/9, BTLA, AREG, FGF2, CD47, CXCR5, TNR16, 113R2, IGF1R, CD81, CD28, VEGF165b/VEGFA, IL2, IL15, HMGB1, and ALBU, as well as combinations thereof and/or isoforms, fragments and/or variants thereof.

In another embodiment the invention pertains to said method, wherein in the sample from the subject the at least one biomarker is selected from the group consisting of SLAF1, S10A8/9, BTLA, AREG, FGF2, CD47, CXCR5, 113R2, IGF1R, CD81, VEGFA (especially the isoform VEGF165b), I15, and HMGB1, as well as combinations thereof and/or isoforms, fragments and/or variants thereof.

In another embodiment the invention pertains to said method, wherein in the sample from the subject the at least one biomarker is selected from the group consisting of MUC1, CCL3, CALB1, ANGP2, CATB, ACVL1, MPIP2, SFRP5, IGF1, MTOR, FAF1, SLIP, PRTN3, TYRO3, CADH5, S10AC, SPIT1, S100B, CADH1, LEG4, DMB, RARR2, 2A5D, 122R2, FGF9, CDN1A, ISK, MMP9, SPRC, PEPC, ANGL3, IBP1, IL26, BASI, CORIN, IF127, FABPI, IBP2, AMBP, IL1B, TSP1, S10AD and TOP1, as well as combinations thereof and/or isoforms, fragments and/or variants thereof.

In another embodiment the invention pertains to said method, wherein in the sample from the subject the at least one biomarker is selected from the group consisting of MTOR, UTER, CD14, CADH1, CCL3, FABPI, 122R2, IL26, SPIT1, ANGP2, CATB, SFRP5, BASI, S10AD, and/or SLIP.

In another embodiment the invention pertains to said method, wherein in the sample from the subject the at least one biomarker is selected from the group consisting of a set of at least two biomarkers selected from the group consisting of: AREG, CD81 and FGF2; CD81, HAVR2 and TBB3; CXCR5, CD81 and FGF2; SLAF1 and CXCR5; CCL2 and I13R2; CCL2 and CD81; CCL2 and FGF2; CCL2 and CD47; UTER and I13R2; S10A8/9, CCL2, AREG and ERBB2; S10A8/9, CCL2 and AREG; S10A8/9, CEAM1,3,5,6,8 and ERBB2; S10A8/9 and CD45RB; S10A8/9 and CEAM1,3,5,6,8; S10A8/9, CCL2 and IL15; S10A8/9, CCL2 and VEGF165b; S10A8/9, ALBU and CCL2; S10A8/9, ALBU and I13R2; S10A8/9, ALBU and IGF1R; S10A8/9, CD8A and CCL2; S10A8/9 and ERBB2; TSP1, AREG and SLAF1; CCL2, IGLC1 and I13R2; CD81, SLAF1 and CD14; CD81, SLAF1 and TSP1; CD81 and CD14; CD81, CD14 and CXCR5, as well as combinations thereof and/or isoforms, fragments and/or variants thereof.

In another embodiment the invention pertains to said method, wherein in the sample from the subject the at least one biomarker is selected from the group consisting of a set of at least two biomarkers selected from the group consisting of: AREG, CD81 and FGF2; and/or CD81, HAVR2 and TBB3.

In another embodiment the invention pertains to said method, wherein in the sample from the subject the at least one biomarker is selected from the group consisting of TNR8, CCL8, TNF11, ICAM1, TBB3, AREG, TNFL4, IL3, CD45RB, CEAM1/3/5/6/8, SLAF1, TNFA, CCL19, IGKC, HMGB1, CCL27, CD47, CD45RA, GLPB, LEUK, PD1 L1, TNR16, IL2, TLR3, CXCR5, OX2G, I13R2, BCAM, IGF1R, CSF1, TGFB2, FGF2, CD166, IL15, CD28, and I13R1, as well as combinations thereof and/or isoforms, fragments and/or variants thereof.

In another embodiment the invention pertains to said method, wherein in the sample from the subject the at least one biomarker is selected from the group consisting of CCL19, IGKC, HMGB1, CCL27, CD47, CD45RA, GLPB, LEUK, PD1 L1, TNR16, IL2, TLR3, CXCR5, OX2G, I13R2, BCAM, IGF1R, CSF1, TGFB2, FGF2, CD166, IL15, CD28, and I13R1, as well as combinations thereof and/or isoforms, fragments and/or variants thereof.

In another embodiment the invention pertains to said method, wherein in the sample from the subject the at least one biomarker is selected from the group consisting of PD1 L1, TNR16, IL2, TLR3, CXCR5, OX2G, I13R2, BCAM, IGF1R, CSF1, TGFB2, FGF2, CD166, IL15, CD28, and I13R1, as well as combinations thereof and/or isoforms, fragments and/or variants thereof.

In another embodiment the invention pertains to said method, wherein in the sample from the subject the at least one biomarker is selected from the group consisting of IL15, 113R1, FGF2, CD28, and CD166, as well as combinations thereof and/or isoforms, fragments and/or variants thereof.

In another embodiment the method according to the invention comprises at least two biomarkers, wherein the at least two biomarkers are selected from at least two different cluster-groups selected from cluster 1-group, cluster 2-group and cluster 3-group.

In another embodiment the invention pertains to said method, wherein the at least two biomarkers selected from at least two different cluster-groups have the highest quality score within this group.

In another embodiment the invention pertains to said method, wherein the at least two biomarkers are selected from CD28, 113R1, IL3, TNF11, CD166, CCL19, OX2G, TNR16, and HMBG1.

In another embodiment the invention pertains to said method, wherein a group of three biomarkers are selected from IL3, OX2G, and CD28; or IL3, OX2G and TNR16; or IL3, CD28, and CCL19; or IL3, CD28, and CD166; or IL3, TNF11, and I13R1; or IL3, TNF11, and TNR16.

In another embodiment the invention pertains to said method, wherein the sample is a urine, blood, plasma or serum sample.

In another embodiment the invention pertains to said method, wherein the sample is taken prior to a planned COVID-19 medical intervention and/or therapy such as administration of a drug, avoiding administration of a drug, artificial respiration treatment, extracorporeal membrane oxygenation (ECMO) or a surgical intervention.

In another embodiment the invention pertains to said method, wherein the time-phase when the sample is taken at “acute time phase”, i.e. the time-phase is between less than 9 days, 5 days, 48 hours, 36 hours, or 24 hours after the subject has been tested positive for a SARS-CoV-2-infection and up to 1, up to 2, up to 3 days before the subject has been tested positive for a SARS-CoV-2-infection and/or from the onset of first symptoms.

In another embodiment the invention pertains to said method, wherein the time-phase when the sample is taken is less than 9 days after the subject has been tested positive for a SARS-CoV2-infection and/or from the onset of first symptoms.

In another embodiment the invention pertains to said method during “acute time phase”, including determining in the sample the amount is in-creased for at least one biomarker selected from the group consisting of group consisting of TNR5, TNF11, CD38, CD4, IL1A, TFR1, TNFL6, CD8A, IL20, CCL2, IL12B, VEGFA, HLA-1, ICAM1, CD81, ERBB2, S10A8/9, HMGB1, 113R2, CCL27, K1C18, CD47, TBB3, AREG, CD45RA, TNFL4, IL3, CD45RB, CEAM1/3/5/6/8, GLPB, I13R1, PD1 L1, LEUK, BCAM, TNR16, SLAF1, FGF2, CD166, TNFA, IGF1R, IL2, CSF1, TLR3, IL15, TGFB2, CXCR5, and CD28, as well as combinations thereof and/or isoforms, fragments and/or variants thereof.

In one embodiment the following biomarkers are preferred: CD8A, CCL2, CD81, ERBB2, S10A8/9, 113R2, AREG, CEAM1/3/5/6/8, TNR16, SLAF1, FGF2, CXCR5 and/or CD28, and may be further combined with S10A8/9.

In another embodiment the invention pertains to said method during “acute time phase”, including determining in the sample the amount is downregulated for at least one biomarker selected from the group consisting of TNR8, OX2G, CCL19, IGKC, ALBU, CCL8, DPP4, HAVR2, IGLC1, IL31, and ADIPO, as well as combinations thereof and/or isoforms, fragments and/or variants thereof.

In one embodiment the following biomarkers are preferred: ALBU and/or IGLC1.

In another embodiment the invention pertains to said method, wherein the time-phase when the sample is taken at “medium time phase”, i.e. between 9 and 21 days after the subject has been tested positive for a SARS-CoV-2-infection and/or from the onset of first symptoms.

In another embodiment the invention pertains to said method during “medium time phase”, including determining in the sample the amount is in-creased for at least one biomarker selected from the group consisting of TNFL6, IL12B, K1C18, IL3, CD81, CEAM1/3/5/6/8, LEUK, S10A8/9, CD45RA, CD45RB, VEGFA, GLPB, IL2, IL15, IL1A, CSF1, CD4, CD28, CD47, TGFB2, CCL27, TNF11, TBB3, 113R1, CD166, HMGB1, CXCR5, 113R2, PD1 L1, AREG, BCAM, IGF1R, TLR3, TNR16, FGF2, HGF, CD276, CTLA4, INHBA, TR13B, OSTP, BTLA, CD3deg, TNR6, SLAF8, NCAM1, IFNL2, and ANGP4, as well as combinations thereof and/or isoforms, fragments and/or variants thereof.

In one embodiment the following biomarkers are preferred: CD81, CEAM1/3/5/6/8, S10A8/9, CD28, CD47, CXCR5, 113R2, AREG, FGF2, and/or SLAF8.

In another embodiment the invention pertains to said method during “medium time phase”, including determining in the sample the amount is downregulated for at least one biomarker selected from the group consisting of OXG2, CCL8, IL31, CCL19, AMPN, TSLP, CCL7, CXCL13, HLA-II, TNFB, CO3, IFNG, and CCL28, as well as combinations thereof and/or isoforms, fragments and/or variants thereof.

In another embodiment the invention pertains to said method, wherein the time-phase when the sample is taken at “late time phase”, i.e. the sample is taken is more than 21 days after the subject has been tested positive for a SARS-CoV-2-infection and/or from the onset of first symptoms.

In another embodiment the invention pertains to said method during “late time phase”, including determining in the sample the amount is in-creased for at least one biomarker selected from the group consisting of IL2, IL15, IL1A, TNF11, CCL27, CSF1, TGFB2, CD4, CD28, CD47, TBB3, CD166, HMGB1, CXCR5, 113R1, 113R2, PD1 L1, AREG, BCAM, IGF1R, TLR3, TNR16, FGF2, CCL2, ICAM1, TNR5, TFR1, TNR6, NCAM1, CD3deg, IFNL2, ANGP4, BTLA, SLAF8, TR11B, CD15, IL22, IL2RA, FLT3, PLF4, PRIO, LIF, ITAM, CD72, NP1L4, ONCM, ITA2B, and HLA-ABC, as well as combinations thereof and/or isoforms, fragments and/or variants thereof.

In another embodiment the invention pertains to said method during “late time phase”, including determining in the sample the amount is downregulated for at least one biomarker selected from the group consisting of TNR8, IGKC, OX2G, CCL8, IL31, CCL19, HLA-II, CXCL13, AMPN, TSLP, CCL7, EGLN, AGRE5, CXCL9, SCRB2, SLAF1, and CCL3, as well as combinations thereof and/or isoforms, fragments and/or variants thereof.

In yet another embodiment the invention pertains method, wherein one, two, three or more biomarkers are determined to be elevated in the sample by a log FC of more than 0.61, wherein the one, two, three or more biomarkers are selected from the group consisting of FGF2, CD28, TGFB2, 113R1, IL15, 113R2, CSF1, IGF1R, BCAM, CD166, CD45RA, TNR16, CXCR5, LEUK, ICAM1, TNFL4, GLPB, IL2, PD1 L1, CCL27, IL3, HMGB1, SLAF1, CD47, TNFA, TLR3, TBB3, S10A8/9, TNF11, TFR1, CEAM1/3/5/6/8, AREG, HLA-1, CD81, VEGFA, K1C18, IL12B, ERBB2, CD45RB, CD8A, IL1A, TNFL6, CCL2, TNR5, CD4, CD38 and IL20.

In yet another embodiment the invention pertains to a method, wherein one, two, three or more biomarkers are determined to be elevated in the sample by a log FC of more than 0.61, wherein the one, two, three or more biomarkers are selected from the group consisting of TNR5, TNF11, CD38, CD4, IL1A, TFR1, TNFL6, CD8A, IL20, CCL2, IL12B, VEGFA, HLA-1, ICAM1, CD81, ERBB2, S10A8/9, HMGB1, 113R2, CCL27, K1C18, CD47, TBB3, AREG, CD45RA, TNFL4, IL3, CD45RB, CEAM1,3,5,6,8, GLPB, I13R1, PD1 L1, LEUK, BCAM, TNR16, SLAF1, FGF2, CD166, TNFA, IGF1R, IL2, CSF1, TLR3, IL15, TGFB2, CXCR5 and CD28, as well as combinations thereof and/or isoforms, fragments and/or variants thereof.

In another embodiment the invention pertains to said method, wherein in the sample from the subject the biomarker is upregulated by a log FC more than 2.50 during acute phase for at least one biomarker selected from the group consisting of CD166, TNFA, IGF1R, IL2, CSF1, TLR3, IL15, TGFB2, CXCR5, and CD28, or isoforms, fragments or variants thereof.

In another embodiment the invention pertains to a method, wherein one, two, three or more biomarkers are determined to be lowered in the sample by a log FC of less than −1.04, wherein the one, two, three or more biomarkers are selected from the group consisting of IGKC, OX2G, CCL19, ALBU, TNR8, CCL8, IL31, DPP4, HAVR2, IGLC1 and ADIPO.

In another embodiment the invention pertains to said method, wherein in the sample from the subject the biomarker is downregulated by a log FC of less than −1.62 during acute phase for at least one biomarker selected from the group consisting of TNR8, OX2G, CCL19, IGKC, ALBU, and CCL8, as well as combinations thereof and/or isoforms, fragments and/or variants thereof.

In another embodiment the invention pertains to a method, wherein one, two, three or more biomarkers are de-regulated over the whole course of the disease during acute, medium and late phase and are selected from the group consisting of CXCR5, CD28, IL15, IGF1R, IL2, TGFB2, TNR16, TLR3, FGF2, CD4, CSF1, 113R1, TBB3, BCAM, CD166, PD1L2, HMGB1, CD47, 113R2, CCL27, AREG, IL1A, OX2G, CCL8, IL31, CCL19, and TNF11, as well as combinations thereof and/or isoforms, fragments and/or variants thereof.

In another embodiment the invention pertains to a method, wherein one, two, three or more biomarkers are de-regulated over the whole course of the disease during acute, medium and late phase, have a quality score of at least 4 and are selected from the group consisting of CXCR5, CD28, IL15, IGF1R, IL2, TGFB2, TNR16, TLR3, FGF2, CSF1, 113R1, TBB3, BCAM, CD166, PD1L1, HMGB1, CD47, 113R2, CCL27, AREG, OX2G, CCL8, IL31, CCL19, and TNF11, as well as combinations thereof and/or isoforms, fragments and/or variants thereof.

In another embodiment the invention pertains to a method, wherein the fragments, isoforms and/or variants of the biomarkers have at least 70%, at least 80%, at least 90%, at least 95%, at least 98%, or at least 99% sequence identity with the biomarker over the whole length of the sequence.

In another embodiment the invention pertains to a method, wherein determining the amount of said at least one biomarker comprises using an immunoassay device, such as ELISA (enzyme-linked immunosorbent assay) or antibody array, in particular a planar antibody microarray or a bead based antibody microarray.

In another embodiment the invention pertains to a method, wherein determining the amount of said at least one biomarker comprises
a. contacting the biomarker with a specific ligand,
b. removing non-bound ligand, and
c. measuring the amount of bound ligand.

In another embodiment the invention pertains to a method, wherein determining the amount of said at least one biomarker comprises
a. contacting a solid support comprising a ligand for the biomarker as specified above with a sample comprising the biomarker,
b. optionally, removing non-bound biomarker, and
c. measuring the amount of biomarker which is bound to the support.

In another embodiment the invention pertains to a method, wherein the response to the COVID-19 treatment is monitored by a normalization of the amount of the biomarkers to the reference amount.

In another embodiment the invention pertains to a method, wherein the sample is taken prior to a planned COVID-19 medical intervention and/or therapy such as administration of a drug, avoiding administration of a drug, artificial respiration treatment, extracorporeal membrane oxygenation (ECMO) or a surgical intervention.

In another embodiment the invention pertains to a method, wherein the sample is a urine, blood, plasma or serum sample.

In another embodiment the invention pertains to a method used as a diagnostic method, a predictive method, a therapeutic method and/or an analytic method.

In another embodiment the invention pertains to a device for identification the predisposition for a severe or critical course of a COVID-19-disease in a subject, comprising
an analyzing unit for the said sample of the subject comprising a detection agent for at least one biomarker, or variants or fragments thereof, said detection agent allowing for the determination of the amount of the said at least one biomarker in the sample, and
an evaluation unit comprising a data processing unit and a data base, said data base comprising a stored reference and said data processing unit being capable of carrying out a comparison of the amount of the at least one biomarker determined by the analyzing unit and the stored reference thereby establishing the prediction,
wherein in the sample from the subject the at least one biomarker is selected from the group consisting of CEAM1/3/5/6/8, SLAF1, S10A8/9, BTLA, AREG, FGF2, CD47, CXCR5, TNR16, 113R2, IGF1R, CD81, CD28, VEGF165b/VEGFA, IL2, IL15, HMGB1, and ALBU, as well as combinations thereof and/or isoforms, fragments and/or variants thereof;
and/or, wherein in the sample from the subject the at least one biomarker is selected from the group consisting of MUC1, CCL3, CALB1, ANGP2, CATB, ACVL1, MPIP2, SFRP5, IGF1, MTOR, FAF1, SLIP, PRTN3, TYRO3, CADH5, S10AC, SPIT1, S100B, CADH1, LEG4, DMB, RARR2, 2A5D, 122R2, FGF9, CDN1A, ISK, MMP9, SPRC, PEPC, ANGL3, IBP1, IL26, BASI, CORIN, IF127, FABPI, IBP2, AMBP, IL1B, TSP1, S10AD and TOP1, as well as combinations thereof and/or isoforms, fragments and/or variants thereof;
and/or, wherein in the sample from the subject the at least one biomarker is selected from the group consisting of a set of at least two biomarkers selected from the group consisting of: AREG, CD81 and FGF2; CD81, HAVR2 and TBB3; CXCR5, CD81 and FGF2; SLAF1 and CXCR5; CXCR5, CD81 and FGF2; CCL2 and I13R2; CCL2 and CD81; CCL2 and FGF2; CCL2 and CF47; UTER and I13R2; S10A8/9, CCL2, AREG and ERBB2; S10A8/9, CCL2 and AREG; S10A8/9, CEAM1,3,5,6,8 and ERBB2; S10A8/9 and CD45RB; S10A8/9 and CEAM1,3,5,6,8; TSP1, AREG and SLAF1; CCL2, IGLC1 and I13R2; CD81, SLAF1 and CD14; CD81, SLAF1 and TSP1; CD81, TSP1 and SLAF1; CD81 and CD14; CD81, CD14 and CXCR5, as well as combinations thereof and/or isoforms, fragments and/or variants thereof;
and/or, wherein the biomarker is at least one selected from the group consisting of I13R1, FGF2, IL15, 113R2, CD166, CD28, TGFB2, CSF1, IGF1R, BCAM, OX2G, CD45RA, TNR16, CXCR5, CCL19, IL2, PD1 L1, CCL27, HMGB1, CD47, TLR3, LEUK, GLPB, and IGKC as well as isoforms, fragments and/or variants thereof. In another embodiment the biomarker is at least one selected from the group consisting of IL3, OX2G, and CD28; or IL3, OX2G and TNR16; or IL3, CD28, and CCL19; or IL3, CD28, and CD166; or IL3, TNF11, and I13R1; or IL3, TNF11, and TNR16, as well as combinations thereof and/or isoforms, fragments and/or variants thereof.

In another embodiment the invention pertains to a kit comprising a detection agent for determining the amount of at least one biomarker, and evaluation instructions for establishing the identification the predisposition for a severe or critical course of a COVID-19-disease in a subject, wherein the biomarker is at least one selected from the group consisting of I13R1, FGF2, IL15, 113R2, CD166, CD28, TGFB2, CSF1, IGF1R, BCAM, OX2G, CD45RA, TNR16, CXCR5, CCL19, IL2, PD1 L1, CCL27, HMGB1, CD47, TLR3, and IGKC as well as isoforms, fragments and/or variants thereof. In another embodiment the biomarker is at least one selected from the group consisting of IL3, OX2G, and CD28; or IL3, OX2G and TNR16; or IL3, CD28, and CCL19; or IL3, CD28, and CD166; or IL3, TNF11, and I13R1; or IL3, TNF11, and TNR16.

In another embodiment the invention pertains to said kit, wherein the detection agent is selected from the group consisting of antibodies and aptamers.

In one embodiment the detection-kit (and or device) may be selected from
1. a standard ELISA adapted for clinical testing
2. lateral flow device with multiplexing options
3. point of care device (POC)
4. bead array
5. antibody array integrated in POC.

In another embodiment the invention pertains to a use of a method, device or kit for identification the predisposition for a severe or critical course of a COVID-19-disease in a subject or for early diagnosis of a COVID-19 disease.

In another embodiment the invention pertains to said use, for monitoring a patient group to be treated with a therapeutic agent which is selected from antiviral drugs including Indinavir, Saquinavir, Lopinavir, Ritonavir, interferon-beta, Remdesivir, Favipiravir, Oseltamivir, Chloroquin, Hydroxychloroquin, Umifenovir; serine proteases, including TMPRSS2 or Camostatmesilat; antibiotics, including Linezolid, Azithromycin, Nemonoxacin or Fluoro-chinolone; interleukin-6-receptor-antagonists, including Tocilizumab or Sarilumab in mono-therapy or combined with Methotrexate; anti-parasite treatments, including Ivermectin; anticoagulants; glucocorticoids such as Dexamethason, JAK-inhibitors, MEK-inhibitors (as well as other kinase inhibitors, i.e. MAPK/ERK-inhibitor), and any combination thereof.

In another embodiment the invention pertains to said use, for monitoring a patient group to be treated with a therapeutic agent which is selected bamlanivimab (LY-CoV555), convalescent plasma, Itolizumab, etesevimab (JS016/LY-CoV016), casirivimab+imdevimab (REGN-COV2), Sotrovimab (VIR-7831), favipiravir, Regkirona, camostat, Plitidepsin, AT-527 (Altea Pharmaceuticals), AZD7442 (AstraZeneca), AZD1061 (AstraZeneca), AZD8895 (AstraZeneca), MP0420 (molecular partners), ATR-002 (Atriva Therapeutics), XVR011 (ExeVir Bio), COR-101 (Corat Therapeutics), Vilobelimab (Inflarx), IFX-2 (Inflarx), ISA106 (ISA Pharmaceuticals), Aviptadil, Remdesivir (GS-5734), anakinra, atlizumab, baricitinib, apremilast, mCBM40, valsartan, omeprazole, nintedanib, methylprednisolone, linagliptin (Tradjenta), lenalidomide (Revlimid), hyrocortisone, cyclosporine, atorvastatin, artemisin, tavalisse, symbicort, RecAP, pulmicort and/or prednison, and any combination thereof.

In some embodiments the biomarkers IL2, TNFB, and VEGFA are less preferred to be included in the biomarker selection. Thus, in some embodiments, the biomarker-group may be selected from the ones as disclosed hereinunder, but without IL2, TNFB, and VEGFA.

The biomarkers IL3, IL12, IL15, CCL2, CCL3, CCL27, CXCL9, IFNL2, TNFA, CSF1 (MCSF), TSLP, and FGF2 could be shown in the present invention to be significant differentiators between CS- and MM-patients, although they were reported as “not significant” in the literature. Thus, despite the disclosure in the prior art, they may be used as differentiating biomarkers in the sense of this invention.

### SHORT DESCRIPTION OF THE FIGURES

FIG. 1 Venn-diagram of the biomarkers which are up- or downregulated during the different time-phases. Acute phase (<=9 days) is the upper left circle, medium phase (10 to 21 days) is the upper right circle, and late phase (>21 days) is the lower circle in the middle. Upregulated biomarkers are depicted by a “+”, downregulated biomarkers are marked by “−”.

FIG. 2 The abundance levels (M-values) of biomarkers with significant differential abundance in CS-patients and MM-patients in acute phase, in medium phase and in late phase were subjected to a sample-wise unsupervised hierarchical clustering. For the clustering the euclidian distance and complete agglomeration was used.

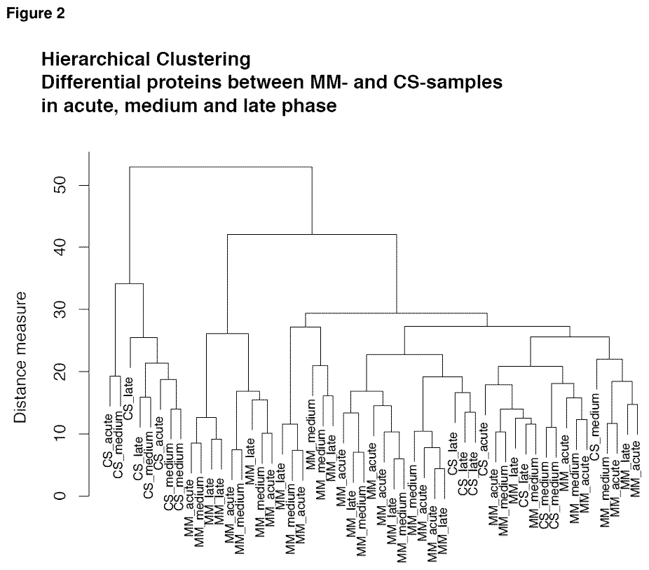

FIG. 3 The volcano plot visualises distinct abundance variations of tested biomarkers between CS samples and MM samples from acute phase. The corresponding log-fold changes (log FC) is plotted for each biomarker on the x-axis, the significance level as p-value adjusted for multiple testing on the y-axis. A significance level of adj. p-value=0.05 is indicated as a horizontal line. The log FC-cutoffs are indicated as vertical lines. Biomarkers with a positive log FC have a higher abundance in acute CS-patients, biomarkers with a negative value in acute MM samples.

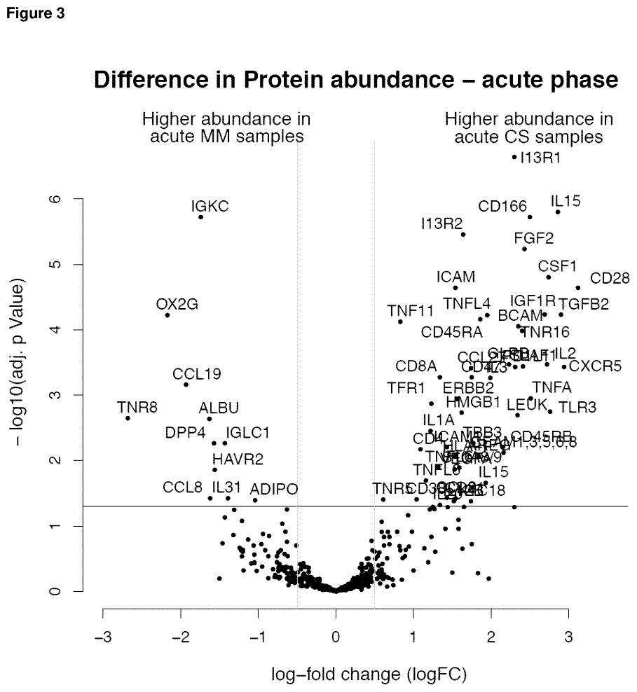

FIG. 4 The boxplot compares the abundance in the study groups for each of the 24 biomarkers with highest quality score. In the plot for each biomarker from left to right are depicted: MM-patients in acute (A), medium (M) and late (L) phase and CS-patients in acute (A), medium (M) and late (L) phase. In addition, the adjusted p-value comparing MM- and CS-patients is indicated. Each box represents the median of the sample group as a line. The box represents the interquartile range (IQR) from first (Q1) to third quartile (Q3). The whiskers depict the minimum (Q1−1.5×IQR) and maximum (Q4−1.5) values. All values beyond this range are depicted as dots indicating an outlier status of the respective samples.

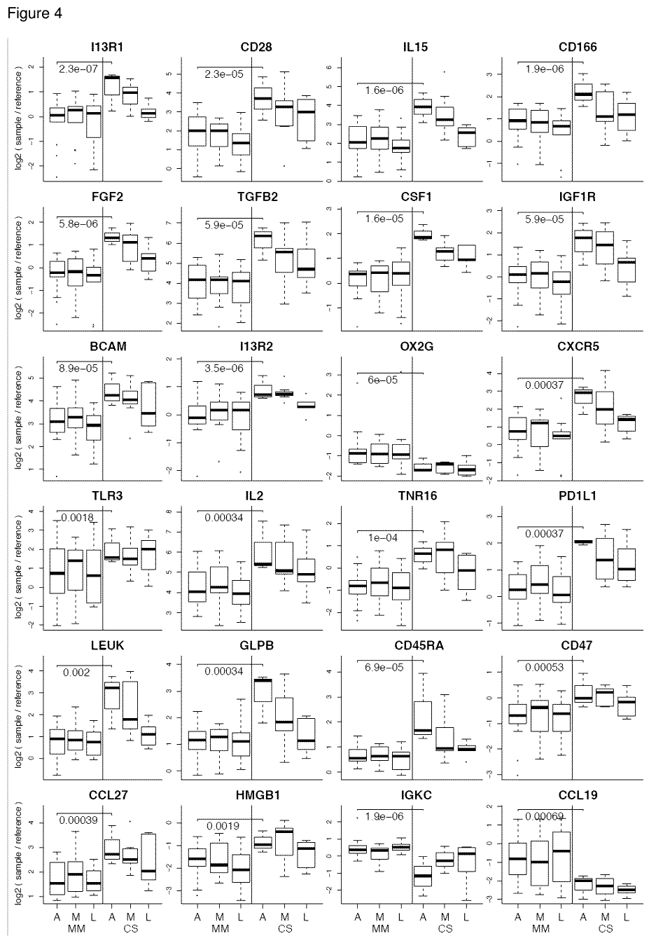

FIG. 5 Protein-protein interactions of biomarkers in acute phase with significant differential abundance in the acute phase of COVID-19 disease are shown. The plot and data were derived from STRING and adapted to show the biomarker protein names instead of gene names. For the string analysis the interaction sources experiments, databases, co-expression, neighbourhood, gene fusion and co-occurrence were used and the confidence cut-off was set to medium level (0.4).

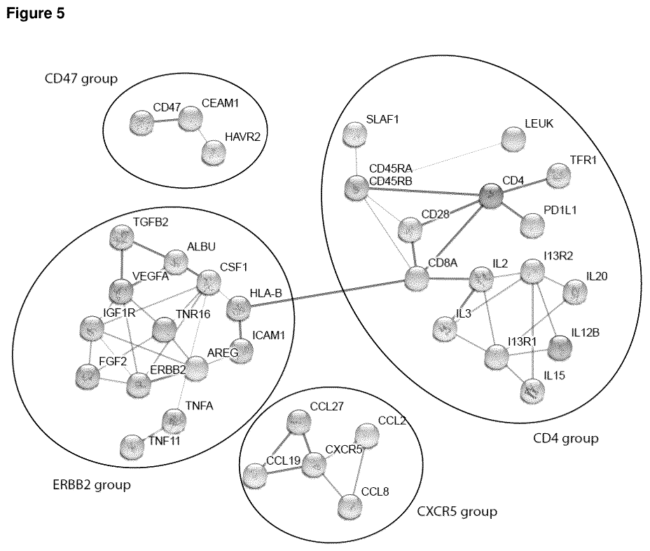

FIG. 6 For proteins differential in acute phase a heatmap was drawn based on the M-values derived from the antibody microarray analysis. M-values are log ratios of the two colour channels for the signal intensity of a biomarker in the sample and signal intensity in the reference sample. In the heatmap each row refers to a biomarker, while the patient samples are shown in the columns. Patient samples as well as proteins are clustered using hierarchical clustering based on euclidian distance and complete linkage agglomeration. The proteins form three main clusters 1, 2 and 3 with similar abundance profiles in the patient population. The first and second cluster can be separated in 4 distinct sub-clusters 1a, 1b, 1c, 1d and 2a, 2b, 2c and 2d.

FIG. 7 For a better illustration the protein clustering from FIG. 6 is presented here excluding the heatmap to depict the grouping of the individual biomarkers in the clusters (1,2, and 3) as well as in the sub-clusters (1a-1d and 2a-2d). Please note that IL15 was recognized by two antibodies, which show a different clustering to cluster 1 and cluster 3. It is believed that the classification of IL15 to cluster 1 is an artefact.

### EXAMPLES

### Example 1

In the studies underlying this invention, plasma samples from subjects infected with SARS-CoV2 as measured by PCR-test were analyzed using antibody microarrays comprising 527 antibodies against 349 different potential biomarkers (mostly proteins, with one exception, which is CD15) with a selection of cellular signaling molecules and cell surface proteins.

It was assessed whether there are differences between subjects with a mild or moderate course of COVID-19 infection (MM-patients) and subjects with a severe or critical course of COVID-19 infection (CS-patients). Differences in the biomarker amounts between subjects that turned out to be statistically significant are those of Table 1 below. These biomarkers may be used as biomarkers for differentiation of the predisposition for a severe or critical course of a COVID-19-disease from a mild or moderate course of a COVID-19-disease in a subject.

In order to identify biomarkers with differential abundance in MM- and CS-patients a study was performed utilizing complex antibody microarrays.

The samples were labelled at an adjusted protein concentration for two hours with scioDye 1. In addition, a reference sample was established by pooling identical volume of each sample and labelling it for two hours with scioDye 2. After two hours the reaction was stopped. All labelled protein samples were stored at −20° C. until use.

The 53 samples were analysed in a dual-colour approach using a reference-based design on 53 scioCD antibody microarrays (Sciomics) targeting different CD surface biomarkers and cytokines/chemokines. Each antibody is represented on the array in four replicates.

The arrays were blocked with scioBlock (Sciomics) on a Hybstation 4800 (Tecan, Austria) and afterwards the samples were incubated competitively with the reference sample using a dual-colour approach. After incubation for three hours, the slides were thoroughly washed with 1×PBSTT, rinsed with 0.1×PBS as well as with water and subsequently dried with nitrogen.

Slide scanning was conducted using a Powerscanner (Tecan, Austria) with identical instrument laser power and constant PMT settings. Spot segmentation was performed with GenePix Pro 6.0 (Molecular Devices, Union City, CA, USA). Acquired raw data were analysed using the linear models for microarray data (LIMMA) package of R-Bioconductor after uploading the medium signal intensities. For normalisation, a specialised invariant Lowess method was applied. For differential analysis of the samples alinear model was fitted with LIMMA resulting in a two-sided t-test or F-test based on moderated statistics. All presented p values were adjusted for multiple testing by controlling the false discovery rate according to Benjamini and Hochberg. Proteins were defined as differential for |log FC|>0.5 and an adjusted p value <0.05.

Differences in protein abundance between different samples or sample groups are presented as log-fold changes (log FC) calculated for the basis 2. In a study comparing samples versus control a log FC=1 means that the sample group had on average a 2¹=2 fold higher signal as the control group. log FC=−1 stands for 2⁻¹=½ of the signal in the sample as compared to the control group.

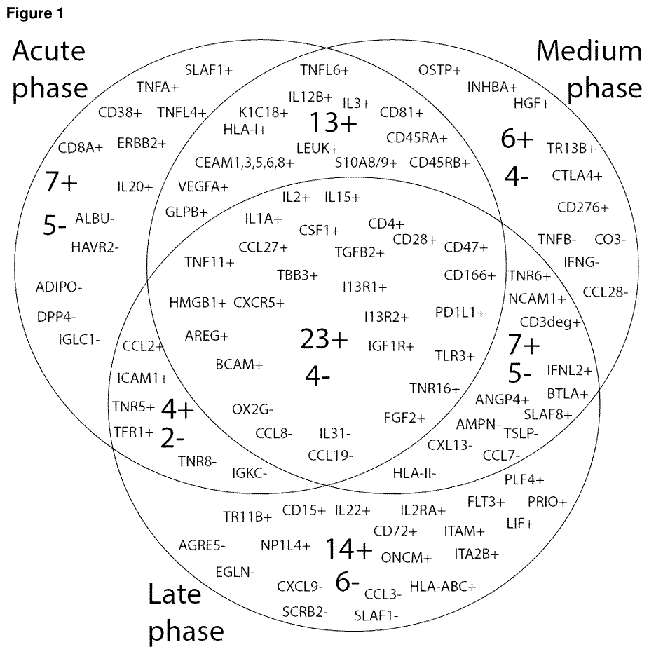

Using LIMMA analysis, 58 proteins were identified with differential abundance between MM- and CS-patients as differential in acute phase (see also FIG. 1, 3 and table 2), as defined above. In addition, 41 proteins and a tetrasaccharide carbohydrate were identified with differential abundance between MM- and CS patients in medium and/or late phase, as defined above (see also tables 3-5).

As outlined before, the biomarkers of the present invention were identified by binding to immobilized antibodies. Some antibodies recognize more than a single protein, thus, in these cases at least one of the recognized biomarkers is significantly up- or downregulated with a log FC above the threshold value. These biomarkers are:
One antibody recognizes both S100A8 (in some embodiments S10A8) as well as S100A9 (in some embodiments S10A9) and their complex. Thus, whenever the short name “S100A8/9” or in some embodiments “S10A8/9”) is used hereinunder, at least one member is meant selected from the group consisting of S100A8 and S100A9, as well as combinations thereof and/or isoforms, fragments and/or variants thereof. The complex S10A8/A9 is also known as Calprotectin.
One antibody recognizes CEAM 1, 3, 5, 6 and 8. Thus, whenever the short name “CEAM1/3/5/6/8” is used hereinunder, at least one member is meant selected from the group consisting of CEAM 1, 3, 5, 6 and 8, as well as combinations thereof and/or isoforms, fragments and/or variants thereof.
One antibody recognizes all isoforms of CD45 which comprise exon A (i.e. ABC, AB and A). Thus, whenever the short name “CD45RA” is used hereinunder, any CD45-variant comprising exon A is meant, as well as combinations thereof and/or isoforms, fragments and/or variants thereof.
One antibody recognizes all isoforms of CD45 which comprise exon B (i.e. ABC, AB, BC and B). Thus, whenever the short name “CD45RB” is used hereinunder, any CD45-variant comprising exon B is meant, as well as combinations thereof and/or isoforms, fragments and/or variants thereof.
One antibody recognizes all proteins belonging to the HLA class I histocompatibility antigen. Thus, whenever the short name “HLA-1” is used hereinunder, any variant of the HLA class I histocompatibility antigen-family is meant, as well as combinations thereof and/or isoforms, fragments and/or variants thereof.
One antibody recognizes all proteins belonging to the HLA class II histocompatibility antigen. Thus, whenever the short name “HLA-II” is used hereinunder, any variant of the HLA class II histocompatibility antigen-family is meant, as well as combinations thereof and/or isoforms, fragments and/or variants thereof.
One antibody recognizes all proteins belonging to the HLA classes A, B and C. Thus, whenever the short name “HLA-ABC” is used hereinunder, any variant of the HLA class A, B, C histocompatibility antigen-family is meant, as well as combinations thereof and/or isoforms, fragments and/or variants thereof.
One antibody recognizes all variants of the CD3-antigen: CD3D, CD3E and CD3G. Thus, whenever the short name “CD3deg” is used hereinunder, any variant of CD3-antigen, namely CD3D, CD3E and CD3G is meant, as well as combinations thereof and/or isoforms, fragments and/or variants thereof.

One antibody recognizes VEGF165b (SEQ ID No. 152) an endogenous isoform of VEGFA (SEQ ID No 45). Thus, whenever the short name “VEGF165b” is used hereinunder, the VEGF165b variant of VEGFA is meant, as well as isoforms, fragments and/or variants thereof. The results of the aforementioned study are summarized in the following tables. In the tables the difference of protein abundance in the two sample groups is given by the log fold change (log FC) calculated for the basis 2. The level of significance is indicated by the p-value adjusted for multiple testing as described above.

“log FC” is defined as the log fold change calculated for the basis 2 and represents the differences in protein abundance between CS-patients and MM-patients. In the case of the predictive method, “CS-patient” refers to a subject who has a severe or critical COVID-19 disease. A log FC=1 means that CS-patients have on average a 21=2 fold higher signal as compared to MM−patients. log FC=−1 stands for 2⁻¹=1/2 of the signal in CS-patients compared to MM-patients.

“adjusted p-values” are p-values adjusted for multiple testing and indicate the level of significance.

To assess the quality of the biomarkers a Quality score (“QS” or “Qual Score”) was calculated for each biomarker taking into account log FC values, adjusted p-values as well as the linearity and coherency of the “plot over time” during acute, intermediate and late phase as described above.

The sum of such points results in a “quality score” (“QS”) as depicted in the example section in table 2.

The present invention comprises in total 99 biomarkers suitable for the present invention. Biomarkers de-regulated during acute phase are shown in table 2. In medium and late phase additional biomarkers could be identified which were above the log FC-threshold and showed significant p-values. These biomarkers are listed in tables 3, 4 and 5.

TABLE 2
Biomarkers differential in acute phase

| Time-point of differentiating measurement: | | | | | | | | | |
|---|---|---|---|---|---|---|---|---|---|
| | Uniprot entry name | | | acute phase | | medium phase | | late phase | |
| SEQ | (short name in | | Uniprot | | adj. p- | | adj. p- | | adj. p- |
| ID No. | brackets) | QS | accession | logFC | Val | logFC | Val | logFC | Val |
| 01 | FGF2_HUMAN | 8 | P09038 | 2.43 | 5.8E−6 | 1.63 | 1.7E−5 | 1.52 | 1.6E−3 |
| | (FGF2) | | | | | | | | |
| 02 | CD28_HUMAN | 8 | P10747 | 3.12 | 2.3E−5 | 1.99 | 1.4E−4 | 2.39 | 2.2E−4 |
| | (CD28) | | | | | | | | |
| 03 | TGFB2_HUMAN | 7 | P61812 | 2.90 | 5.9E−5 | 1.78 | 5.1E−4 | 1.81 | 5.2E−3 |
| | (TGFB2) | | | | | | | | |
| 04 | I13R1_HUMAN | 9 | P78552 | 2.30 | 2.3E−7 | 1.51 | 9.4E−7 | 1.25 | 1.5E−3 |
| | (I13R1) | | | | | | | | |
| 05 | IL15_HUMAN | 8 | P40933 | 2.86 | 1.6E−6 | 1.95 | 1.7E−6 | 1.42 | 6.9E−3 |
| | (IL_15) | | | | | | | | |
| 06 | IGKC_HUMAN | 5 | P01834 | −1.74 | 1.9E−6 | −0.54 | 6.3E−2 | −0.95 | 4.0E−3 |
| | (IGKC) | | | | | | | | |
| 07 | I13R2_HUMAN | 7 | Q14627 | 1.64 | 3.5E−6 | 1.20 | 1.5E−6 | 0.93 | 4.0E−3 |
| | (I13R2) | | | | | | | | |
| 08 | CSF1_HUMAN | 7 | P09603 | 2.74 | 1.6E−5 | 1.55 | 5.4E−4 | 1.59 | 5.2E−3 |
| | (CSF1) | | | | | | | | |
| 09 | IGF1R_HUMAN | 7 | P08069 | 2.69 | 5.9E−5 | 1.89 | 6.2E−5 | 1.70 | 4.6E−3 |
| | (IGF1R) | | | | | | | | |
| 10 | BCAM_HUMAN | 7 | P50895 | 2.35 | 8.9E−5 | 1.40 | 1.2E−3 | 1.70 | 1.6E−3 |
| | (BCAM) | | | | | | | | |
| 11 | CD166_HUMAN | 8 | Q13740 | 2.50 | 1.9E−6 | 1.35 | 3.5E−4 | 1.61 | 6.7E−4 |
| | (CD166) | | | | | | | | |
| 12 | OX2G_HUMAN | 7 | P41217 | −2.17 | 6.0E−5 | −1.69 | 9.7E−6 | −2.12 | 1.3E−5 |
| | (OX2G) | | | | | | | | |
| 13 | Exon A of | 5 | P08575 | 1.86 | 6.9E−5 | 0.92 | 9.1E−3 | 0.55 | 2.9E−1 |
| | PTPRC_HUMAN* | | | | | | | | |
| | (CD45RA*) | | | | | | | | |
| | *antibody recognizes | | | | | | | | |
| | all splice-variants | | | | | | | | |
| | comprising exon A. | | | | | | | | |
| 14 | TNR16_HUMAN | 6 | P08138 | 2.40 | 1.0E−4 | 1.75 | 6.9E−5 | 1.51 | 6.9E−3 |
| | (TNR16) | | | | | | | | |
| 15 | CXCR5_HUMAN | 6 | P32302 | 2.94 | 3.7E−4 | 2.06 | 4.7E−4 | 1.68 | 2.7E−2 |
| | (CXCR5) | | | | | | | | |
| 16 | CCL19_HUMAN | 5 | Q99731 | −1.93 | 6.9E−4 | −1.65 | 4.0E−5 | −2.08 | 2.6E−5 |
| | (CCL19) | | | | | | | | |
| 17 | LEUK_HUMAN | 5 | P16150 | 2.34 | 2.0E−3 | 1.65 | 2.2E−3 | 0.56 | 5.5E−1 |
| | (LEUK) | | | | | | | | |
| 18 | ICAM1_HUMAN | 4 | P05362 | 1.54 | 2.3E−5 | 0.19 | 6.0E−1 | 0.89 | 6.3E−3 |
| | (ICAM1) | | | | | | | | |
| 19 | TNFL4_HUMAN | 4 | P23510 | 1.95 | 6.0E−5 | 0.62 | 1.2E−1 | 0.18 | 8.0E−1 |
| | (TNFL4) | | | | | | | | |
| 20 | GLPB_HUMAN | 5 | P06028 | 2.23 | 3.4E−4 | 1.23 | 7.3E−3 | 0.53 | 4.8E−1 |
| | (GLPB) | | | | | | | | |
| 21 | IL2_HUMAN | 6 | P60568 | 2.72 | 3.4E−4 | 1.89 | 4.7E−4 | 1.80 | 7.5E−3 |
| | (IL2) | | | | | | | | |
| 22 | PD1L1_HUMAN | 6 | Q9NZQ7 | 2.31 | 3.7E−4 | 1.29 | 7.4E−3 | 1.56 | 6.9E−3 |
| | (PD1L1) | | | | | | | | |
| 23 | CCL27_HUMAN | 5 | Q9Y4X3 | 1.74 | 3.9E−4 | 1.15 | 1.1E−3 | 1.06 | 1.8E−2 |
| | (CCL27) | | | | | | | | |
| 24 | IL3_HUMAN | 4 | P08700 | 1.99 | 5.4E−4 | 1.11 | 9.6E−3 | 0.63 | 3.3E−1 |
| | (IL3) | | | | | | | | |
| 25 | HMGB1_HUMAN | 5 | P09429 | 1.62 | 1.9E−3 | 1.28 | 4.7E−4 | 1.35 | 3.8E−3 |
| | (HMGB1) | | | | | | | | |
| 26 | ALBU_HUMAN | 3 | P02768 | −1.63 | 2.3E−3 | −0.51 | 2.8E−1 | −0.95 | 5.7E−2 |
| | (ALBU) | | | | | | | | |
| 27 | SLAF1_HUMAN | 4 | Q13291 | 2.41 | 3.7E−4 | 0.79 | 1.6E−1 | 0.61 | 4.5E−1 |
| | (SLAF1) | | | | | | | | |
| 28 | CD47_HUMAN | 5 | Q08722 | 1.75 | 5.3E−4 | 1.28 | 4.3E−4 | 1.10 | 1.7E−2 |
| | (CD47) | | | | | | | | |
| 29 | TNFA_HUMAN | 4 | P01375 | 2.51 | 1.1E−3 | 1.07 | 8.5E−2 | 0.97 | 2.3E−1 |
| | (TNFA) | | | | | | | | |
| 30 | TLR3_HUMAN | 6 | O15455 | 2.76 | 1.8E−3 | 1.71 | 8.1E−3 | 2.44 | 1.6E−3 |
| | (TLR3) | | | | | | | | |
| 31 | TBB3_HUMAN | 4 | Q13509 | 1.77 | 5.4E−3 | 1.41 | 1.3E−3 | 1.64 | 3.2E−3 |
| | (TBB3) | | | | | | | | |
| 32 | S10A8_HUMAN* | 3 | P05109 | 1.59 | 1.3E−2 | 1.00 | 3.4E−2 | 0.65 | 3.3E−1 |
| | (S10A8*) | | | | | | | | |
| | *antibody recognizes | | | | | | | | |
| | both S10A8 as well | | | | | | | | |
| | as S10A9 | | | | | | | | |
| 33 | S10A9_HUMAN* | | P06702 | | | | | | |
| | (S10A9*) | | | | | | | | |
| | *antibody recognizes | | | | | | | | |
| | both S10A8 as well | | | | | | | | |
| | as S10A9 | | | | | | | | |
| 34 | TNF11_HUMAN | 4 | O14788 | 0.83 | 7.6E−5 | 0.44 | 2.0E−1 | 0.64 | 1.1E−1 |
| | (TNF11) | | | | | | | | |
| 35 | TFR1_HUMAN | 2 | P02786 | 1.23 | 1.4E−3 | 0.36 | 2.8E−1 | 0.18 | 7.3E−1 |
| | (TFR1) | | | | | | | | |
| 36 | TNR8_HUMAN | 4 | P28908 | −2.68 | 2.3E−3 | −1.16 | 1.0E−1 | −1.68 | 3.8E−2 |
| | (TNR8) | | | | | | | | |
| 37 | CEAM1_HUMAN*; | 4 | P13688 | 2.16 | 7.6E−3 | 1.20 | 5.0E−2 | 0.79 | 3.8E−1 |
| | (CEAM1*) | | | | | | | | |
| | *antibody recognizes | | | | | | | | |
| | CEAM1/3/5/6/8 | | | | | | | | |
| 38 | CEAM3_HUMAN*; | | | | | | | | |
| | (CEAM3*) | | | | | | | | |
| | *antibody recognizes | | | | | | | | |
| | CEAM1/3/5/6/8 | | | | | | | | |
| 39 | CEAM5_HUMAN* | | P40198 | | | | | | |
| | (CEAM5*) | | | | | | | | |
| | *antibody recognizes | | | | | | | | |
| | CEAM1/3/5/6/8 | | | | | | | | |
| 40 | CEAM6_HUMAN*; | | P06731 | | | | | | |
| | (CEAM6*) | | | | | | | | |
| | *antibody recognizes | | | | | | | | |
| | CEAM1/3/5/6/8 | | | | | | | | |
| 41 | CEAM8_HUMAN* | | P40199 | | | | | | |
| | (CEAM8*) | | | | | | | | |
| | *antibody recognizes | | | | | | | | |
| | CEAM1/3/5/6/8 | | | | | | | | |
| 42 | AREG_HUMAN | 4 | P31997 | 1.84 | 8.6E−3 | 1.05 | 4.5E−2 | 1.27 | 4.2E−2 |
| | (AREG) | | | | | | | | |
| 43 | HLAB_HUMAN* | 3 | P15514 | 1.53 | 8.7E−3 | 0.97 | 2.6E−2 | 0.86 | 1.2E−1 |
| | (HLA I*) | | | | | | | | |
| | *antibody recognizes | | | | | | | | |
| | all proteins belonging | | | | | | | | |
| | to the HLA class I | | | | | | | | |
| | histo−compatibility | | | | | | | | |
| | antigen. | | | | | | | | |
| 44 | CD81_HUMAN | 3 | P01889 | 1.54 | 3.8E−2 | 1.20 | 1.7E−2 | 0.60 | 4.5E−1 |
| | (CD81) | | | | | | | | |
| 45 | VEGFA_HUMAN | 3 | P60033 | 1.53 | 1.4E−2 | 1.14 | 8.1E−3 | 1.03 | 6.5E−2 |
| | (VEGFA) | | | | | | | | |
| 46 | CCL8_HUMAN | 4 | P15692 | −1.62 | 3.8E−2 | −1.48 | 3.0E−3 | −1.55 | 1.4E−2 |
| | (CCL8) | | | | | | | | |
| 47 | IL31_HUMAN | 3 | P80075 | −1.39 | 3.8E−2 | −1.12 | 1.1E−2 | −1.40 | 8.2E−3 |
| | (IL31) | | | | | | | | |
| 48 | K1C18_HUMAN | 3 | Q6EBC2 | 1.74 | 4.2E−2 | 1.20 | 4.5E−2 | 1.00 | 2.0E−1 |
| | (K1C18) | | | | | | | | |
| 49 | IL12B_HUMAN | 3 | P05783 | 1.52 | 4.2E−2 | 1.11 | 3.4E−2 | 1.19 | 6.3E−2 |
| | (IL12B) | | | | | | | | |
| 50 | ERBB2_HUMAN | 3 | P29460 | 1.56 | 1.1E−3 | 0.29 | 5.5E−1 | 0.64 | 1.9E−1 |
| | (ERBB2) | | | | | | | | |
| 51 | DPP4_HUMAN | 2 | P04626 | −1.57 | 5.4E−3 | −0.20 | 7.3E−1 | 0.23 | 7.6E−1 |
| | (DPP4) | | | | | | | | |
| 13 | Exon B of | 4 | P27487 | 2.15 | 6.2E−3 | 1.22 | 4.2E−2 | 1.03 | 1.9E−1 |
| | PTPRC_HUMAN* | | | | | | | | |
| | (CD45RB*) | | | | | | | | |
| | *antibody recognizes | | | | | | | | |
| | all splice−variants | | | | | | | | |
| | comprising exon B. | | | | | | | | |
| 52 | CD8A_HUMAN | 2 | P08575 | 1.34 | 5.3E−4 | 0.39 | 2.5E−1 | 0.30 | 5.5E−1 |
| | (CD8A) | | | | | | | | |
| 53 | IL1A_HUMAN | 3 | P01732 | 1.22 | 3.5E−3 | 0.87 | 3.1E−3 | 0.79 | 3.8E−2 |
| | (IL1A) | | | | | | | | |
| 54 | HAVR2_HUMAN | 2 | P01583 | −1.56 | 1.4E−2 | −0.56 | 2.9E−1 | −0.37 | 6.3E−1 |
| | (HAVR2) | | | | | | | | |
| 55 | IGLC1_HUMAN | 1 | Q8TDQ0 | −1.43 | 5.4E−3 | −0.35 | 4.5E−1 | −0.39 | 5.3E−1 |
| | (IGLC1) | | | | | | | | |
| 56 | TNFL6_HUMAN | 2 | P0CG04 | 1.32 | 1.3E−2 | 0.92 | 6.9E−3 | −0.32 | 6.2E−1 |
| | (TNFL6) | | | | | | | | |
| 57 | CCL2_HUMAN | 1 | P48023 | 1.43 | 3.7E−2 | 0.55 | 3.0E−1 | 1.47 | 6.9E−3 |
| | (CCL2) | | | | | | | | |
| 58 | TNR5_HUMAN | 0 | P13500 | 0.61 | 4.0E−2 | 0.44 | 3.4E−2 | 0.77 | 9.3E−4 |
| | (TNR5) | | | | | | | | |
| 59 | ADIPO_HUMAN | 1 | P25942 | −1.04 | 4.1E−2 | −0.24 | 6.0E−1 | −0.42 | 4.3E−1 |
| | (ADIPO) | | | | | | | | |
| 60 | CD4_HUMAN | 3 | Q15848 | 1.09 | 6.7E−3 | 1.62 | 1.2E−9 | 1.32 | 9.8E−5 |
| | (CD4) | | | | | | | | |
| 61 | CD38_HUMAN | 1 | P01730 | 1.04 | 3.9E−2 | 0.25 | 5.9E−1 | 0.29 | 6.0E−1 |
| | (CD38) | | | | | | | | |
| 62 | IL20_HUMAN | 1 | P28907 | 1.34 | 4.8E−2 | 0.39 | 4.9E−1 | −0.33 | 6.7E−1 |
| | (IL20) | | | | | | | | |

TABLE 3
Additional biomarkers differential only in medium phase
(not overlapping with acute phase/early phase)

| Time-point of differentiating measurement: | | | medium phase | |
|---|---|---|---|---|
| SEQ ID | | Uniprot | | adj. p- |
| No. | Uniprot entry name | accession | logFC | Val |
| 63 | HGF_HUMAN | P14210 | 1.32 | 1.4E−4 |
| | (HGF) | | | |
| 64 | CD276_HUMAN | Q5ZPR3 | 1.15 | 1.0E−3 |
| | (CD276) | | | |
| 65 | CTLA4_HUMAN | P16410 | 1.13 | 4.6E−2 |
| | (CTLA4) | | | |
| 66 | INHBA_HUMAN | P08476 | 1.09 | 5.3E−4 |
| | (INHBA) | | | |
| 67 | TR13B_HUMAN | O14836 | 0.90 | 4.1E−2 |
| | (TR13B) | | | |
| 68 | OSTP_HUMAN | P10451 | 0.60 | 4.0E−2 |
| | (OSTP) | | | |
| 69 | CCL28_HUMAN | Q9NRJ3 | −0.61 | 2.2E−2 |
| | (CCL28) | | | |
| 70 | IFNG_HUMAN | P01579 | −0.69 | 5.0E−2 |
| | (IFNG) | | | |
| 71 | CO3_HUMAN | P01024 | −0.89 | 4.5E−2 |
| | (CO3) | | | |
| 72 | TNFB_HUMAN | P01374 | −1.14 | 1.5E−3 |
| | (TNFB) | | | |

TABLE 4
Additional biomarkers differential in medium and late
phase (not overlapping with acute phase/early phase)

| Time-point of differentiating measurement: | | | medium phase | | late phase | |
|---|---|---|---|---|---|---|
| SEQ ID | Uniprot | Uniprot | | adj. p- | | adj. p- |
| No. | entry name | accession | logFC | Val | logFC | Val |
| 73 | BTLA_HUMAN | Q7Z6A9 | 1.27 | 1.6E−3 | 1.09 | 3.8E−2 |
| | (BTLA) | | | | | |
| 74 | CD3D_HUMAN; | P04234 | 1.04 | 2.1E−2 | 1.40 | 6.9E−3 |
| | (CD3deg*) | | | | | |
| | *antibody recognizes | | | | | |
| | all variants | | | | | |
| | of CD3. | | | | | |
| 75 | CD3E_HUMAN* | P07766 | | | | |
| | (CD3deg*) | | | | | |
| | *antibody recognizes | | | | | |
| | all variants | | | | | |
| | of CD3. | | | | | |
| 76 | CD3G_HUMAN* | P09693 | | | | |
| | (CD3deg*) | | | | | |
| | *antibody recognizes | | | | | |
| | all variants | | | | | |
| | of CD3. | | | | | |
| 77 | TNR6_HUMAN | P25445 | 0.98 | 3.9E−3 | 0.92 | 1.4E−2 |
| | (TNR6) | | | | | |
| 78 | SLAF8_HUMAN | Q9P0V8 | 0.82 | 4.3E−2 | 1.01 | 3.5E−2 |
| | (SLAF8) | | | | | |
| 79 | NCAM1_HUMAN | P13591 | 0.63 | 2.5E−3 | 0.68 | 9.2E−3 |
| | (NCAM1) | | | | | |
| 80 | IFNL2_HUMAN | Q8IZJ0 | 0.57 | 4.5E−2 | 0.90 | 5.2E−3 |
| | (IFNL2) | | | | | |
| 81 | ANGP4_HUMAN | Q9Y264 | 0.52 | 2.2E−2 | 0.61 | 2.5E−2 |
| | (ANGP4) | | | | | |
| 82 | CCL7_HUMAN | P80098 | −0.54 | 4.5E−2 | −0.70 | 2.6E−2 |
| | (CCL7) | | | | | |
| 83 | DPA1_HUMAN* | P20036 | −0.57 | 4.5E−2 | −1.08 | 7.0E−4 |
| | (HLA-II*) | | | | | |
| | *antibody recognizes | | | | | |
| | all proteins belonging | | | | | |
| | to the HLA class II | | | | | |
| | histo-compatibility | | | | | |
| | antigen. | | | | | |
| 84 | AMPN_HUMAN | P15144 | −0.59 | 2.6E−2 | −0.76 | 1.4E−2 |
| | (AMPN) | | | | | |
| 85 | TSLP_HUMAN | Q969D9 | −0.98 | 3.6E−2 | −1.29 | 2.0E−2 |
| | (TSLP) | | | | | |
| 86 | CXL13_HUMAN | O43927 | −1.54 | 1.6E−3 | −1.76 | 4.5E−3 |
| | (CXL13) | | | | | |

TABLE 5
Additional biomarkers differential only in late phase
(not overlapping with acute phase/early phase)

| Time-point of differentiating measurement: | | | late phase | |
|---|---|---|---|---|
| SEQ ID | | Uniprot | | adj. p- |
| No. | Uniprot entry name | accession | logFC | Val |
| 87 | PLF4 HUMAN | P02776 | 1.67 | 4.9E−2 |
| | (PLF4) | | | |
| 88 | TR11B_HUMAN | O00300 | 1.59 | 1.8E−4 |
| | (TR11B) | | | |
| 89 | IL22_HUMAN | Q9GZX6 | 1.33 | 3.4E−2 |
| | (IL22) | | | |
| Formula | (CD15*) | - no | 1.29 | 1.8E−2 |
| I | *CD15 is no protein, | protein - | | |
| | but a tetrasaccharide | | | |
| | carbohydrate. | | | |
| 90 | ITA2B_HUMAN | P08514 | 1.24 | 1.4E−2 |
| | (ITA2B) | | | |
| 91 | IL2RA_HUMAN | P01589 | 1.14 | 4.3E−2 |
| | (IL2RA) | | | |
| 92 | FLT3_HUMAN | P36888 | 1.10 | 4.0E−3 |
| | (FLT3) | | | |
| 93 | LIF_HUMAN | P15018 | 1.06 | 1.9E−2 |
| | (LIF) | | | |
| 94 | PRIO_HUMAN | P04156 | 1.02 | 2.5E−2 |
| | (PRIO) | | | |
| 95 | CD72_HUMAN | P21854 | 0.77 | 3.9E−3 |
| | (CD72) | | | |
| 96 | HLAA_HUMAN* | P04439 | 0.77 | 4.6E−3 |
| | (HLA-ABC*) | | | |
| | *antibody recognizes | | | |
| | HLA-A, B and C. | | | |
| 43 | HLAB_HUMAN* | P01889 | | |
| | (HLA-ABC*) | | | |
| | *antibody recognizes | | | |
| | HLA-A, B and C. | | | |
| 97 | HLAC_HUMAN* | P10321 | | |
| | (HLA-ABC*) | | | |
| | *antibody recognizes | | | |
| | HLA-A, B and C. | | | |
| 98 | ITAM_HUMAN | P11215 | 0.75 | 6.9E−3 |
| | (ITAM) | | | |
| 99 | ONCM_HUMAN | P13725 | 0.75 | 2.7E−2 |
| | (ONCM) | | | |
| 100 | NP1L4 HUMAN | Q99733 | 0.72 | 4.2E−2 |
| | (NP1L4) | | | |
| 101 | AGRE5_HUMAN | P48960 | −0.65 | 2.5E−2 |
| | (AGRE5) | | | |
| 102 | EGLN_HUMAN | P17813 | −0.78 | 1.4E−2 |
| | (EGLN) | | | |
| 103 | CXCL9_HUMAN | Q07325 | −1.03 | 3.3E−5 |
| | (CXCL9) | | | |
| 104 | CCL3_HUMAN | P10147 | −1.16 | 3.6E−2 |
| | (CCL3) | | | |
| 27 | SLAF1_HUMAN | Q13291 | −1.37 | 3.8E−2 |
| | (SLAF1) * | | | |
| | (*also found in acute | | | |
| | phase) | | | |
| 105 | SCRB2_HUMAN | Q14108 | −1.44 | 2.5E−2 |
| | (SCRB2) | | | |

LogFC-values and adjusted p-values are shown in tables 1-4 as well. Positive log FC-values indicate that the respective biomarker is upregulated in CS-patients as compared to MM-patients. Negative log FC-values indicate that the respective biomarker is downregulated in CS-patients as compared to MM-patients. Please note that the LogFC-values and p-values differ between the three time-phases of measurement (acute phase from −3 days prior to onset of first symptoms up to 9 days, medium phase 10-21 days; late phase >21 days).

The Uniprot entry name and the Uniprot accession number are indicated if applicable. Throughout the application the “short name” of the protein was used (depicted in brackets in the tables).

In one case (CD15) there is no Uniprot entry name and accession number, since CD15 is no protein, but a tetrasaccharide carbohydrate.

CD15 is depicted by formula I:

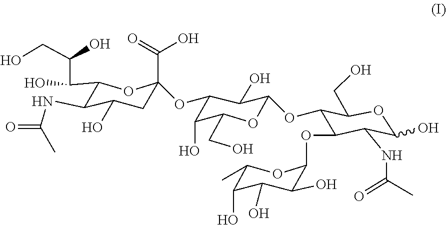 [SMILES: CC(=O)NC1C(O)OC(CO)C(OC2OC(CO)C(O)C(OC3(C(=O)O)CC(O)C(NC(C)=O)C([C@H](O)[C@H](O)CO)O3)C2O)C1OC1OC(C)C(O)C(O)C1O]

The invention also encompasses all isoforms, fragments and variants of the canonical proteins as listed in Uniprot under the accession number in the section “similar proteins”. This section provides links to proteins that are similar to the protein sequence(s) described in this entry at different levels of sequence identity thresholds (100%, 90% and 50%) based on their membership in UniProt Reference Clusters (UniRef).

### Example 2—Identification of Important Subgroups of the Biomarkers De-Regulated in CS-Patients Depending on Certain Aspects

In order to identify subgroups of the biomarkers of interest for certain aspects of a severe or critical course of a COVID-19 disease, the following tables 6-10 have been generated:

TABLE 6
Selected biomarkers related to specific biological processes
with differential abundance in acute CS vs MM.

| | |
|---|---|
| GO:0002684: positive regulation | CCL19 (−), CCL2 (+), CCL8 (−), PD1L1 (+), CD28 (+), CD38 (+), |
| of immune system process | CD4 (+), TNR5 (+), CD47 (+), CD81 (+), CSF1 (+), DPP4 (−), |
| | HAVR2 (−), HMGB1 (+), ICAM1 (+), NA * IL12B (+), I13R2 (+), |
| | IL15 (+), IL1A (+), IL2 (+), IL20 (+), PTPRC (+), LEUK (+), TFR1 |
| | (+), TGFB2 (+), TLR3 (+), TNFA (+), TNF11 (+), TNFL4 (+), |
| | VEGFA (+) |
| GO:0008284: positive regulation | AREG (+), CCL19 (−), PD1L1 (+), CD28 (+), CD38 (+), CD4 (+), |
| of cell population proliferation | TNR5 (+), CD47 (+), CD81 (+), CSF1 (+), DPP4 (−), ERBB2 (+), |
| | TNFL6 (+), FGF2 (+), HAVR2 (−), HMGB1 (+), IGF1R (+), IL12B |
| | (+), IL15 (+), IL2 (+), IL3 (+), PTPRC (+), SLAF1 (+), TFR1 (+), |
| | TGFB2 (+), TNFA (+), TNFL4 (+), VEGFA (+) |
| GO:0045785: positive regulation | CCL19 (−), CCL2 (+), PD1L1 (+), CD28 (+), CD4 (+), CD47 (+), |
| of cell adhesion | CSF1 (+), DPP4 (−), ERBB2 (+), HAVR2 (−), HMGB1 (+), ICAM1 |
| | (+), IL12B (+), IL15 (+), IL2 (+), PTPRC (+), TFR1 (+), TGFB2 (+), |
| | TNFA (+), TNF11 (+), TNFL4 (+), VEGFA (+) |
| GO:0051251: positive regulation | CCL19 (−), CCL2 (+), PD1L1 (+), CD28 (+), CD38 (+), CD4 (+), |
| of lymphocyte activation | TNR5 (+), CD47 (+), CD81 (+), DPP4 (−), HAVR2 (−), HMGB1 (+), |
| | IL12B (+), IL15 (+), IL2 (+), PTPRC (+), TFR1 (+), TNF11 (+), |
| | TNFL4 (+) |
| GO:0048585: negative regulation | ADIPO (−), CCL2 (+), CEAM1 (+), ERBB2 (+), TNFL6 (+), FGF2 |
| of response to stimulus | (+), HAVR2 (−), ICAM1 (+), IGF1R (+), IL12B (+), I13R2 (+), IL1A |
| | (+), IL2 (+), PTPRC (+), SLAF1 (+), TGFB2 (+), TLR3 (+), TNFA |
| | (+), TNFL4 (+) |
| GO:1903039: positive regulation | CCL19 (−), CCL2 (+), PD1L1 (+), CD28 (+), CD4 (+), CD47 (+), |
| of leukocyte cell-cell adhesion | DPP4 (−), HAVR2 (−), HMGB1 (+), ICAM1 (+), IL12B (+), IL15 (+), |
| | IL2 (+), PTPRC (+), TFR1 (+), TNFA (+), TNF11 (+), TNFL4 (+) |
| GO:0050778: positive regulation | CCL19 (−), PD1L1 (+), CD28 (+), CD38 (+), CD4 (+), TNR5 (+), |
| of immune response | HAVR2 (−), HMGB1 (+), NA * IL12B (+), IL15 (+), IL2 (+), PTPRC |
| | (+), TFR1 (+), TGFB2 (+), TLR3 (+), TNFA (+), TNFL4 (+) |
| GO:0001819: positive regulation | ADIPO (−), CCL19 (−), PD1L1 (+), CD28 (+), CD4 (+), TNR5 (+), |
| of cytokine production | HAVR2 (−), HMGB1 (+), IL12B (+), IL15 (+), IL1A (+), IL2 (+), |
| | LEUK (+), TLR3 (+), TNFA (+), TNR8 (−), TNFL4 (+) |
| GO:0002683: negative regulation | ADIPO (−), CCL2 (+), OX2G (−), PD1L1 (+), CEAM1 (+), ERBB2 |
| of immune system process | (+), HAVR2 (−), HMGB1 (+), IL12B (+), I13R2 (+), IL2 (+), PTPRC |
| | (+), LEUK (+), TGFB2 (+), TLR3 (+), TNFA (+), TNFL4 (+) |
| GO:0043410: positive regulation | CCL19 (−), CCL2 (+), CCL8 (−), CD4 (+), TNR5 (+), CD81 (+), |
| of MAPK cascade | ERBB2 (+), FGF2 (+), HAVR2 (−), HMGB1 (+), ICAM1 (+), SLAF1 |
| | (+), TGFB2 (+), TLR3 (+), TNFA (+), TNF11 (+), VEGFA (+) |
| GO:0032946: positive regulation | CCL19 (−), PD1L1 (+), CD28 (+), CD38 (+), CD4 (+), TNR5 (+), |
| of mononuclear cell proliferation | CD81 (+), CSF1 (+), HAVR2 (−), HMGB1 (+), IL12B (+), IL15 (+), |
| | IL2 (+), PTPRC (+), TFR1 (+), TNFL4 (+) |
| GO:0050870: positive regulation | CCL19 (−), CCL2 (+), PD1L1 (+), CD28 (+), CD4 (+), CD47 (+), |
| of T cell activation | DPP4 (−), HAVR2 (−), HMGB1 (+), IL12B (+), IL15 (+), IL2 (+), |
| | PTPRC (+), TFR1 (+), TNF11 (+), TNFL4 (+) |
| GO:0050671: positive regulation | CCL19 (−), PD1L1 (+), CD28 (+), CD38 (+), CD4 (+), TNR5 (+), |
| of lymphocyte proliferation | CD81 (+), HAVR2 (−), HMGB1 (+), IL12B (+), IL15 (+), IL2 (+), |
| | PTPRC (+), TFR1 (+), TNFL4 (+) |
| GO:0032103: positive regulation | CCL19 (−), CD28 (+), CD47 (+), CSF1 (+), FGF2 (+), HAVR2 (−), |
| of response to external stimulus | HMGB1 (+), IL12B (+), IL15 (+), IL2 (+), TLR3 (+), TNFA (+), |
| | TNF11 (+), TNFL4 (+), VEGFA (+) |
| GO:0042102: positive regulation | CCL19 (−), PD1L1 (+), CD28 (+), CD4 (+), HAVR2 (−), HMGB1 (+), |
| of T cell proliferation | IL12B (+), IL15 (+), IL2 (+), PTPRC (+), TFR1 (+), TNFL4 (+) |
| GO:0070374: positive regulation | CCL19 (−), CCL2 (+), CCL8 (−), CD4 (+), FGF2 (+), HAVR2 (−), |
| of ERK1 and ERK2 cascade | HMGB1 (+), ICAM1 (+), SLAF1 (+), TNFA (+), TNF11 (+), VEGFA |
| | (+) |
| GO:0043065: positive regulation | ADIPO (−), CCL2 (+), PD1L1 (+), TNR5 (+), TNFL6 (+), HMGB1 |
| of apoptotic process | (+), IL12B (+), TNR16 (+), TGFB2 (+), TLR3 (+), TNFA (+), TNR8 |
| | (−) |
| GO:1902107: positive regulation | CCL19 (−), CD4 (+), CSF1 (+), HMGB1 (+), IL12B (+), IL15 (+), |
| of leukocyte differentiation | IL2 (+), IL20 (+), TNFA (+), TNF11 (+), TNFL4 (+) |
| GO:0031349: positive regulation | CD28 (+), CD47 (+), HAVR2 (−), HMGB1 (+), IL12B (+), IL15 (+), |
| of defense response | IL2 (+), TLR3 (+), TNFA (+), TNF11 (+), TNFL4 (+) |
| GO:0002824: positive regulation | CCL19 (−), PD1L1 (+), CD28 (+), CD4 (+), TNR5 (+), IL12B (+), |
| of adaptive immune response based | IL2 (+), TFR1 (+), TNFA (+), TNFL4 (+) |
| on somatic recombination of immune | |
| receptors built from immunoglobulin | |
| superfamily domains | |
| GO:0002695: negative regulation | OX2G (−), PD1L1 (+), CEAM1 (+), ERBB2 (+), HAVR2 (−), HMGB1 |
| of leukocyte activation | (+), I13R2 (+), IL2 (+), LEUK (+), TNFL4 (+) |
| GO:0001818: negative regulation | ADIPO (−), PD1L1 (+), CEAM1 (+), HAVR2 (−), HMGB1 (+), IL12B |
| of cytokine production | (+), SLAF1 (+), TGFB2 (+), TNFA (+), TNFL4 (+) |
| GO:0098542: defense response | CD4 (+), TNR5 (+), HAVR2 (−), NA * IL12B (+), PTPRC (+), LEUK |
| to other organism | (+), TLR3 (+), TNFA (+), TNFL4 (+) |
| GO:0050871: positive regulation | CD28 (+), CD38 (+), TNR5 (+), CD81 (+), NA * IL2 (+), PTPRC |
| of B cell activation | (+), TFR1 (+), TNFL4 (+) |
| GO:0050729: positive regulation | CD28 (+), CD47 (+), IL12B (+), IL15 (+), IL2 (+), TLR3 (+), TNFA |
| of inflammatory response | (+), TNF11 (+), TNFL4 (+) |
| GO:0002687: positive regulation | CCL19 (−), CCL8 (−), CSF1 (+), HMGB1 (+), ICAM1 (+), IL1A (+), |
| of leukocyte migration | LEUK (+), TNFA (+), VEGFA (+) |
| GO:0050777: negative regulation | CEAM1 (+), HAVR2 (−), IL12B (+), I13R2 (+), IL2 (+), PTPRC (+), |
| of immune response | TGFB2 (+), TNFA (+), TNFL4 (+) |
| GO:0002699: positive regulation | CCL19 (−), CD28 (+), TNR5 (+), IL12B (+), I13R2 (+), IL2 (+), |
| of immune effector process | TFR1 (+), TNFA (+), TNFL4 (+) |
| GO:0050868: negative regulation | PD1L1 (+), CEAM1 (+), ERBB2 (+), HAVR2 (−), HMGB1 (+), IL2 |
| of T cell activation | (+), LEUK (+), TNFL4 (+) |
| GO:0002698: negative regulation | CEAM1 (+), HAVR2 (−), I13R2 (+), IL2 (+), PTPRC (+), TGFB2 |
| of immune effector process | (+), TNFA (+), TNFL4 (+) |
| GO:0043123: positive regulation | ADIPO (−), CCL19 (−), CD4 (+), TNR5 (+), TNFL6 (+), TLR3 (+), |
| of I-kappaB kinase/NF-kappaB | TNFA (+), TNF11 (+) |
| signaling | |
| GO:0032874: positive regulation | CCL19 (−), HMGB1 (+), SLAF1 (+), TGFB2 (+), TLR3 (+), TNFA |
| of stress-activated MAPK cascade | (+), TNF11 (+), VEGFA (+) |
| GO:0007204: positive regulation | CCL19 (−), CD38 (+), CD4 (+), TNFL6 (+), FGF2 (+), HMGB1 (+), |
| of cytosolic calcium ion | IL2 (+), PTPRC (+) |
| concentration | |
| GO:0043406: positive regulation | CCL19 (−), TNR5 (+), CD81 (+), ERBB2 (+), FGF2 (+), TNFA (+), |
| of MAP kinase activity | TNF11 (+), VEGFA (+) |
| GO:0009615: response to virus | CCL19 (−), CCL8 (−), TNR5 (+), IL12B (+), PTPRC (+), TLR3 (+), |
| | TNFA (+), TNFL4 (+) |
| GO:0032760: positive regulation | CCL19 (−), HAVR2 (−), HMGB1 (+), IL12B (+), LEUK (+), TLR3 |
| of tumor necrosis factor production | (+), TNR8 (−) |
| GO:0002708: positive regulation | CD28 (+), TNR5 (+), IL12B (+), IL2 (+), TFR1 (+), TNFA (+), |
| of lymphocyte mediated immunity | TNFL4 (+) |
| GO:0045639: positive regulation | CD4 (+), CSF1 (+), HMGB1 (+), IL12B (+), IL20 (+), TNFA (+), |
| of myeloid cell differentiation | TNF11 (+) |
| GO:1902106: negative regulation | ADIPO (−), CEAM1 (+), ERBB2 (+), HMGB1 (+), IL2 (+), TLR3 (+), |
| of leukocyte differentiation | TNFL4 (+) |
| GO:0032675: regulation of | HAVR2 (−), HMGB1 (+), IL1A (+), SLAF1 (+), TLR3 (+), TNFA (+), |
| interleukin-6 production | TNFL4 (+) |
| GO:0070555: response to | CCL19 (−), CCL2 (+), CCL8 (−), CD38 (+), TNR5 (+), ICAM1 (+), |
| interleukin-1 | IL1A (+) |
| GO:0019058: viral life cycle | CCL2 (+), CD4 (+), CD81 (+), DPP4 (−), ICAM1 (+), SLAF1 (+), |
| | TFR1 (+) |
| GO:1904018: positive regulation | TNR5 (+), CEAM1 (+), FGF2 (+), HMGB1 (+), IL1A (+), TLR3 (+), |
| of vasculature development | VEGFA (+) |
| GO:0032735: positive regulation | CCL19 (−), TNR5 (+), HMGB1 (+), IL12B (+), TLR3 (+), TNFL4 (+) |
| of interleukin-12 production | |
| GO:0030890: positive regulation | CD38 (+), TNR5 (+), CD81 (+), IL2 (+), PTPRC (+), TFR1 (+) |
| of B cell proliferation | |
| GO:0002763: positive regulation | CD4 (+), CSF1 (+), IL12B (+), IL20 (+), TNFA (+), TNF11 (+) |
| of myeloid leukocyte | |
| differentiation | |
| GO:0032722: positive regulation | ADIPO (−), HAVR2 (−), HMGB1 (+), IL1A (+), TLR3 (+), TNFA (+) |
| of chemokine production | |
| GO:0032729: positive regulation | HAVR2 (−), IL12B (+), IL2 (+), TLR3 (+), TNFA (+), TNFL4 (+) |
| of interferon-gamma production | |
| GO:1901224: positive regulation | CCL19 (−), HAVR2 (−), HMGB1 (+), IL12B (+), TLR3 (+), TNFA (+) |
| of NIK/NF-kappaB signaling | |
| GO:0070664: negative regulation | CCL8 (−), PD1L1 (+), ERBB2 (+), HAVR2 (−), IL2 (+), LEUK (+) |
| of leukocyte proliferation | |
| GO:0046718: viral entry into | CD4 (+), CD81 (+), DPP4 (−), ICAM1 (+), SLAF1 (+), TFR1 (+) |
| host cell | |
| GO:0045672: positive regulation | CSF1 (+), IL12B (+), IL20 (+), TNFA (+), TNF11 (+) |
| of osteoclast differentiation | |
| GO:0032733: positive regulation | PD1L1 (+), CD28 (+), HMGB1 (+), IL12B (+), TNFL4 (+) |
| of interleukin-10 production | |
| GO:0002823: negative regulation | CEAM1 (+), HAVR2 (−), IL2 (+), PTPRC (+), TNFL4 (+) |
| of adaptive immune response | |
| based on somatic recombination | |
| of immune receptors built from | |
| immunoglobulin superfamily | |
| domains | |
| GO:0046631: alpha-beta T cell | HMGB1 (+), IL12B (+), IL15 (+), LEUK (+), TNFL4 (+) |
| activation | |
| GO:0032660: regulation of | IL12B (+), IL15 (+), IL2 (+), TNFL4 (+) |
| interleukin-17 production | |
| GO:0002825: regulation of T- | CCL19 (−), HAVR2 (−), IL12B (+), TNFL4 (+) |
| helper 1 type immune response | |
| GO:0046636: negative regulation | PD1L1 (+), HMGB1 (+), IL2 (+), TNFL4 (+) |
| of alpha-beta T cell activation | |
| GO:0035710: CD4-positive, | HMGB1 (+), IL12B (+), LEUK (+), TNFL4 (+) |
| alpha-beta T cell activation | |
| GO:0071639: positive regulation | ADIPO (−), HMGB1 (+), IL1A (+) |
| of monocyte chemotactic protein- | |
| 1 production | |
| GO:0045086: positive regulation | CD28 (+), CD4 (+), IL1A (+) |
| of interleukin-2 biosynthetic | |
| process | |
| GO:0042088: T-helper 1 type | HMGB1 (+), IL12B (+), LEUK (+) |
| immune response | |
| GO:0019221: cytokine-mediated | CCL19 (−), CCL2 (+), CCL8 (−), CD4 (+), TNR5 (+), CEAM1 (+), |
| signaling pathway | CSF1 (+), CXCR5 (+), TNFL6 (+), FGF2 (+), ICAM1 (+), IL12B |
| | (+), I13R1 (+), I13R2 (+), IL15 (+), IL1A (+), IL2 (+), IL20 (+), IL3 |
| | (+), IL31 (−), K1C18 (+), TNR16 (+), TNFA (+), TNR8 (−), TNF11 |
| | (+), TNFL4 (+), VEGFA (+) |
| GO:0002694: regulation of | CCL19 (−), CCL2 (+), OX2G (−), PD1L1 (+), CD28 (+), CD38 (+), |
| leukocyte activation | CD4 (+), TNR5 (+), CD47 (+), CD81 (+), CEAM1 (+), DPP4 (−), |
| | ERBB2 (+), HAVR2 (−), HMGB1 (+), IL12B (+), I13R2 (+), IL15 (+), |
| | IL2 (+), PTPRC (+), LEUK (+), TFR1 (+), TNF11 (+), TNFL4 (+) |
| GO:0051249: regulation of | CCL19 (−), CCL2 (+), PD1L1 (+), CD28 (+), CD38 (+), CD4 (+), |
| lymphocyte activation | TNR5 (+), CD47 (+), CD81 (+), CEAM1 (+), DPP4 (−), ERBB2 (+), |
| | HAVR2 (−), HMGB1 (+), IL12B (+), IL15 (+), IL2 (+), PTPRC (+), |
| | LEUK (+), TFR1 (+), TNF11 (+), TNFL4 (+) |
| GO:0010941: regulation of cell | ADIPO (−), ALBU (−), CCL19 (−), CCL2 (+), PD1L1 (+), CD28 (+), |
| death | CD38 (+), TNR5 (+), TNFL6 (+), FGF2 (+), HMGB1 (+), ICAM1 |
| | (+), IGF1R (+), IL12B (+), IL1A (+), IL2 (+), K1C18 (+), TNR16 (+), |
| | TGFB2 (+), TLR3 (+), TNFA (+), TNR8 (−), VEGFA (+) |
| GO:0051252: regulation of | ADIPO (−), CD28 (+), CD38 (+), CD4 (+), TNR5 (+), CD81 (+), |
| RNA metabolic process | ERBB2 (+), TNFL6 (+), FGF2 (+), HAVR2 (−), HMGB1 (+), ICAM1 |
| | (+), IL1A (+), IL2 (+), TNR16 (+), TGFB2 (+), TLR3 (+), TNFA (+), |
| | TNF11 (+), TNFL4 (+), VEGFA (+) |
| GO:0001817: regulation of | ADIPO (−), CCL19 (−), PD1L1 (+), CD28 (+), CD4 (+), TNR5 (+), |
| cytokine production | CEAM1 (+), HAVR2 (−), HMGB1 (+), IL12B (+), IL15 (+), IL1A (+), |
| | IL2 (+), SLAF1 (+), LEUK (+), TGFB2 (+), TLR3 (+), TNFA (+), |
| | TNR8 (−), TNFL4 (+) |
| GO:0050863: regulation of T | CCL19 (−), CCL2 (+), PD1L1 (+), CD28 (+), CD4 (+), CD47 (+), |
| cell activation | CEAM1 (+), DPP4 (−), ERBB2 (+), HAVR2 (−), HMGB1 (+), IL12B |
| | (+), IL15 (+), IL2 (+), PTPRC (+), LEUK (+), TFR1 (+), TNF11 (+), |
| | TNFL4 (+) |
| GO:0032944: regulation of | CCL19 (−), PD1L1 (+), CD28 (+), CD38 (+), CD4 (+), TNR5 (+), |
| mononuclear cell proliferation | CD81 (+), CSF1 (+), ERBB2 (+), HAVR2 (−), HMGB1 (+), IL12B |
| | (+), IL15 (+), IL2 (+), PTPRC (+), LEUK (+), TFR1 (+), TNFL4 (+) |
| GO:0051254: positive regulation | CD28 (+), CD38 (+), CD4 (+), TNR5 (+), CD81 (+), ERBB2 (+), |
| of RNA metabolic process | FGF2 (+), HMGB1 (+), IL1A (+), IL2 (+), TNR16 (+), TGFB2 (+), |
| | TLR3 (+), TNFA (+), TNF11 (+), VEGFA (+) |
| GO:0070372: regulation of | ADIPO (−), CCL19 (−), CCL2 (+), CCL8 (−), CD4 (+), CEAM1 (+), |
| ERK1 and ERK2 cascade | ERBB2 (+), FGF2 (+), HAVR2 (−), HMGB1 (+), ICAM1 (+), SLAF1 |
| | (+), TNFA (+), TNF11 (+), VEGFA (+) |
| GO:0034612: response to tumor | ADIPO (−), CCL19 (−), CCL2 (+), CCL8 (−), TNR5 (+), ICAM1 (+), |
| necrosis factor | K1C18 (+), TNR16 (+), TNFA (+), TNR8 (−), TNF11 (+), TNFL4 (+) |
| GO:0060429: epithelium | AREG (+), CEAM1 (+), CSF1 (+), FGF2 (+), ICAM1 (+), K1C18 |
| development | (+), TGFB2 (+), TNF11 (+), VEGFA (+) |
| GO:0032649: regulation of | PD1L1 (+), HAVR2 (−), HMGB1 (+), IL12B (+), IL2 (+), TLR3 (+), |
| interferon-gamma production | TNFA (+), TNFL4 (+) |
| GO:0051726: regulation of cell | CCL2 (+), CD28 (+), CXCR5 (+), IL12B (+), IL1A (+), PTPRC (+), |
| cycle | TGFB2 (+), TNFA (+) |
| GO:0032655: regulation of | CCL19 (−), TNR5 (+), HMGB1 (+), IL12B (+), SLAF1 (+), TLR3 (+), |
| interleukin-12 production | TNFL4 (+) |
| GO:0046328: regulation of JNK | CCL19 (−), HMGB1 (+), IGF1R (+), SLAF1 (+), TLR3 (+), TNFA |
| cascade | (+), TNF11 (+) |
| GO:0070848: response to growth | CCL2 (+), ERBB2 (+), TNFL6 (+), FGF2 (+), TNR16 (+), TGFB2 |
| factor | (+), VEGFA (+) |
| GO:0051607: defense response to | TNR5 (+), IL12B (+), PTPRC (+), TLR3 (+) |
| virus | |
| GO:0032753: positive regulation | CD28 (+), HAVR2 (−), TNFL4 (+) |
| of interleukin-4 production | |
| GO:0042116: macrophage activation | HAVR2 (−), HMGB1 (+), TLR3 (+) |
| GO:1903902: positive regulation | CD28 (+), CD4 (+) |
| of viral life cycle | |

TABLE 7
Selected biomarkers related to specific cellular components
with differential abundance in acute CS vs MM.

| | |
|---|---|
| GO:0071944: cell periphery | CD166 (+), BCAM (+), OX2G (−), PD1L1 (+), CD28 (+), CD38 (+), |
| | CD4 (+), TNR5 (+), CD47 (+), CD81 (+), CD8A (+), CEAM1 (+), |
| | CSF1 (+), CXCR5 (+), DPP4 (−), ERBB2 (+), TNFL6 (+), GLPB |
| | (+), HAVR2 (−), HMGB1 (+), ICAM1 (+), IGF1R (+), I13R1 (+), |
| | K1C18 (+), TNR16 (+), PTPRC (+), SLAF1 (+), LEUK (+), TFR1 |
| | (+), TLR3 (+), TNFA (+), TNR8 (−), TNF11 (+), TNFL4 (+) |
| GO:0005576: extracellular | ADIPO (−), ALBU (−), AREG (+), CCL19 (−), CCL2 (+), CCL27 (+), |
| region | CCL8 (−), PD1L1 (+), TNR5 (+), CD47 (+), CD81 (+), CD8A (+), |
| | CSF1 (+), DPP4 (−), TNFL6 (+), FGF2 (+), HMGB1 (+), ICAM1 |
| | (+), IL12B (+), I13R2 (+), IL15 (+), IL1A (+), IL2 (+), IL20 (+), IL3 |
| | (+), IL31 (−), TNR16 (+), LEUK (+), TFR1 (+), TGFB2 (+), TNFA |
| | (+), TNF11 (+), TNFL4 (+), VEGFA (+) |
| GO:0005886: plasma | CD166 (+), BCAM (+), OX2G (−), PD1L1 (+), CD28 (+), CD38 (+), |
| membrane | CD4 (+), TNR5 (+), CD47 (+), CD81 (+), CD8A (+), CEAM1 (+), |
| | CSF1 (+), CXCR5 (+), DPP4 (−), ERBB2 (+), TNFL6 (+), GLPB |
| | (+), HAVR2 (−), HMGB1 (+), ICAM1 (+), IGF1R (+), I13R1 (+), |
| | TNR16 (+), PTPRC (+), SLAF1 (+), LEUK (+), TFR1 (+), TLR3 |
| | (+), TNFA (+), TNR8 (−), TNF11 (+), TNFL4 (+) |
| GO:0016021: integral | CD166 (+), AREG (+), BCAM (+), OX2G (−), PD1L1 (+), CD28 (+), |
| component of membrane | CD38 (+), CD4 (+), TNR5 (+), CD47 (+), CD81 (+), CD8A (+), |
| | CEAM1 (+), CSF1 (+), CXCR5 (+), DPP4 (−), ERBB2 (+), TNFL6 |
| | (+), GLPB (+), HAVR2 (−), ICAM1 (+), IGF1R (+), I13R1 (+), I13R2 |
| | (+), TNR16 (+), PTPRC (+), SLAF1 (+), LEUK (+), TFR1 (+), |
| | TLR3 (+), TNFA (+), TNR8 (−), TNF11 (+), TNFL4 (+) |
| GO:0044459: plasma | CD166 (+), BCAM (+), OX2G (−), PD1L1 (+), CD28 (+), CD38 (+), |
| membrane part | CD4 (+), TNR5 (+), CD47 (+), CD81 (+), CD8A (+), CEAM1 (+), |
| | CXCR5 (+), DPP4 (−), ERBB2 (+), TNFL6 (+), GLPB (+), HAVR2 |
| | (−), HMGB1 (+), ICAM1 (+), IGF1R (+), I13R1 (+), TNR16 (+), |
| | PTPRC (+), LEUK (+), TFR1 (+), TLR3 (+), TNFA (+), TNR8 (−), |
| | TNF11 (+), TNFL4 (+) |
| GO:0005615: extracellular | ADIPO (−), ALBU (−), AREG (+), CCL19 (−), CCL2 (+), CCL27 (+), |
| space | CCL8 (−), PD1L1 (+), CD47 (+), CD81 (+), CSF1 (+), TNFL6 (+), |
| | FGF2 (+), HMGB1 (+), ICAM1 (+), IL12B (+), I13R2 (+), IL15 (+), |
| | IL1A (+), IL2 (+), IL20 (+), IL3 (+), IL31 (−), LEUK (+), TGFB2 (+), |
| | TNFA (+), TNF11 (+), TNFL4 (+), VEGFA (+) |
| GO:0009986: cell surface | ADIPO (−), CD166 (+), AREG (+), BCAM (+), PD1L1 (+), CD28 |
| | (+), CD38 (+), CD4 (+), TNR5 (+), CD8A (+), CEAM1 (+), CXCR5 |
| | (+), DPP4 (−), TNFL6 (+), HAVR2 (−), HMGB1 (+), ICAM1 (+), |
| | IL12B (+), IL15 (+), TNR16 (+), PTPRC (+), SLAF1 (+), LEUK (+), |
| | TFR1 (+), TNFA (+), TNFL4 (+), VEGFA (+) |
| GO:0005887: integral | CD166 (+), BCAM (+), OX2G (−), CD28 (+), CD4 (+), TNR5 (+), |
| component of plasma | CD47 (+), CD81 (+), CD8A (+), CEAM1 (+), CXCR5 (+), TNFL6 |
| membrane | (+), GLPB (+), ICAM1 (+), IGF1R (+), I13R1 (+), TNR16 (+), |
| | PTPRC (+), LEUK (+), TFR1 (+), TLR3 (+), TNFA (+), TNR8 (−), |
| | TNF11 (+), TNFL4 (+) |
| GO:0031982: vesicle | ALBU (−), AREG (+), PD1L1 (+), CD38 (+), CD4 (+), CD47 (+), |
| | CD81 (+), CEAM1 (+), DPP4 (−), ERBB2 (+), TNFL6 (+), HAVR2 |
| | (−), HMGB1 (+), ICAM1 (+), IL12B (+), IL15 (+), TNR16 (+), |
| | PTPRC (+), TFR1 (+), TGFB2 (+), TLR3 (+), TNFA (+), VEGFA |
| | (+) |
| GO:0031410: cytoplasmic | ALBU (−), AREG (+), PD1L1 (+), CD38 (+), CD4 (+), CD47 (+), |
| vesicle | CEAM1 (+), DPP4 (−), ERBB2 (+), TNFL6 (+), HAVR2 (−), HMGB1 |
| | (+), IL12B (+), IL15 (+), TNR16 (+), PTPRC (+), TFR1 (+), TGFB2 |
| | (+), TLR3 (+), TNFA (+), VEGFA (+) |
| GO:0009897: external side of | CD166 (+), BCAM (+), PD1L1 (+), CD28 (+), CD4 (+), TNR5 (+), |
| plasma membrane | CD8A (+), CXCR5 (+), TNFL6 (+), ICAM1 (+), PTPRC (+), TFR1 |
| | (+), TNFA (+) |
| GO:0070062: extracellular | ALBU (−), PD1L1 (+), CD47 (+), CD81 (+), TNFL6 (+), ICAM1 (+) |
| exosome | |
| GO:0001772: immunological | CD166 (+), CD28 (+), CD81 (+), HAVR2 (−), ICAM1 (+) |
| synapse | |

TABLE 8
Selected biomarkers related to specific pathways (based on Kyoto Encyclopedia
of Genes and Genomes—“KEGG”) with differential abundance in acute CS vs MM.

| | |
|---|---|
| hsa04060: Cytokine-cytokine | CCL19 (−), CCL2 (+), CCL27 (+), CCL8 (−), TNR5 (+), CSF1 (+), |
| receptor interaction | CXCR5 (+), TNFL6 (+), IL12B (+), I13R1 (+), IL15 (+), IL1A (+), |
| | IL2 (+), IL20 (+), IL3 (+), TNR16 (+), TGFB2 (+), TNFA (+), TNR8 |
| | (−), TNF11 (+), TNFL4 (+), VEGFA (+) |
| hsa04010: MAPK signaling | AREG (+), CSF1 (+), ERBB2 (+), TNFL6 (+), FGF2 (+), IGF1R |
| pathway | (+), IL1A (+), TNR16 (+), TGFB2 (+), TNFA (+), VEGFA (+) |
| hsa05200: Pathways in cancer | ERBB2 (+), TNFL6 (+), FGF2 (+), IGF1R (+), IL12B (+), I13R1 (+), |
| | IL15 (+), IL2 (+), IL3 (+), TGFB2 (+), VEGFA (+) |
| hsa04151: PI3K-Akt signaling | AREG (+), CSF1 (+), ERBB2 (+), TNFL6 (+), FGF2 (+), IGF1R |
| pathway | (+), IL2 (+), IL3 (+), TNR16 (+), VEGFA (+) |
| hsa04514: Cell adhesion | CD166 (+), PD1L1 (+), CD28 (+), CD4 (+), TNR5 (+), CD8A (+), |
| molecules (CAMs) | ICAM1 (+), PTPRC (+), LEUK (+) |
| hsa04640: Hematopoietic cell | CD38 (+), CD4 (+), CD8A (+), CSF1 (+), IL1A (+), IL3 (+), TFR1 |
| lineage | (+), TNFA (+) |
| hsa04630: Jak-STAT signaling | IL12B (+), I13R1 (+), I13R2 (+), IL15 (+), IL2 (+), IL20 (+), IL3 (+) |
| pathway | |
| hsa05164: Influenza A | CCL2 (+), TNFL6 (+), ICAM1 (+), IL12B (+), IL1A (+), TLR3 (+), |
| | TNFA (+) |
| hsa04940: Type I diabetes | CD28 (+), TNFL6 (+), IL12B (+), IL1A (+), IL2 (+), TNFA (+) |
| mellitus | |
| hsa04660: T cell receptor | CD28 (+), CD4 (+), CD8A (+), IL2 (+), PTPRC (+), TNFA (+) |
| signaling pathway | |
| hsa05162: Measles | CD28 (+), TNFL6 (+), IL12B (+), IL1A (+), IL2 (+), SLAF1 (+) |
| hsa05168: Herpes simplex | CCL2 (+), TNFL6 (+), IL12B (+), IL15 (+), TLR3 (+), TNFA (+) |
| infection | |
| hsa04014: Ras signaling | CSF1 (+), TNFL6 (+), FGF2 (+), IGF1R (+), TNR16 (+), VEGFA |
| pathway | (+) |
| hsa05166: HTLV-I infection | TNR5 (+), ICAM1 (+), IL15 (+), IL2 (+), TGFB2 (+), TNFA (+) |
| hsa05321: Inflammatory bowel | IL12B (+), IL1A (+), IL2 (+), TGFB2 (+), TNFA (+) |
| disease (IBD) | |
| hsa04064: NF-kappa B signaling | CCL19 (−), TNR5 (+), ICAM1 (+), TNFA (+), TNF11 (+) |
| pathway | |
| hsa04668: TNF signaling | CCL2 (+), CSF1 (+), ICAM1 (+), IL15 (+), TNFA (+) |
| pathway | |
| hsa04062: Chemokine | CCL19 (−), CCL2 (+), CCL27 (+), CCL8 (−), CXCR5 (+) |
| signaling pathway | |
| hsa04015: Rap1 signaling | CSF1 (+), FGF2 (+), IGF1R (+), TNR16 (+), VEGFA (+) |
| pathway | |
| hsa05143: African | TNFL6 (+), ICAM1 (+), IL12B (+), TNFA (+) |
| trypanosomiasis | |
| hsa05340: Primary | CD4 (+), TNR5 (+), CD8A (+), PTPRC (+) |
| immunodeficiency | |
| hsa01521: EGFR tyrosine kinase | ERBB2 (+), FGF2 (+), IGF1R (+), VEGFA (+) |
| inhibitor resistance | |
| hsa04066: HIF-1 signaling | ERBB2 (+), IGF1R (+), TFR1 (+), VEGFA (+) |
| pathway | |
| hsa04620: Toll-like receptor | TNR5 (+), IL12B (+), TLR3 (+), TNFA (+) |
| signaling pathway | |
| hsa05152: Tuberculosis | IL12B (+), IL1A (+), TGFB2 (+), TNFA (+) |
| hsa05165: Human | TNFL6 (+), TLR3 (+), TNFA (+), VEGFA (+) |
| papillomavirus infection | |
| hsa05310: Asthma | TNR5 (+), IL3 (+), TNFA (+) |
| hsa05416: Viral myocarditis | CD28 (+), TNR5 (+), ICAM1 (+) |
| hsa04612: Antigen processing | CD4 (+), CD8A (+), TNFA (+) |
| and presentation | |
| hsa05133: Pertussis | IL12B (+), IL1A (+), TNFA (+) |
| hsa04658: Th1 and Th2 cell | CD4 (+), |
| differentiation | IL12B (+), IL2 (+) |
| hsa04650: Natural killer cell | TNFL6 (+), ICAM1 (+), TNFA (+) |
| mediated cytotoxicity | |
| hsa04068: FoxO signaling | TNFL6 (+), IGF1R (+), TGFB2 (+) |
| pathway | |
| hsa05160: Hepatitis C | CD81 (+), TLR3 (+), TNFA (+) |
| hsa04210: Apoptosis | TNFL6 (+), IL3 (+), TNFA (+) |
| hsa04930: Type II diabetes | ADIPO (−), TNFA (+) |
| mellitus | |
| hsa04664: Fc epsilon RI | IL3 (+), TNFA (+) |
| signaling pathway | |
| hsa04920: Adipocytokine | ADIPO (−), TNFA (+) |
| signaling pathway | |
| hsa04012: ErbB signaling | AREG (+), ERBB2 (+) |
| pathway | |
| hsa04350: TGF-beta signaling | TGFB2 (+), TNFA (+) |
| pathway | |
| hsa04657: IL-17 signaling | CCL2 (+), TNFA (+) |
| pathway | |

TABLE 9
Selected biomarkers related to specific molecular functions
with differential abundance in acute CS vs MM.

| | |
|---|---|
| GO:0005102: signaling | ADIPO (−), CD166 (+), AREG (+), CCL19 (−), CCL2 (+), CCL27 |
| receptor binding | (+), CCL8 (−), OX2G (−), CD4 (+), CD81 (+), CD8A (+), CSF1 (+), |
| | DPP4 (−), ERBB2 (+), TNFL6 (+), FGF2 (+), HMGB1 (+), ICAM1 |
| | (+), IGF1R (+), IL12B (+), IL15 (+), IL1A (+), IL2 (+), IL20 (+), IL3 |
| | (+), IL31 (−), TGFB2 (+), TNFA (+), TNF11 (+), TNFL4 (+), VEGFA |
| | (+) |
| GO:0005125: cytokine activity | ADIPO (−), AREG (+), CCL19 (−), CCL2 (+), CCL27 (+), CCL8 (−), |
| | CSF1 (+), TNFL6 (+), FGF2 (+), HMGB1 (+), IL12B (+), IL15 (+), |
| | IL1A (+), IL2 (+), IL20 (+), IL3 (+), IL31 (−), TGFB2 (+), TNFA (+), |
| | TNF11 (+), TNFL4 (+), VEGFA (+) |
| GO:0038023: signaling | BCAM (+), CD28 (+), CD4 (+), TNR5 (+), CD47 (+), CD8A (+), |
| receptor activity | CXCR5 (+), ERBB2 (+), ICAM1 (+), IGF1R (+), IL12B (+), I13R1 |
| | (+), I13R2 (+), TNR16 (+), PTPRC (+), SLAF1 (+), LEUK (+), |
| | TLR3 (+), TNR8 (−) |
| GO:0005126: cytokine | CCL19 (−), CCL2 (+), CCL27 (+), CCL8 (−), CSF1 (+), TNFL6 (+), |
| receptor binding | IL12B (+), IL15 (+), IL1A (+), IL2 (+), IL20 (+), IL3 (+), TGFB2 (+), |
| | TNFA (+), TNF11 (+), TNFL4 (+), VEGFA (+) |
| GO:0004888: transmembrane | BCAM (+), CD4 (+), TNR5 (+), CXCR5 (+), ERBB2 (+), ICAM1 |
| signaling receptor activity | (+), IGF1R (+), IL12B (+), I13R1 (+), I13R2 (+), TNR16 (+), |
| | PTPRC (+), SLAF1 (+), LEUK (+), TLR3 (+), TNR8 (−) |
| GO:0070851: growth factor | AREG (+), FGF2 (+), IL12B (+), IL1A (+), IL2 (+), IL3 (+), VEGFA |
| receptor binding | (+) |
| GO:0008083: growth factor | AREG (+), CSF1 (+), FGF2 (+), IL2 (+), IL3 (+), TGFB2 (+), |
| activity | VEGFA (+) |
| GO:0016301: kinase activity | CCL2 (+), CCL8 (−), CD28 (+), ERBB2 (+), FGF2 (+), IGF1R (+), |
| | IL3 (+) |
| GO:0001618: virus receptor | CD4 (+), CD81 (+), DPP4 (−), ICAM1 (+), SLAF1 (+), TFR1 (+) |
| activity | |
| GO:0004672: protein kinase | CCL2 (+), CCL8 (−), ERBB2 (+), FGF2 (+), IGF1R (+), IL3 (+) |
| activity | |
| GO:0004896: cytokine | CD4 (+), CXCR5 (+), IL12B (+), I13R1 (+), I13R2 (+) |
| receptor activity | |
| GO:0001664: G protein-coupled | CCL19 (−), CCL2 (+), CCL27 (+), CCL8 (−), IL2 (+) |
| receptor binding | |
| GO:0005164: tumor necrosis | TNFL6 (+), TNFA (+), TNF11 (+), TNFL4 (+) |
| factor receptor binding | |
| GO:0048020: CCR chemokine | CCL19 (−), CCL2 (+), CCL27 (+), CCL8 (−) |
| receptor binding | |
| GO:0008009: chemokine | CCL19 (−), CCL2 (+), CCL27 (+), CCL8 (−) |
| activity | |
| GO:0005178: integrin binding | CD81 (+), FGF2 (+), HMGB1 (+), ICAM1 (+) |
| GO:0004713: protein tyrosine | ERBB2 (+), FGF2 (+), IGF1R (+), IL3 (+) |
| kinase activity | |
| GO:0005031: tumor necrosis | TNR5 (+), TNR16 (+), TNR8 (−) |
| factor-activated receptor | |
| activity | |
| GO:0046934: phosphatidyl- | CD28 (+), ERBB2 (+), FGF2 (+) |
| inositol-4,5-bisphosphate 3- | |
| kinase activity | |
| GO:0019838: growth factor | IGF1R (+), TNR16 (+), TNR8 (−) |
| binding | |
| GO:0005149: interleukin-1 | IL1A (+), IL3 (+) |
| receptor binding | |
| GO:0042287: MHC protein | CD4 (+), CD8A (+) |
| binding | |

TABLE 10
Selected biomarkers related to specific pathways (based on Reactome
database) with differential abundance in acute CS vs MM.

| | |
|---|---|
| HSA-168256: Immune System | CCL19 (−), CCL2 (+), OX2G (−), PD1L1 (+), CD28 (+), CD4 (+), |
| | CD47 (+), CD81 (+), CD8A (+), CEAM1 (+), CSF1 (+), TNFL6 (+), |
| | FGF2 (+), HAVR2 (−), HMGB1 (+), ICAM1 (+), IL12B (+), I13R1 |
| | (+), I13R2 (+), IL15 (+), IL1A (+), IL2 (+), IL20 (+), IL3 (+), IL31 (−), |
| | PTPRC (+), TLR3 (+), TNFA (+), TNR8 (−), TNF11 (+), TNFL4 (+), |
| | VEGFA (+) |
| HSA-1280215: Cytokine | CCL19 (−), CCL2 (+), CD4 (+), CSF1 (+), TNFL6 (+), FGF2 (+), |
| Signaling in Immune system | HAVR2 (−), HMGB1 (+), ICAM1 (+), IL12B (+), I13R1 (+), I13R2 |
| | (+), IL15 (+), IL1A (+), IL2 (+), IL20 (+), IL3 (+), IL31 (−), TNFA (+), |
| | TNR8 (−), TNF11 (+), TNFL4 (+), VEGFA (+) |
| HSA-449147: Signaling by | CCL19 (−), CCL2 (+), CD4 (+), CSF1 (+), TNFL6 (+), FGF2 (+), |
| Interleukins | HAVR2 (−), HMGB1 (+), ICAM1 (+), IL12B (+), I13R1 (+), I13R2 |
| | (+), IL15 (+), IL1A (+), IL2 (+), IL20 (+), IL3 (+), IL31 (−), TNFA (+), |
| | VEGFA (+) |
| HSA-6785807: Interleukin-4 | CCL2 (+), TNFL6 (+), FGF2 (+), ICAM1 (+), IL12B (+), I13R1 (+), |
| and Interleukin-13 signaling | I13R2 (+), IL1A (+), TNFA (+), VEGFA (+) |
| HSA-1280218: Adaptive | OX2G (−), PD1L1 (+), CD28 (+), CD4 (+), CD81 (+), CD8A (+), |
| Immune System | ICAM1 (+), PTPRC (+) |
| HSA-109582: Hemostasis | ALBU (−), CD47 (+), CEAM1 (+), GLPB (+), LEUK (+), TGFB2 (+), |
| | VEGFA (+) |
| HSA-6783783: Interleukin-10 | CCL19 (−), CCL2 (+), CSF1 (+), ICAM1 (+), IL12B (+), IL1A (+), |
| signaling | TNFA (+) |
| HSA-451927: Interleukin-2 | HAVR2 (−), IL15 (+), IL2 (+), IL3 (+) |
| family signaling | |
| HSA-5668541: TNFR2 non- | TNFA (+), TNR8 (−), TNF11 (+), TNFL4 (+) |
| canonical NF-KB pathway | |
| HSA-198933: Immunoregulatory | OX2G (−), CD81 (+), CD8A (+), ICAM1 (+) |
| interactions between a | |
| Lymphoid and a non-Lymphoid | |
| cell | |
| HSA-202733: Cell surface | CD47 (+), CEAM1 (+), GLPB (+), LEUK (+) |
| interactions at the vascular wall | |
| HSA-5669034: TNFs bind | TNR8 (−), TNF11 (+), TNFL4 (+) |
| their physiological receptors | |
| HSA-380108: Chemokine receptors | CCL19 (−), CCL27 (+), CXCR5 (+) |
| bind chemokines | |
| HSA-389948: PD-1 signaling | PD1L1 (+), CD4 (+) |

### Example 3: Cluster Analysis of the Biomarkers Vs. The Different Patient Samples

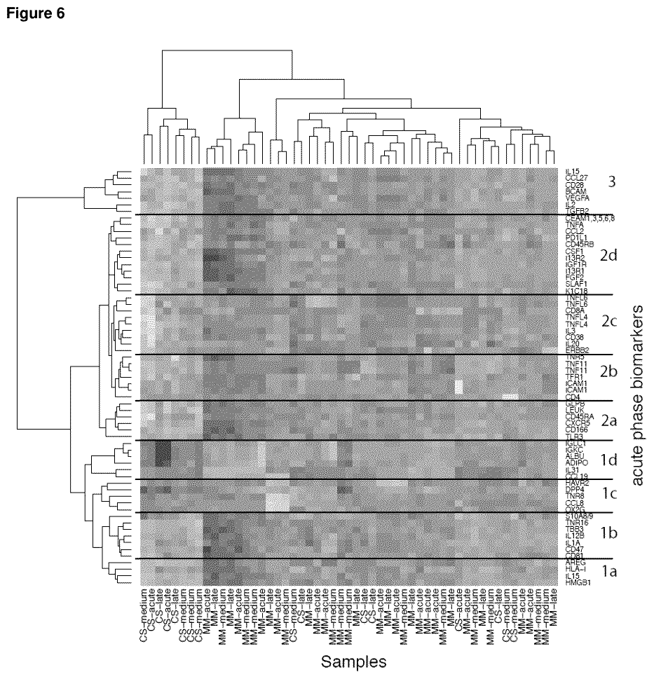

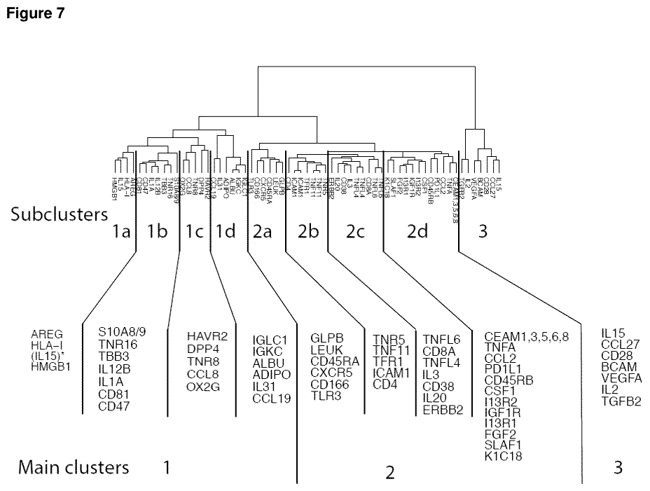

In order to group samples and biomarkers according the similarity of biomarker profiles, an unsupervised hierarchical cluster analysis was performed for the samples as well as for the biomarkers based on the M values (log 2 ratio of signal intensity in sample and reference sample) derived from the antibody array analysis. For the cluster analysis the euclidean distance and the complete linkage agglomeration method were used. The results are depicted in combination with biomarker-wise standardised M-values as a “heatmap” (see FIG. 6). White or light grey colours indicate a higher abundance in the respective samples, while black and darker grey colours indicate a lower abundance in the respective samples. Samples with a similar biomarker profile form clusters as indicated by the dendrogram at the top of the figure. At the left-hand side there is a distinct cluster of CS-patients. Also, the biomarkers from cluster of similar abundance profiles in the sample set. These clusters are indicated by the dendrogram at the left side of the plot. Respective clusters and their protein members are indicated at the right. In total three major biomarker groups (1, 2 and 3) could be identified, which are subdivided in sub-clusters 2a, 2b, 2c, 2d, 1a, 1b, 1c and 1d (see also FIG. 7 for more details).

Please note that in general biomarkers in clusters 1a, 1b, 2a, 2b, 2c, 2d, and 3 are significantly upregulated in most CS-patient samples, whereas biomarkers in clusters 1c and 1d are significantly downregulated in most CS-patients.

### Example 4: Additional Study in a Second Patient Cohort

In order to qualify the biomarkers further additional 106 plasma samples were selected to have matched critical/severe and mild/moderate patients on an antibody-microarray platform. All analysed samples were from the acute phase of infection, meaning less than 10 days after the onset of first symptoms.

TABLE 11
Patient characteristics 2nd cohort.

| | critical/severe | | mild/moderate |
|---|---|---|---|
| Number of samples | 53 | (47*) | 53 |
| Mean age | 62.05 | (60.2*) | 62.04 |
| male | 60.4% | (66%*) | 60.4% |
| female | 39.6% | (34%*) | 39.6% |
| days after onset of symptoms | 6.21 | (6.3*) | 5.87 |

*After filtering of six outlier patients (e.g. COVID-19 specific therapy)

During the data analysis process, 6 samples from patients with a critical or severe course of disease were filtered e.g. due to COVID-19 specific therapy before sample collection. Data analysis was performed as already described for the discovery cohort within the patent application.

All differentially abundant proteins from the first cohort (discovery cohort) were selected and checked whether they were differentially abundant in the 2nd cohort (validation cohort) as well. Some common targets were able to discriminate between a critical/severe and mild/moderate Covid-19 progression during an acute phase of infection within the two independent cohorts. These targets are shown in Table 12 together with their respective log FC and p-Values from both cohorts.

TABLE 12
Biomarkers identified in first and second cohort

| | | Discovery | | Validation | | |
|---|---|---|---|---|---|---|
| Biomarker/Short | SEQ | | adj. p- | | adj. p- | |
| protein name | ID No. | logFC | Val | logFC | Val | AUC |
| S10A8/A9 | 32/33 | 1.59 | 1.3E−02 | 1.58 | 4.83E−14 | 0.801 |
| CD81 | 44 | 1.54 | 3.8E−02 | 0.73 | 5.9E−03 | 0.722 |
| ALBU | 26 | −1.63 | 2.3E−03 | −0.55 | 1.5E−02 | 0.714 |
| AREG | 42 | 1.84 | 8.6E−03 | 1.30 | 1.4E−03 | 0.682 |
| FGF2 | 01 | 2.43 | 5.8E−06 | 1.19 | 2.1E−04 | 0.676 |
| CXCR5 | 15 | 2.94 | 3.7E−04 | 1.08 | 2.9E−03 | 0.669 |
| CD47 | 28 | 1.75 | 5.3E−04 | 1.10 | 1.3E−03 | 0.668 |
| SLAF1 | 27 | 2.41 | 3.7E−04 | 1.64 | 2.5E−05 | 0.666 |
| BTLA | 73 | 1.25 | 5.1E−02 | 1.12 | 1.8E−02 | 0.660 |
| TNR16 | 14 | 2.4 | 1.0E−04 | 0.98 | 1.0E−02 | 0.638 |
| I13R2 | 07 | 1.64 | 3.5E−06 | 0.89 | 3.4E−03 | 0.617 |
| IL2 | 21 | 2.72 | 3.4E−04 | 0.94 | 9.7E−03 | 0.614 |
| CD28 | 02 | 3.12 | 2.3E−05 | 0.70 | 8.4E−02 | 0.602 |

Additionally, the table shows the area under the curve (AUC) from the receiver operating characteristic (ROC) curve for the 2nd cohort (not available for 1st cohort due to limited sample size). Other biomarkers, identified within the discovery cohort did not exhibit significant differentiation in the 2nd cohort and are most likely describing differences caused by certain individuals or subgroups and might be suitable for identification of subgroups. Thus, one new method to rank these biomarkers as individual biomarkers is the area under the curve (AUC) from the receiver operating characteristic (ROC) curve for the 2nd cohort.

In addition, novel biomarkers were identified in the second cohort at a significance value of adjusted p.value <0.003 (table 13).

Biomarkers S10A8/A9, also known as calprotectin, as well as CRP, have been reported already as a potential diagnostic biomarker for COVID-19, showing the robustness of the present method.

TABLE 13
Additional biomarkers identified in second cohort.

| SEQ | | | Adj. |
|---|---|---|---|
| ID No. | Biomarker | logFC | p-Val |
| 106 | CRP | 2.79 | 1.10E−17 |
| 149 | FINC | −0.94 | 1.20E−05 |
| 107 | MUC1 | 1.65 | 6.70E−05 |
| 146 | TSP1 | −0.54 | 7.50E−05 |
| 112 | MPIP2 | 1.1 | 3.40E−04 |
| 111 | ACVL1 | 1.1 | 1.30E−03 |
| 108 | CALB1 | 1.23 | 1.30E−03 |
| 133 | MMP9 | 0.76 | 1.50E−03 |
| 118 | PRTN3 | 0.96 | 2.30E−03 |

### Example 5—Identification of Biomarker-Combinations with High Diagnostic Accuracy, Sensitivity and Sensibility

By using a machine-learning approach, biomarker-combinations were identified which can be used in diagnostics in order to improve accuracy, sensitivity and sensibility.

During this process, performance metrics were calculated for each biomarker.

In order to select further biomarkers outside of the cohort comparison (Table 12) and new biomarkers (Table 13), two metrics were considered in particular:
The average ROC AUC was calculated of all models containing a particular biomarker (Metric Ave.perf). If this average performance of a biomarker was high, it was assumed that said biomarker contributes positively to the classification models.
For each biomarker, the ratio of how many of the well-performing models (ROC AUC >0.8) contained that biomarker was calculated (Metric Freq.80). High ratios indicate that a biomarker was often part of well-performing classification models and thereby has special importance in classification models.
In addition, for each marker the AUC as individual marker is shown (Metric individual AUC), indicating that these markers improve the overall performance within classification models while they may not show a very clear differentiation signal as individual markers.

A number of new biomarkers were identified as playing a role in the well-performing classification models, and are listed in table 14.

TABLE 14
New biomarkers identified during the identification
of biomarker combinations

| | | Metric | Metric | Metric | | |
|---|---|---|---|---|---|---|
| SEQ | | Ave. | Freq. | individual | | adj. p- |
| ID No. | Biomarker | perf | 80 | AUC | logFC | Val |
| 115 | MTOR | 0.779 | 0.015 | 0.645 | 1.00 | 3.00E−03 |
| 150 | UTER | 0.772 | 0.027 | 0.536 | 0.27 | 8.90E−01 |
| 151 | CD14 | 0.771 | 0.033 | 0.679 | 0.44 | 9.90E−02 |
| 124 | CADH1 | 0.766 | 0.021 | 0.658 | 0.91 | 9.80E−03 |
| 142 | FABPI | 0.764 | 0.010 | 0.537 | 0.53 | 6.80E−01 |
| 129 | I22R2 | 0.764 | 0.016 | 0.701 | 0.85 | 9.70E−03 |
| 138 | IL26 | 0.763 | 0.028 | 0.637 | 0.65 | 7.70E−02 |
| 122 | SPIT1 | 0.761 | 0.009 | 0.690 | 0.93 | 7.00E−03 |
| 109 | ANGP2 | 0.760 | 0.016 | 0.663 | 1.22 | 6.30E−03 |
| 110 | CATB | 0.757 | 0.009 | 0.684 | 1.14 | 3.10E−02 |
| 113 | SFRP5 | 0.754 | 0.011 | 0.676 | 1.05 | 5.30E−03 |
| 139 | BASI | 0.752 | 0.012 | 0.577 | 0.63 | 5.00E−01 |
| 147 | S10AD | 0.751 | 0.014 | 0.617 | −0.65 | 4.40E−02 |
| 117 | SLIP | 0.750 | 0.012 | 0.677 | 0.98 | 5.40E−03 |

During the analysis ERBB2 turned out to be of interest, since although it did not majorly impact the overall classification performance of the model in terms of AUC/overall accuracy, it shifted the classification model towards higher sensitivity, which is desirable for clinical assays. Thus, ERBB2 may be combined with other biomarker-combinations in order to improve the overall sensitivity.

During the analysis in addition the biomarkers CCL2, HAVR2, TBB3, CD45RB and IGLC1 turned out to be of interest, since they impacted the classification performance of the model in terms of AUC/overall accuracy although not exhibiting a significant log FC in the validation cohort. Thus CCL2, HAVR2, TBB3, CD45RB and IGLC1 may be combined with other biomarker-combinations for increased accuracy.

### Example 6: Identification of Biomarker Combinations

For the screening of biomarker combinations, a combined list of biomarkers (1) identified as significant in cohort 1, (2) identified as significant in cohort 2 or (3) having shown promising performance in preliminary tests, was used.

Single decision tree classification models based on Gini impurity were trained using M-values of every possible combination of 3 biomarkers from this combined list as input data. A maximum leaf node number of 4 and minimum leaf size of 3 samples were selected as further parameters. Models were trained and tested on the whole validation study data set without splitting the data set into training and validation subsets.

ROC AUCs for each decision tree model were calculated using the predicted class probabilities of each sample after training, while sensitivity, specificity and F1 score were calculated from binary predictions of the model.

Promising biomarker combinations were identified via a combination of filtering by performance metrics, especially AUC and sensitivity, and manual screening considering factors such as antibody usage and sample split balance (table 15).

TABLE 15
shows the top performant biomarker combinations

| | Protein 1 | Protein 2 | Protein 3 | Protein 4 | | | |
|---|---|---|---|---|---|---|---|
| | (Seq | (Seq | (Seq | (Seq | | | |
| # | ID. No.) | ID. No.) | ID. No.) | ID. No.) | AUC | Sens | Spec |
| 1 | AREG (42) | CD81 (44) | FGF2 (01) | — | 0.758 | 0.83 | 0.58 |
| 2 | CD81 (44) | HAVR2 (54) | TBB3 (31) | — | 0.826 | 0.79 | 0.79 |
| 3 | CXCR5 (15) | CD81 (44) | FGF2 (01) | — | 0.806 | 0.85 | 0.66 |
| 4 | SLAF1 (27) | CXCR5 (15) | — | — | 0.758 | 0.77 | 0.68 |
| 5 | CXCR5 (15) | CD81 (44) | FGF2 (01) | — | 0.756 | 0.85 | 0.55 |
| 6 | CCL2 (57) | I13R2 (07) | — | — | 0.833 | 0.87 | 0.68 |
| 7 | CCL2 (57) | CD81 (44) | — | — | 0.816 | 0.83 | 0.68 |
| 8 | CCL2 (57) | FGF2 (01) | — | — | 0.815 | 0.87 | 0.64 |
| 9 | CCL2 (57) | CF47 (28) | — | — | 0.826 | 0.86 | 0.68 |
| 10 | UTER (150) | I13R2 (07) | — | — | 0.830 | 0.85 | 0.72 |
| 11 | S10A8/9 | CCL2 (57) | AREG (42) | ERBB2 (50) | n.a. | 0.91 | 0.79 |
| | (32/33) | | | | | | |
| 12 | S10A8/9 | CCL2 (57) | AREG (42) | — | n.a. | 0.81 | 0.89 |
| | (32/33) | | | | | | |
| 13 | S10A8/9 | CEAM1, 3, 5, | ERBB2 (50) | — | n.a. | 0.87 | 0.79 |
| | (32/33) | 6, 8 (37-41) | | | | | |
| 14 | S10A8/9 | CD45RB (13) | — | — | 0.882 | 0.87 | 0.81 |
| | (32/33) | | | | | | |
| 15 | S10A8/9 | CEAM1, 3, 5, | — | — | n.a. | 0.87 | 0.81 |
| | (32/33) | 6, 8 (37-41) | | | | | |
| 16 | S10A8/9 9 | CCL2 (57) | IL15 (05) | — | 0.882 | 0.87 | 0.83 |
| | (32/33) | | | | | | |
| 17 | S10A8/9 9 | CCL2 (57) | VEGF165b | — | 0.877 | 0.87 | 0.81 |
| | (32/33) | | (45, | | | | |
| 18 | S10A8/9 9 | ALBU (26) | CCL2 (57) | — | 0.940 | 0.87 | 0.91 |
| | (32/33) | | | | | | |
| 19 | S10A8/9 9 | ALBU (26) | I13R2 (07) | — | 0.937 | 0.81 | 0.96 |
| | (32/33) | | | | | | |
| 20 | S10A8/9 9 | ALBU (26) | IGF1R (09) | — | 0.932 | 0.83 | 0.93 |
| | (32/33) | | | | | | |
| 21 | S10A8/9 9 | CD8A (52) | CCL2 (57) | — | 0.928 | 0.83 | 0.93 |
| | (32/33) | | | | | | |
| 22 | S10A8/9 9 | ERBB2 (50) | — | — | 0.907 | 0.89 | 0.85 |
| | (32/33) | | | | | | |
| 23 | TSP1 (146) | AREG (42) | SLAF1 (27) | — | 0.851 | 0.74 | 0.89 |
| 24 | CCL2 (57) | IGLC1 (55) | I13R2 (07) | — | 0.847 | 0.89 | 0.68 |
| 25 | CD81 (44) | SLAF1 (27) | CD14 (151) | — | 0.832 | 0.87 | 0.68 |
| 26 | CD81 (44) | SLAF1 (27) | TSP1 (146) | — | 0.829 | 0.91 | 0.64 |
| 27 | CD81 (44) | TSP1 (146) | SLAF1 (27) | — | 0.827 | 0.72 | 0.85 |
| 28 | CD81 (44) | CD14 (151) | — | — | 0.823 | 0.81 | 0.68 |
| 29 | CD81 (44) | CD14 (151) | CXCR5 (15) | — | 0.821 | 0.81 | 0.75 |

### Example 7: Adding the Information from all Cohorts and Machine Learning

After considering all data from both cohorts and the combinatorial results from machine learning, the following biomarkers were confirmed to be of preferred importance when predicting and/or diagnosing a SARS-CoV-2 infection during acute state, all having a |log FC| of at least 0.5 (table 16).

TABLE 16
Biomarkers of specific importance when predicting and/or
diagnosing a SARS-CoV-2 infection during acute state

| | | Discovery | | Validation | | | Metric | Metric |
|---|---|---|---|---|---|---|---|---|
| SEQ | | | adj. P- | | adj. P- | | Ave. | Freq. |
| ID No. | Biomarker | logFC | Val. | logFC | Val. | AUC | perf | 80 |
| 27 | SLAF1 | 2.41 | 3.7E−04 | 1.64 | 2.5E−05 | 0.666 | 0.752 | 0.011 |
| 32/33 | S10A8/9 | 1.59 | 13E−02 | 1.58 | 4.8E−14 | 0.827 | 0.876 | 0.199 |
| 73 | BTLA | 1.27 | 1.6E−03 | 1.46 | 3.5E−03 | 0.66 | 0.742 | 0.010 |
| 42 | AREG | 1.84 | 8.6E−03 | 1.30 | 1.4E−03 | 0.682 | 0.752 | 0.009 |
| 01 | FGF2 | 2.43 | 5.8E−06 | 1.19 | 2.1E−04 | 0.676 | 0.757 | 0.012 |
| 28 | CD47 | 1.75 | 5.3E−04 | 1.10 | 1.3E−03 | 0.668 | 0.763 | 0.019 |
| 15 | CXCR5 | 2.94 | 3.7E−04 | 1.08 | 2.9E−03 | 0.669 | 0.749 | 0.010 |
| 14 | TNR16 | 2.4 | 1.0E−04 | 0.98 | 1.0E−02 | 0.638 | 0.752 | 0.011 |
| 07 | I13R2 | 1.64 | 3.5E−06 | 0.89 | 3.4E−03 | 0.617 | 0.757 | 0.014 |
| 09 | IGF1R | 2.69 | 5.9E−05 | 0.75 | 3.1E−01 | 0.663 | 0.748 | 0.009 |
| 44 | CD81 | 1.54 | 3.8E−02 | 0.73 | 5.9E−03 | 0.722 | 0.794 | 0.026 |
| 02 | CD28 | 3.12 | 2.3E−05 | 0.70 | 8.4E−02 | 0.602 | 0.727 | 0.008 |
| 45* | VEGF165b/ | 1.53 | 1.4E−02 | 0.68 | 6.8E−01 | 0.607 | 0.738 | 0.012 |
| | VEGFA | | | | | | | |
| 21 | IL2 | 2.72 | 3.4E−04 | 0.68 | 2.0E−01 | 0.607 | 0.721 | 0.007 |
| 05 | IL15 | 1.93 | 2.2E−02 | 0.63 | 7.3E−01 | 0.615 | 0.714 | 0.008 |
| 25 | HMGB1 | 1.62 | 1.9E−03 | 0.56 | 6.8E−01 | 0.607 | 0.719 | 0.008 |
| 26 | ALBU | 1.63 | 2.3E−03 | −0.55 | 1.5E−02 | 0.714 | 0.803 | 0.104 |

*VEGF165b is a splice variant of VEGFA

Additionally, the following new biomarkers were confirmed to be of preferred importance when predicting and/or diagnosing a SARS-CoV-2 infection during acute state, all having a |log FC| of at least 0.5 (table 17):

TABLE 17
New biomarkers of importance when predicting and/or
diagnosing a SARS-CoV-2 infection during acute state

| | Uniprot | | | | | | |
|---|---|---|---|---|---|---|---|
| | entry name | | | | Metric | Metric | Metric |
| SEQ | (short name | Uniprot | | Adj. P- | individual | Ave. | Freq. |
| ID No. | in brackets) | accession | logFC | Val | AUC | perf | 80 |
| 106 | CRP_HUMAN | P02741 | 2.79 | 1.1E−17 | 0.842 | 0.855 | 0.199 |
| 107 | MUC1_HUMAN | P15941 | 1.65 | 6.7E−05 | 0.654 | 0.763 | 0.020 |
| 108 | CALB1_HUMAN | P05937 | 1.23 | 1.3E−03 | 0.636 | 0.740 | 0.007 |
| 109 | ANGP2_HUMAN | O15123 | 1.22 | 6.3E−03 | 0.663 | 0.760 | 0.016 |
| 110 | CATB_HUMAN | P07858 | 1.14 | 3.1E−02 | 0.684 | 0.757 | 0.009 |
| 111 | ACVL1_HUMAN | P37023 | 1.10 | 1.3E−03 | 0.668 | 0.759 | 0.015 |
| 112 | MPIP2_HUMAN | P30305 | 1.10 | 3.4E−04 | 0.67 | 0.757 | 0.017 |
| 113 | SFRP5_HUMAN | Q5T4F7 | 1.05 | 5.3E−03 | 0.676 | 0.754 | 0.011 |
| 114 | IGF1_HUMAN | P05019 | 1.02 | 1.4E−02 | 0.644 | 0.736 | 0.009 |
| 115 | MTOR_HUMAN | P42345 | 1.00 | 3.0E−03 | 0.645 | 0.779 | 0.015 |
| 116 | FAF1_HUMAN | Q9UNN5 | 1.00 | 1.9E−01 | 0.603 | 0.740 | 0.008 |
| 117 | SLIP_HUMAN | Q68CJ6 | 0.98 | 5.4E−03 | 0.677 | 0.750 | 0.012 |
| 118 | PRTN3_HUMAN | P24158 | 0.96 | 2.3E−03 | 0.721 | 0.763 | 0.018 |
| 119 | TYRO3_HUMAN | Q06418 | 0.96 | 2.6E−02 | 0.64 | 0.747 | 0.010 |
| 120 | CADH5_HUMAN | P33151 | 0.96 | 2.3E−02 | 0.638 | 0.730 | 0.008 |
| 121 | S10AC_HUMAN | P80511 | 0.95 | 7.8E−03 | 0.673 | 0.747 | 0.014 |
| 122 | SPIT1_HUMAN | O43278 | 0.93 | 7.0E−03 | 0.69 | 0.761 | 0.009 |
| 123 | S100B_HUMAN | P04271 | 0.92 | 9.7E−03 | 0.661 | 0.739 | 0.009 |
| 124 | CADH1_HUMAN | P12830 | 0.91 | 9.8E−03 | 0.658 | 0.766 | 0.021 |
| 125 | LEG4_HUMAN | P56470 | 0.90 | 3.8E−02 | 0.661 | 0.741 | 0.009 |
| 126 | DMB_HUMAN | P28068 | 0.89 | 2.5E−02 | 0.666 | 0.745 | 0.009 |
| 127 | RARR2_HUMAN | Q99969 | 0.88 | 3.7E−03 | 0.61 | 0.745 | 0.010 |
| 128 | 2A5D_HUMAN | Q14738 | 0.86 | 1.7E−02 | 0.625 | 0.724 | 0.008 |
| 129 | I22R2_HUMAN | Q969J5 | 0.85 | 9.7E−03 | 0.701 | 0.764 | 0.016 |
| 130 | FGF9_HUMAN | P31371 | 0.85 | 1.4E−01 | 0.587 | 0.720 | 0.007 |
| 131 | CDN1A_HUMAN | P38936 | 0.82 | 4.3E−02 | 0.566 | 0.747 | 0.015 |
| 132 | ISK1_HUMAN | P00995 | 0.79 | 4.9E−02 | 0.642 | 0.726 | 0.008 |
| 133 | MMP9_HUMAN | P14780 | 0.76 | 1.5E−03 | 0.68 | 0.766 | 0.018 |
| 134 | SPRC_HUMAN | P09486 | 0.75 | 5.1E−01 | 0.608 | 0.712 | 0.007 |
| 135 | PEPC_HUMAN | P20142 | 0.71 | 6.0E−02 | 0.63 | 0.723 | 0.008 |
| 136 | ANGL3_HUMAN | Q9Y5C1 | 0.70 | 7.0E−02 | 0.659 | 0.746 | 0.011 |
| 137 | IBP1_HUMAN | P08833 | 0.67 | 3.4E−03 | 0.593 | 0.733 | 0.007 |
| 138 | IL26_HUMAN | Q9NPH9 | 0.65 | 7.7E−02 | 0.637 | 0.763 | 0.028 |
| 139 | BASI_HUMAN | P35613 | 0.63 | 5.0E−01 | 0.577 | 0.752 | 0.012 |
| 140 | CORIN_HUMAN | Q9Y5Q5 | 0.63 | 4.8E−01 | 0.581 | 0.728 | 0.007 |
| 141 | IFI27_HUMAN | P40305 | 0.55 | 2.7E−01 | 0.625 | 0.723 | 0.006 |
| 142 | FABPI_HUMAN | P12104 | 0.53 | 6.8E−01 | 0.537 | 0.764 | 0.010 |
| 143 | IBP2_HUMAN | P18065 | 0.50 | 1.2E−01 | 0.631 | 0.726 | 0.009 |
| 144 | AMBP_HUMAN | P02760 | −0.50 | 1.4E−01 | 0.631 | 0.740 | 0.008 |
| 145 | IL1B_HUMAN | P01584 | −0.52 | 8.2E−01 | 0.501 | 0.718 | 0.007 |
| 146 | TSP1_HUMAN | P07996 | −0.54 | 7.5E−05 | 0.719 | 0.808 | 0.119 |
| 147 | S10AD_HUMAN | Q99584 | −0.65 | 4.4E−02 | 0.617 | 0.751 | 0.014 |
| 148 | TOP1_HUMAN | P11387 | −0.78 | 5.0E−02 | 0.582 | 0.727 | 0.007 |
| 149 | FINC_HUMAN | P02751 | −0.94 | 1.2E−05 | 0.733 | 0.779 | 0.030 |
| 150 | UTER_HUMAN* | P11684 | 0.27 | 8.9E−01 | 0.536 | 0.772 | 0.130 |
| 151 | CD14_HUMAN* | P08571 | 0.44 | 9.9E−02 | 0.679 | 0.771 | 0.170 |

*targets were important in machine learning approach but showed logFC values <0.5

1-17. (canceled)

18. A method for determining and treating a predisposition for a severe or critical course of a COVID-19-disease from a mild or moderate course of a COVID-19-disease in a subject having COVID-19, comprising identifying the subject as having the predisposition for the severe or critical course of a COVID-19 in an assay, comprising:
determining in a sample obtained from the subject the amount of a combination of at least two biomarkers selected from the group consisting of TSP1 and S10A8/9, as well as isoforms, fragments and/or variants thereof; and
determining the difference of the amount of said at least two biomarkers to a reference amount for said at least two biomarkers;
wherein the sample is obtained at a specific time-phase, acute, median or late, of the COVID-19 disease;
and wherein the reference amount is the amount of the respective biomarker in a subject who has a mild or moderate COVID-19-disease; and
treating the identified subject by:
administering passive ventilation when blood oxygen level is greater than 93%; or
administering a therapeutic agent to the subject, wherein the therapeutic agent is selected from the group consisting of an antiviral drug, a serine protease, an antibiotic, a cytokine inhibitor, an anti-parisitic agent, an anticoagulant, a glucocorticoid, lndinavir, bamlanivimab (LY-CoV555), convalescent plasma, ltolizumab, etesevimab (JS016/LYCoV016), casirivimab+imdevimab (REGN-COV2), Sotrovimab (VIR-7831), favipiravir, Regkirona, camostat, Plitidepsin, AT-527 (Altea Pharmaceuticals), AZD7442 (AstraZeneca), AZD1061 (AstraZeneca), AZD8895 (AstraZeneca), MP0420 (molecular partners), ATR-002 (Atriva Therapeutics), XVR011 (ExeVir Bio), COR-101 (Corat Therapeutics), Vilobelimab (lnflarx), IFX-2 (lnflarx), ISA106 (ISA Pharmaceuticals), Aviptadil, Remdesivir (GS-5734), anakinra, Olumatlizumab, baricitinib, apremilast, mCBM40, valsartan, omeprazole, nintedanib, methylprednisolone, linagliptin (Tradjenta), lenalidomide (Revlimid), hyrocortisone, cyclosporine, atorvastatin, artemisin, tavalisse, symbicort, RecAP, pulmicort and prednisone.

19. The method according to claim 18, wherein the difference of the amount of said each of the at least two biomarkers of Ilog FCl is at least 0.5, as compared to the reference amount indicates that the subject has a predisposition for a severe or critical course of a COVID-19-disease.

20. The method according to claim 18, wherein said difference is determined to be statistically significant by having an adjusted p-value of less than 5*10⁻².

21. The method according to claim 18, wherein the combination of at least two biomarkers in the sample from the subject comprises at least one further biomarker.

22. The method according to claim 22, wherein the at least one further biomarker is selected from the group consisting of SLAF1, BTLA, AREG, FGF2, CD47, CXCR5, TNR16, I13R2, IGF1R, CD81, CD28, VEGF165b/VEGFA, IL2, IL15, HMGB1, ALBU, MUC1, CCL3, CALB1, ANGP2, CATB, ACVL1, MPIP2, SFRP5, IGF1, MTOR, FAF1, SLIP, PRTN3, TYRO3, CADH5, S10AC, SPIT1, S100B, CADH1, LEG4, DMB, RARR2, 2A5D, I22R2, FGF9, CDN1A, ISK1, MMP9, SPRC, PEPC, ANGL3, IBP1, IL26, BASI, CORIN, IFI27, FABPI, IBP2, AMBP, IL1B, S10AD, CEAM1,3,5,6,8, ERBB2, FINC and TOP1, as well as combinations thereof and/or isoforms, fragments and/or variants thereof.

23. The method according to claim 22, wherein in the at least one further biomarker comprises a combination of biomarkers selected from the group consisting of: AREG, CD81 and FGF2; CD81, HAVR2 and TBB3; CXCR5, CD81 and FGF2; SLAF1 and CXCR5; CCL2 and I13R2; CCL2 and CD81; CCL2 and FGF2; CCL2 and CF47; UTER and I13R2; CCL2, AREG and ERBB2; CCL2 and AREG; CEAM1,3,5,6,8 and ERBB2; and CD45RB; CCL2 and IL15; CCL2 and VEGF165b; ALBU and CCL2; ALBU and I13R2; ALBU and IGF1R; CD8A and CCL2; AREG and SLAF1; CCL2, IGLC1 and I13R2; CD81, SLAF1 and CD14; CD81 and SLAF1; CD81 and CD14; CD81, CD14 and CXCR5, as well as combinations thereof and/or isoforms, fragments and/or variants thereof.

24. The method according to claim 18, wherein the at least one further biomarker comprises at least two biomarkers selected from the group consisting of ALBU, AREG, BTLA, CD81, CD28, CD47, CXCR5, FGF2, HMGB1, I13R2, IGF1R, IL2, IL15, SLAF1, TNR16, VEGF165b/VEGFA, HAVR2, TBB3; CCL2, CCL2, CF47; UTER, ERBB2, CEAM1,3,5,6,8, CD45RB, IGF1R, CD8A, IGLC1, and CD14, as well as combinations thereof and/or isoforms, fragments and/or variants thereof.

25. The method according to claim 18, wherein the at least one further biomarker is at least two biomarkers selected from at least two different cluster-groups selected from cluster 1-group, cluster 2-group and cluster 3-group.

26. The method according to claim 18, wherein the time-phase when the sample is taken is less than 9 days after the subject has been tested positive for a SARS-CoV-2-infection and/or from the onset of first symptoms.

27. The method according to claim 18, further comprising determining in the sample the amount is upregulated for at least one further biomarker selected from the group consisting of TNR5, TNF11, CD38, CD4, IL1A, TFR1, TNFL6, CD8A, IL20, CCL2, IL12B, VEGFA, HLA-I, ICAM1, CD81, ERBB2, HMGB1, I13R2, CCL27, K1C18, CD47, TBB3, AREG, CD45RA, TNFL4, IL3, CD45RB, CEAM1/3/5/6/8, GLPB, I13R1, PD1L1, LEUK, BCAM, TNR16, SLAF1, FGF2, CD166, TNFA, IGF1R, IL2, CSF1, TLR3, IL15, TGFB2, CXCR5, and CD28, as well as combinations thereof and/or isoforms, fragments and/or variants thereof.

28. The method according to claim 18, including determining in the sample the amount is downregulated for at least one further biomarker selected from the group consisting of TNR8, OX2G, CCL19, IGKC, ALBU, CCL8, DPP4, HAVR2, IGLC1, IL31, and ADIPO as well as combinations thereof and/or isoforms, fragments and/or variants thereof.

29. The method according to claim 18, wherein the at least one further biomarker is selected from the group consisting of ERBB2, interferon lambda and FINC; as well as combinations thereof and/or isoforms, fragments and/or variants thereof.

30. The method according to claim 18, wherein an effectiveness of a medical intervention and/or treatment is determined based on the biomarker profile of said patient in a sample selected from the group consisting of urine, blood, plasma and serum.

31. A method for determining significant upregulation or downregulation of a combination of biomarkers in a subject having COVID-19, comprising:
a. determining in a sample obtained from the subject the amount of each of the biomarkers in the combination of biomarkers by exposing the sample to immobilized antibodies specific for said each of the biomarkers and quantifying the amount of the biomarkers bound to the immobilized antibodies; and
b. determining the difference of the amount of said each of the at least two biomarkers to a reference amount for said each of the at least two biomarkers;
wherein the sample is obtained at a specific time-phase, acute, median or late, of the COVID-19 disease;
wherein the reference amount is the amount of the respective biomarker in a subject who has a mild or moderate COVID-19-disease at the same specific time-phase; and
wherein the combination of the at least two biomarkers is selected from the group consisting of TSP1 and S10A8/9, as well as isoforms, fragments and/or variants thereof.

32. The method according to claim 31, wherein the sample is taken prior to a planned COVID-19 medical intervention and/or therapy.

33. The method according to claim 32, further comprising carrying out the planned medical intervention and/or therapy, wherein the planned medical intervention and/or therapy is selected from the group consisting of administration of a drug, avoiding administration of a drug, artificial respiration treatment, extracorporeal membrane oxygenation (ECMO) and a surgical intervention.

34. The method according to claim 31, wherein the difference of the amount of said each of the at least two biomarkers of |log FCl is at least 0.5, as compared to the reference amount indicates that the subject has a predisposition for a severe or critical course of a COVID19-disease.

35. The method according to claim 31, wherein said difference is determined to be statistically significant by having an adjusted p-value of less than 5*10⁻².

36. The method according to claim 31, wherein the combination of at least two biomarkers in the sample from the subject comprises at least one further biomarker.

37. The method according to claim 36, wherein the at least one further biomarker is selected from the group consisting of SLAF1, BTLA, AREG, FGF2, CD47, CXCR5, TNR16, I13R2, IGF1R, CD81, CD28, VEGF165b/VEGFA, IL2, IL15, HMGB1, ALBU, MUC1, CCL3, CALB1, ANGP2, CATB, ACVL1, MPIP2, SFRP5, IGF1, MTOR, FAF1, SLIP, PRTN3, TYRO3, CADH5, S10AC, SPIT1, S100B, CADH1, LEG4, DMB, RARR2, 2A5D, I22R2, FGF9, CDN1A, ISK1, MMP9, SPRC, PEPC, ANGL3, IBP1, IL26, BASI, CORIN, IFI27, FABPI, IBP2, AMBP, IL1B, S10AD, CEAM1,3,5,6,8, ERBB2, FINC and TOP1, as well as combinations thereof and/or isoforms, fragments and/or variants thereof.

38. The method according to claim 36, wherein in the at least one further biomarker comprises a combination of biomarkers selected from the group consisting of: AREG, CD81 and FGF2; CD81, HAVR2 and TBB3; CXCR5, CD81 and FGF2; SLAF1 and CXCR5; CCL2 and I13R2; CCL2 and CD81; CCL2 and FGF2; CCL2 and CF47; UTER and I13R2; CCL2, AREG and ERBB2; CCL2 and AREG; CEAM1,3,5,6,8 and ERBB2; and CD45RB; CCL2 and IL15; CCL2 and VEGF165b; ALBU and CCL2; ALBU and I13R2; ALBU and IGF1R; CD8A and CCL2; AREG and SLAF1; CCL2, IGLC1 and I13R2; CD81, SLAF1 and CD14; CD81 and SLAF1; CD81 and CD14; CD81, CD14 and CXCR5, as well as combinations thereof and/or isoforms, fragments and/or variants thereof.

39. The method according to claim 18, wherein the at least one further biomarker comprises at least two biomarkers selected from the group consisting of ALBU, AREG, BTLA, CD81, CD28, CD47, CXCR5, FGF2, HMGB1, I13R2, IGF1R, IL2, IL15, SLAF1, TNR16, VEGF165b/VEGFA, HAVR2, TBB3; CCL2, CCL2, CF47; UTER, ERBB2, CEAM1,3,5,6,8, CD45RB, IGF1R, CD8A, IGLC1, and CD14, as well as combinations thereof and/or isoforms, fragments and/or variants thereof.

40. The method according to claim 18, wherein the at least one further biomarker is at least two biomarkers selected from at least two different cluster-groups selected from cluster 1-group, cluster 2-group and cluster 3-group.

41. The method according to claim 18, wherein the time-phase when the sample is taken is less than 9 days after the subject has been tested positive for a SARS-CoV-2-infection and/or from the onset of first symptoms.

42. The method according to claim 18, further comprising determining in the sample the amount is upregulated for at least one further biomarker selected from the group consisting of TNR5, TNF11, CD38, CD4, IL1A, TFR1, TNFL6, CD8A, IL20, CCL2, IL12B, VEGFA, HLA-I, ICAM1, CD81, ERBB2, HMGB1, I13R2, CCL27, K1C18, CD47, TBB3, AREG, CD45RA, TNFL4, IL3, CD45RB, CEAM1/3/5/6/8, GLPB, I13R1, PD1L1, LEUK, BCAM, TNR16, SLAF1, FGF2, CD166, TNFA, IGF1R, IL2, CSF1, TLR3, IL15, TGFB2, CXCR5, and CD28, as well as combinations thereof and/or isoforms, fragments and/or variants thereof.

43. The method according to claim 18, including determining in the sample the amount is downregulated for at least one further biomarker selected from the group consisting of TNR8, OX2G, CCL19, IGKC, ALBU, CCL8, DPP4, HAVR2, IGLC1, IL31, and ADIPO as well as combinations thereof and/or isoforms, fragments and/or variants thereof.

44. The method according to claim 18, wherein the at least one further biomarker is selected from the group consisting of ERBB2, interferon lambda and FINC; as well as combinations thereof and/or isoforms, fragments and/or variants thereof.
